SEQUENCE LISTING (11 sequences)

SEQ ID NO: 1 - protein, 18 aa, synthetic construct
  REGION 1..18: note = linker
KESGSVSSEQLAQFRSLD

SEQ ID NO: 2 - protein, 14 aa, synthetic construct
  REGION 1..14: note = linker
EGKSSGSGSESKST

SEQ ID NO: 3 - protein, 12 aa, synthetic construct
  REGION 1..12: note = linker
GSAGSAAGSGEF

SEQ ID NO: 4 - protein, 5 aa, synthetic construct
  REGION 1..5: note = linker
EAAAK

SEQ ID NO: 5 - protein, 7 aa, synthetic construct
  REGION 1..7: note = linker
AEAAAKA

SEQ ID NO: 6 - protein, 118 aa, synthetic construct
  REGION 1..118: note = anti-FcgammaRI heavy chain variable region
QFQLVQSGPELKKPGETVKISCKASGYTFTTYGMSWVKQAPGKGLKWMGWINTYSGMPTYGDDFKGRFAFSLETSASTAYLQINNLKNEDTATYFCARNWDDWYFDVWGTGTTVTVSS

SEQ ID NO: 7 - protein, 107 aa, synthetic construct
  REGION 1..107: note = anti-FcgammaRI light chain variable region
DIQMTQSPASLAASVGETITITCQASENIYFSLAWYQQKQGKSPQLLIYNANTLKHGVPSRFSGSGSGTQYSMKINNLQPEDTATYFCKQPYGFPRTFGGGTKLEIK

SEQ ID NO: 8 - protein, 121 aa, synthetic construct
  REGION 1..121: note = anti-FcgammaRIIb heavy chain variable region
EVQLQQSGGGLVQPGRSLKLSCAASGFTFSDYYMAWVRQAPTTGLEWVASISYDGGDTHYRDSVKGRFTISRDNAKSSLYLQMDSLRSEDTATYYCATETTGIPTGVMDAWGQGVSVTVSS

SEQ ID NO: 9 - protein, 107 aa, synthetic construct
  REGION 1..107: note = anti-FcgammaRIIb light chain variable region
DVQMTQSPYNLAASPGESVSINCKASESISKYLAWYLQKPGKANKLLMYDGSTLQSGIPSRFSGSGSGTDFTLTIRSLEPEDFGLYYCQQHYEYPATFGSGTKLEIK

SEQ ID NO: 10 - protein, 5 aa, synthetic construct
  source 1..5: note = synthetic GGGGS linker
GGGGS

SEQ ID NO: 11 - protein, 9 aa, synthetic construct
  source 1..9: note = synthetic influenza virus hemagglutinin
YPYDVPDYA

(19) United States
(12) Patent Application Publication
(10) Pub. No.: US 2025/0109216 A1
(43) Pub. Date: Apr. 3, 2025

(54) COMPOSITIONS AND METHODS FOR CROSSLINKING FC RECEPTORS

(71) Applicant: The University of North Carolina at Chapel Hill, Chapel Hill, NC (US)

(72) Inventor: Barbara Vilen, Chapel Hill, NC (US)

(21) Appl. No.: 18/981,002

(22) Filed: Dec. 13, 2024

Related U.S. Application Data
(62) Division of application No. 17/573,145, filed on Jan. 11, 2022, now abandoned.
Application No. 17/573,145 is a continuation of application No. 16/335,989, filed on Mar. 22, 2019, now abandoned.
(60) Provisional application No. 62/402,323, filed on Sep. 30, 2016.

(51) Int. Cl. C07K 16/46; C07K 16/28

(52) U.S. Cl. CPC C07K 16/468; C07K 16/283; C07K 2317/31; C07K 2317/55; C07K 2317/62; C07K 2317/64

(57) ABSTRACT

The present invention provides methods of treating an autoimmune disorder comprising administering to the subject an effective amount of a molecule comprising molecules having multispecificity, such as wherein at least one of a first immunoglobulin fragment antigen binding region (Fab1) and at least one of a second immunoglobulin fragment antigen binding region (Fab2), wherein Fab1 is specific for FcγRI or FcγRIIa, and wherein Fab2 is specific for FcγRIIb.

### PRIORITY STATEMENT

This application is a divisional application of U.S. patent application Ser. No. 17/573,145, filed Jan. 11, 2022, which is a continuation application of U.S. patent application Ser. No. 16/335,989, filed Mar. 22, 2019, which is a 35 U.S.C. § 371 national phase application of International Application Serial No. PCT/US2017/054557, filed Sep. 29, 2017, which claims the benefit, under 35 U.S.C. § 119(e), of U.S. Provisional Application Ser. No. 62/402,323, filed Sep. 30, 2016, the entire contents of each of which are incorporated by reference herein.

### STATEMENT OF GOVERNMENT SUPPORT

This invention was made with government support under Grant Numbers AI105613 and AI007098 awarded by the National Institutes of Health. The government has certain rights in the invention.

### STATEMENT REGARDING ELECTRONIC FILING OF A SEQUENCE LISTING

A Sequence Listing in XML format, entitled 5470-825CTDV_ST26.xml, 14,722 bytes in size, generated on Dec. 13, 2024, and filed herewith, is hereby incorporated by reference in its entirety for its disclosures.

### FIELD OF THE INVENTION

This invention is directed to compositions having multiple specificities for different targets and methods of their use to treat disorders such as systemic lupus erythematosus (SLE).

### BACKGROUND

Apoptotic debris, anti-nuclear autoantibodies, and their formation of immune complexes (IgG-ICs) have long been implicated in systemic lupus erythematosus (SLE); however, the molecular events that lead to the accumulation of apoptotic debris and/or IgG-ICs, and the events they induce that disrupt immune tolerance are not clear. A better understanding of these early events is needed so that therapeutics can be designed to target events upstream of multiple disease-related defects.

The present invention overcomes previous shortcomings in the art by providing compositions and methods for treatment of disorders such as SLE.

### SUMMARY OF THE INVENTION

This summary lists several embodiments of the presently disclosed subject matter and in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides a synthetic molecule comprising at least one of a first immunoglobulin fragment antigen binding region (Fab1) and at least one of a second fragment antigen binding region (Fab2), wherein Fab1 comprises a first heavy chain comprising a variable region (first V_{H}), and a first light chain comprising a variable region (first V_{L}), wherein the first V_{H} and the first V_{L} form a first antigen binding site specific for an activating Fcγ receptor, and wherein Fab2 comprises a second heavy chain comprising a variable region (second V_{H}), and a second light chain comprising a variable region (second V_{L}), and the second V_{H} and the second V_{L} form a second antigen binding site specific for an inhibitory Fcγ receptor. In some embodiments, the synthetic molecule can also comprise at least one of a third fragment antigen binding region (Fab3), wherein Fab3 comprises a third heavy chain comprising a variable region (third V_{H}) and a third light chain comprising a variable region (third V_{L}), wherein the third V_{H} and the third V_{L} form a third antigen binding site specific for an inhibitory Fcγ receptor. In some embodiments, the synthetic molecule can also comprise at least one of a fourth fragment antigen binding region (Fab4), wherein Fab4 comprises a fourth heavy chain comprising a variable region (fourth V_{H}) and a fourth light chain comprising a variable region (fourth V_{L}), wherein the fourth V_{H} and the fourth V_{L} form a fourth antigen binding site specific for an activating Fcγ receptor.

In some embodiments, Fab1 and Fab4 are specific for FcγRI or FcγRIIa and in some embodiments, Fab2 and Fab3 are specific for FcγRIIb

In additional embodiments of this invention, the synthetic molecule described above can further comprise at least one immunoglobulin constant region that comprises at least one heavy chain (C_{H}) and/or at least one light chain (C_{L}).

In further embodiments of this invention, the synthetic molecule described above can further comprise at least one immunoglobulin constant region that comprises at least one immunoglobulin Fc region.

In particular embodiments, the synthetic molecule of this invention can comprise any combination of Fab1, Fab2, Fab3, Fab4, C_{H}, C_{L} and/or Fc, linked to one another in any combination and in any multiples of Fab1, Fab2, Fab3, Fab4, C_{H}, C_{L} and/or Fc. The Fab1, Fab2, Fab3, Fab4, C_{H}, C_{L} and/or Fc moieties can be linked to one another via a linker molecule.

In some embodiments, the present invention provides a composition comprising a first synthetic molecule comprising at least one Fab1 specific for FcγRI and at least one Fab2 specific for FcγRIIb and a second synthetic molecule comprising at least one Fab1 specific for FcγRIIa and at least one Fab2 specific for FcγRIIb. In some embodiments of said composition, the first synthetic molecule comprises at least one Fab3 specific for FcγRIIb and/or the second synthetic molecule comprises at least one Fab3 specific for FcγRIIb. In some embodiments of said composition, the first synthetic molecule further comprises at least one Fab4 specific for FcγRI or FcγRIIa and/or the second synthetic molecule further comprises at least one Fab4 specific for FcγRI or FcγRIIa.

In additional embodiments of said composition, the first synthetic molecule and/or the second synthetic molecule comprises at least one immunoglobulin constant region that comprises at least one heavy chain (C_{H}) and/or at least one light chain (C_{L}), and/or wherein the first synthetic molecule and/or the second synthetic molecule comprises at least one immunoglobulin Fc region.

In additional embodiments of said composition, the first synthetic molecule and the second synthetic molecule are linked to any one or more of any Fab1, Fab2, Fab3, Fab4, C_{H}, C_{L} and/or Fc present, in any combination and in any multiplicity of first synthetic molecules and second synthetic molecules.

The present invention further provides methods of treatment of disorders such as an autoimmune disorder, systemic lupus erythematosus (SLE), diabetes and arthritis in a subject, comprising administering to the subject an effective amount of a synthetic molecule, composition, nucleic acid molecule and/or cell of this invention.

Other objects and advantages will become apparent upon a review of the following description and figures.

### BRIEF DESCRIPTION OF THE DRAWINGS

FIGS. 1A-1J. Nonlimiting examples of synthetic molecules of this invention, comprising Fab1, Fab2, Fab3, Fab4, C_{H}, C_{L} and/or Fc in various combinations and orientations and multiplicities. Of note regarding the embodiments of the present invention are the tandem Fab-IgG (Orthogonal Fab) shown in FIG. 1G, the appended IgGs-HC fusions shown in FIG. 1H, the DNL-Fab₄-IgG shown in FIG. 1C and the appended IgGs-HC & LC fusions shown in FIG. 1I.

FIG. 2. Model of lysosome defects in MRL/lpr mice. In lupus-prone mice, chronic activation of the PI3k pathway disrupts lysosomal acidification and induces undegraded IgG-ICs to recycle back to the cell surface. This induces a feedforward loop of chronic FcγRI signaling and diminished lysosomal acidification. Further, SHIP1 is not activated, and is mislocalized within the cells; however, these defects are corrected by crosslinking FcγRI with FcγRIIb.

FIGS. 3A-3B. The levels of tyrosine phosphorylation of FcγRI are elevated in MRL/lpr BMMφs. (3A-3B). BMMφs (8−10×10\hat{ }6 cells) from B6 and MRL/lpr were stimulated with IgG-ICs, and at indicated time points cells were lysed. Lysates were immunoprecipitated for FcγRI and beads were resolved using SDS-PAGE. Membranes were immunoblotted for pTyr (3A) and FcγRI (3B). n=3.

FIGS. 4A-4D. MRL/lpr Mφs have heightened PIP₃ and fail to produce PI(3, 2)P₂. (4A-4B). BMMφs from B6 and MRL/lpr were stimulated with IgG-ICs and PIP3 (4A) and PI(3, 4)P2 (4B) levels were assessed by flow cytometry; 4 exp, 4 mice. The MFI was normalized to the isotype control and relative to unstimulated B6. (4C-4D). Cell lysates from unstimulated BMMφs (1−1.5×10⁶ cells) derived from B6 and MRL/lpr mice were analyzed by immunoblot; 4 experiments, 4 mice. Densitometry values were normalized to tubulin and relative to B6.

FIGS. 5A-5B. Localization of mTOR is defective in MRL/lpr BMMφ. (5A-5B). Unstimulated BMMφs cultured±Torin1 (250 ng/mL) for 2 hrs were examined for mTOR localization with CD11b by confocal imaging. Colocalization of mTOR to membrane (CD11b) is quantitated by mander's coefficient (5A). Total cellular mTOR levels were quantitated using ImageJ software (5B); 3 exp, 2-3 mice, 10-29 cells.

FIGS. 6A-6B. Inhibiting PI3K or mTOR activity restores lysosome acidification and diminishes the levels of recycled IgG-ICs. (6A). BMMφs from B6 and MRL/lpr mice were stimulated with IgG-ICs±TGX-221 (p110β), IC87114 (p110δ), or Torin1 (mTorC1/C2), and ratiometric flow cytometry was used to quantify the relative phagosomal pH. (6B). Cells were pre-treated with drugs (for 2 hr) and given IgG-ICs. IgG-ICs were removed after 2 hr. Cells remained on drug for the duration of the experiment. At 72 hrs, levels of surface bound IgG-ICs were analyzed by flow cytometry. All values are normalized to B6 untreated. n=3-7.

FIGS. 7A-7D. MRL/lpr Mφs harbor defective phosphorylation of SHIP1 and fail to recruit pSHIP1 to the plasma membrane, lipid rafts, and FcγRI following the stimulation of IgG-ICs. (7A). BMMφs from B6 and MRL/lpr mice were stimulated with IgG-ICs for indicated time points and the levels of pSHIP1 or total SHIP1 were measured by flow cytometry. The levels of phospho-protein were normalized to total protein levels, and the fold changes over B6 mice at time 0 was graphed. 5-6 mice, 5 experiments. (7B). BMMφs from B6 and MRL/lpr mice were stimulated with IgG-ICs for 1 hr, or left untreated. Cells were stained for pSHIP1, CD11b (plasma membrane), and the nucleus, then visualized on a confocal microscope. Graphed is the quantification of pSHIP1 co-localized with CD11b of all cells imaged. n=15 cells, 3 mice, 2 experiments. (7C). The localization of pSHIP1 with lipid rafts (cholera toxin; Ctx) in B6, or MRL/lpr BMMφs using confocal microscopy. Graphed is the quantification of pSHIP1 co-localized with Ctx of all cells imaged. n=50 cells, 3 mice, 3 experiments. (7D). B6 and MRL/lpr BMMφs were attached to glass-bottom Petri dishes and stimulated with IgG-ICs for 30 min. The localization of FcγRI and pSHIP1 was assessed by TIRF microscopy. Colocalization of pSHIP1 with IgG-ICs bound to FcγRI was quantified by the Mander's coefficient and graphed. n=2 mice, 2 experiments, >20 cells.

FIG. 8. Crosslinking FcγRI with FcγRIIb (SM-PEG (1, 2)) restores SHIP1 localization to lipid rafts. The localization of pSHIP1 with lipid rafts (cholera toxin; Ctx) in B6 or MRL/lpr BMMφs using confocal microscopy. The localization of pSHIP1 with Ctx when FcγRI and FcγRIIb are crosslinked with SM-PEG (1, 2). Graphed is the quantification of pSHIP1 co-localized with Ctx of all cells imaged. n=20-50 cells, 3 mice, 3 experiments.

FIGS. 9A-9C. Crosslinking FcγRI with FcγRIIb (SM-PEG (1, 2)) restores lysosome acidification, SHIP1 phosphorylation, and clearance of membrane bound nuclear antigens. (9A). BMMφs (left panel) or splenic CD11b+ myeloid cells from B6 and MRL/lpr (right panel) were stimulated with IgG-ICs±SM-PEG (1, 2) (5 μg/mL) or control SM-PEG (2.1) (5 μg/mL), and ratiometric flow cytometry was used to quantify the relative phagosomal pH. For internal control, cells were treated with concanamycin A (CMA) 2 hrs prior to IgG-ICs treatment (T₀+CMA) to inhibit lysosomal pH change. 2-7 mice, >2 experiments. (9B). BMMφs (left panel) or splenic CD11b+ myeloid cells (right panel) were stimulated with SM-PEG (1, 2) or control SM-PEG (2, 1), or left unstimulated for 30 min or 60 min. At each time points, fixed cells were analyzed for surface bound nucleosomes. 2 mice, 2 experiments. (9C). BMMφs were stimulated with IgG-ICs±SM-PEG(1, 2) (5 μg/mL) for 1 hr, then pAKT (Ser/Thr) and pSHIP1 levels were assessed by flow cytometry. Phospho-proteins were normalized to isotype controls and the levels of total protein. 4 mice, 4 experiments.

FIG. 10. Nuclear antigens accumulate on dendritic cells (DCs) and macrophages (MFs), T and B cells in MRL/lpr mice. Single cell suspension of splenocytes from B6 and MRL/lpr mice were stained for Sm and cell surface markers for DCs (CD11c), Mφs (CD11b), B cells (CD19), T cells (CD4). Fixed cells were analyzed by flow cytometry; Light gray line: Isotype control staining, Black line: Sm staining. Representative histograms from more than five experiments are shown (n=>20 mice).

FIGS. 11A-11C. Diminished lysosome acidification is evident in BMMφs and ex vivo Mφs, B cells, and DCs from MRL/lpr mice. (11A-11C). Splenocytes from B6 and MRL/lpr mice were treated with IgG-ICs for indicated time points and analyzed for relative pH using ratiometric flow cytometry for CD11b+ Myeloid cells (11A), CD19+ B cells (11B), and CD11c+ DCs (11C). As a control, cells were pre-treated with concanamycin A (CMA) 2 hr prior to the stimulation with IgG-ICs for 30 min in order to inhibit lysosomal pH change. n=3 mice, 3 experiments.

FIGS. 12A-12C. Lysosome defects are evident in multiple murine models of SLE. (12A-12B). Splenic CD11b+ myeloid cells from the indicated mice were stimulated with IgG-ICs and then assessed for lysosomal pH by ratiometric flow cytometry (12A), or assessed for ROS production (12B) at designated time points. (12C). BMMφs from the indicated mice were stimulated with fluorescent IgG-ICs. At the indicated time points, intact IgG-ICs that recycled to the cell membrane were enumerated by flow cytometry. n=2-4 mice, 3 experiments.

FIGS. 13A-13D. Hematopoietic cells from NOD mice accumulated nuclear antigens and insulin on the plasma membrane. (13A-13C) PBMCs from NOD mice with different blood glucose levels and B6 mice were assessed for the levels of surface bound Sm on myeloid cells (CD11+; 13A), B cells (CD19+; 13B) and T cells (CD3+, 13C) by flow cytometry; 2 exp, 2-6 mice. The MFI was normalized to the isotype control staining and relative to B6. (13D) Splenocytes from B6, MRL/lpr, or 3 week old, pre-diabetic (blood glucose levels <100 mg/dL) mice were assessed for the levels of surface bound insulin on Mφs (CD11b+ CD11c−), DCs (CD11b+ CD11c+), B (CD19+) and T cells (CD3+) by flow cytometry; 3 exp, 3-4 mice. The MFI was normalized to the isotype control staining and relative to B6.

FIG. 14. Human blood B cells from active but not inactive SLE patients accumulated DNA on the plasma membrane. Whole blood cells from healthy controls or SLE patients were stained for surface bound DNA using anti-human DNA antibody (33H11) and cell surface markers (CD19 for B cells, CD3 for T cells, and CD14 for monocytes). Samples were analyzed by flow cytometry. SLE patient samples were subdivided into active and inactive groups using SLEDAI scores (Active: SLEDAI score≥6, Inactive: SLEDAI score≤5). Each dot represents a single sample and the bar represents the median value. The numbers of subjects analyzed in each group are shown in the table.

FIGS. 15A-15B. Hematopoietic cells from SLE patients showed impaired lysosomal acidification and diminished SHIP1 phosphorylation. (15A) Whole blood cells from healthy controls or SLE patients experiencing active disease (SLEDAI score≥6) were treated with pre-made IgG-ICs composed of apoptotic debris and anti-nucleosome IgG. At 15 min, 30 min, 60 min, and 120 min time points, cells were stained with Lysosensor™ Yellow/Blue dye to measure lysosomal pH and cell surface markers (CD14 for Monocytes, CD11c for DCs and CD19 for B cells), and analyzed by flow cytometry. For an internal control, cells were treated with concanamycin A (CMA) 2 hrs prior to IgG-ICs treatment (T₀+CMA) to inhibit lysosomal pH change and provide a baseline reading. Relative pH was calculated by MFI of Blue dye/MFI of Yellow dye, then normalized to T0+CMA. Data were collected from six experiments (n=6 healthy controls and 6 SLE patients). (15B) Whole blood cells from healthy controls, SLE patients experiencing active disease (SLEDAI score≥6) were left untreated or treated with pre-made IgG-ICs composed of apoptotic debris and anti-nucleosome IgG. At 60 min time point, cells were fixed in 3% paraformaldehyde solution and stained for intracellular proteins (pSHIP1 or total SHIP1) and cell surface markers (CD14 for Monocytes, CD11c for DCs, and CD19 for B cells). Levels of phosphorylated proteins were calculated by dividing MFI of phosphorylated proteins by MFI of total proteins. The fold changes after IgG-IC treatment (Levels after IgG-IC treatment/Levels from untreated cells) were graphed. Data were collected from five experiments (n=5 healthy controls, 6 active SLE patients).

FIGS.16A-16D. Examples of bispecific antibody constructs that cross link FcγRI and FcγRIIb. Examples of bispecific multivalent antibodies. (16A) Fab-IgG is tetravalent and contains an intact IgG that reacts with FcγRI and Fab domains appended to the N-terminus of each heavy chain that react with FcγRIIB. (16B) A trivalent Fab (Tri-Fab) lacks an Fc region and is composed of two Fabs reacting with FcγRIIB and one Fab for FcγRI. (16C) IgG-Fab has Fab domains appended to the C-terminus of each heavy chain. These Fabs bind to FcγRI. The intact IgG reacts with FcγRIIB. (16D) Fab-bsIgG is a bi-specific IgG that has one Fab of intact IgG recognizes FcγRI and the other Fab for FcγRIIB. In addition, it has an appended Fab on one heavy chain that binds FcγRIIb. All constructs encode the same antibodies as SM-PEG[1, 2].

FIGS. 17A-17B. Like SM-PEG(1, 2), Tri-Fab, IgG1Fab and Fab-bsIgG1 restore lysosome acidification and clear IgG-ICs from the plasma membrane. (17A) Splenocytes from B6 and MRL/lpr mice were treated with pre-made IgG-ICs composed of apoptotic debris and anti-nucleosome IgG (B6, MRL/lpr). MRL/lpr cells were stimulated with SM-PEG (2, 1), SM-PEG (1, 2), TriFab, IgG1Fab, or Fab-bsIgG1 simultaneously with IgG-ICs (MRL/lpr+SM-PEG (2, 1), MRL/lpr+SM-PEG (1, 2), MRL/lpr+TriFab, MRL/lpr+IgG1Fab, MRL/lpr+Fab-bsIgG1). At 30 min, 60 min, and 120 min time points, cells were stained with Lysosensor™ Yellow/Blue dye to measure lysosomal pH and cell surface marker (CD11b), and analyzed by ratiometric flow cytometry. For internal control, cells were treated with concanamycin A (CMA) 2 hrs prior to IgG-ICs treatment (T₀+CMA) to inhibit lysosomal pH change. (17B) Cells were treated as panel A. At 0, 30, 60 min time points, cells were stained for surface bound nucleosomes and cell surface markers (CD11b). Samples were analyzed by flow cytometry. Data (panel A and B) were collected from two experiments (n=2 mice).

FIG. 18. Model of how FcγRI-bound IgG-ICs recycle and the downstream consequences of diminished lysosomal acidification in MRL/lpr mice. In lupus-prone mice, early formation of IgG-ICs (composed antibodies to nuclear self-antigens and apoptotic debris) heightens the activation of FcγRI. This induces defective lysosomal maturation and acidification causing undegraded cargo to be recycled back to plasma membrane. This promotes the accumulation of FcγR-bound IgG-ICs on the plasma membrane perpetuating chronic FcγRI signaling and inducing a vicious cycle. In addition, defects in the maturation of lysosomes prolongs activation of lysosomal TLRs, and promotes leakage of cargo into the cytosol, activating cytosolic sensors such as AIM2 and TRIM21. This heightens the production of downstream cytokines including BAFF and type I IFNs.

FIG. 19. Defects in pSHIP1 are evident in MRL/lpr mice. Cell lysates (1−1.5×10⁶ cells) from unstimulated murine MFs were quantitated by immunoblot for the indicated proteins. Representative blots and densitometry quantification of multiple experiments are presented. Error bars=SEM. Student t test, *p=0.05, **p=0.01, ***p=0.001.

FIG. 20. Peripheral blood mononuclear cells from SLE patients experiencing active disease accumulate nuclear antigens and IgG on plasma membrane. Whole blood cells form healthy controls (HC) or SLE patients (SLE) with SLEDAI score≥6 or≤5 were analyzed for surface bound DNA (33H11) by flow cytometry. Data are shown as MFI values of DNA normalized to isotype control antibody staining. n=15 patients.

FIG. 21. Monocytes from the peripheral blood of MRL/lpr mice fail to acidify. PBMCs were stimulated with IgG-ICs and the relative lysosomal pH was quantitated using ratiometric flow cytometry. Only cells that fell within the monocyte gate and were negative for markers not expressed by monocytes (CD19, CD4, CD8, CD11c, Gr-1) were assessed. n=2 mice.

FIGS. 22A-22B. Longitudinal analysis comparing SLEDAI score and the accumulation of DNA on plasma membrane. SLE patients with active and inactive disease were followed longitudinally for the levels of DNA on the surface of B cells and monocytes. (22A). Examples of active patients who showed congruency or dissimilarity between SLEDAI scores and the levels of accumulated DNA on monocytes or inactive patients are shown. (22B). Summary of monocytes and B cells data from all patients analyzed were shown in the table.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” (and grammatical variants) means that the scope of a claim is to be interpreted to encompass the specified materials or steps recited in the claim, “and those that do not materially affect the basic and novel characteristic(s)” of the claimed invention. Thus, the term “consisting essentially of” when used in a claim of this invention is not intended to be interpreted to be equivalent to “comprising.”

In one embodiment, the present invention provides a synthetic molecule comprising at least one of a first immunoglobulin fragment antigen binding region (Fab1) and at least one of a second fragment antigen binding region (Fab2), wherein Fab1 comprises a first heavy chain comprising a variable region (first V_{H}), and a first light chain comprising a variable region (first V_{L}), wherein the first V_{H} and the first V_{L} form a first antigen binding site specific for an activating Fc receptor or Fc receptor-like molecule, and wherein Fab2 comprises a second heavy chain comprising a variable region (second V_{H}), and a second light chain comprising a variable region (second V_{L}), and the second V_{H} and the second V_{L} form a second antigen binding site specific for an inhibitory Fc receptor or Fc receptor-like molecule. In some embodiments, the synthetic molecule can also comprise at least one of a third fragment antigen binding region (Fab3), wherein Fab3 comprises a third heavy chain comprising a variable region (third V_{H}) and a third light chain comprising a variable region (third V_{L}), wherein the third V_{H} and the third V_{L} form a third antigen binding site specific for an inhibitory Fc receptor or Fc receptor-like molecule. In some embodiments, the synthetic molecule can also comprise at least one of a fourth fragment antigen binding region (Fab4), wherein Fab4 comprises a fourth heavy chain comprising a variable region (fourth V_{H}) and a fourth light chain comprising a variable region (fourth V_{L}), wherein the fourth V_{H} and the fourth V_{L} form a fourth antigen binding site specific for an activating Fc receptor or Fc receptor-like molecule.

As used herein, a “synthetic molecule” refers to a molecule that has been synthesized at the nucleic acid or amino acid level and/or modified via cleavage, chemical modification, fusion, mutation, etc. to result in a molecule that is different than a molecule found in nature.

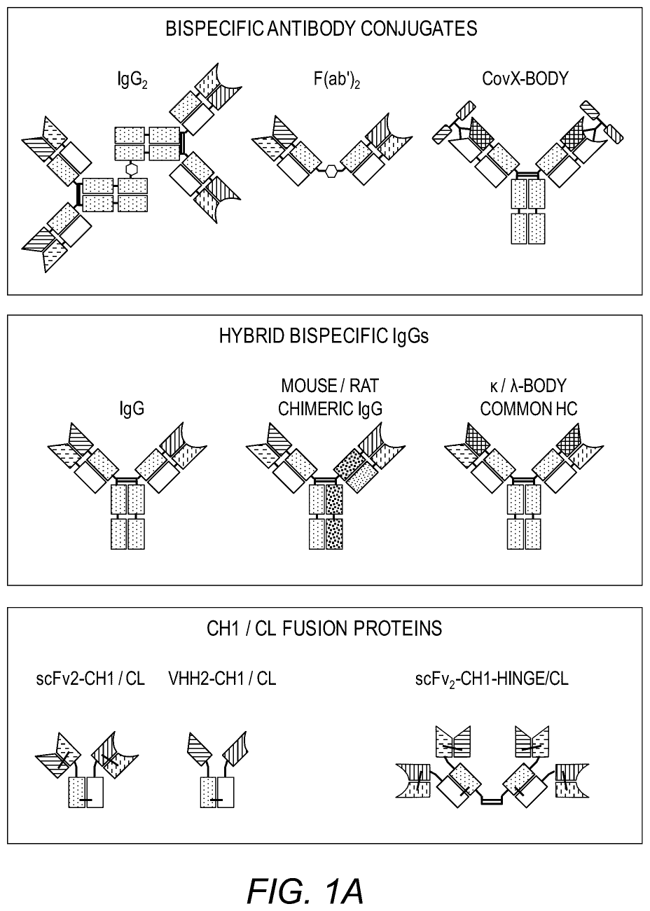

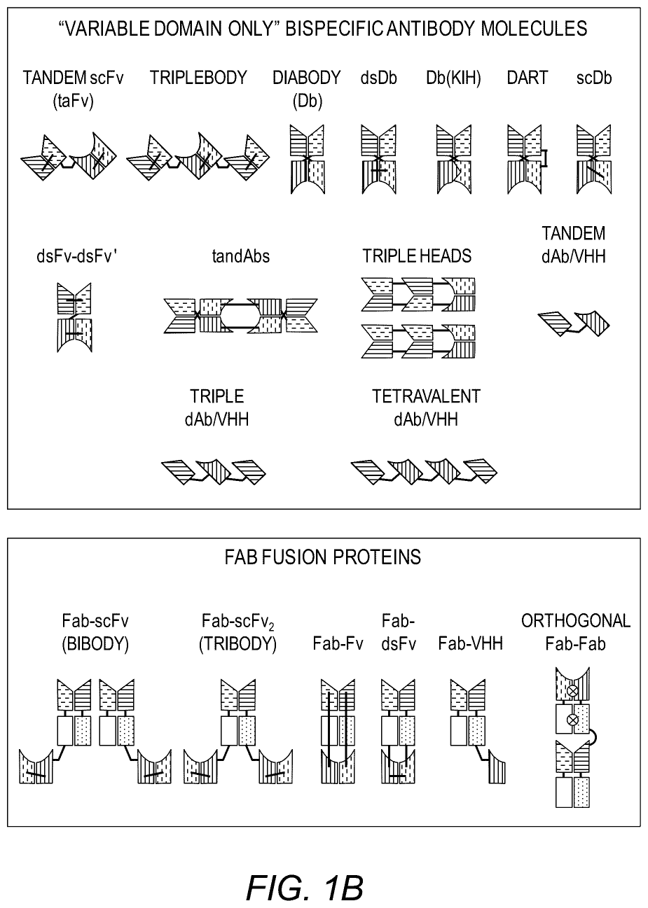

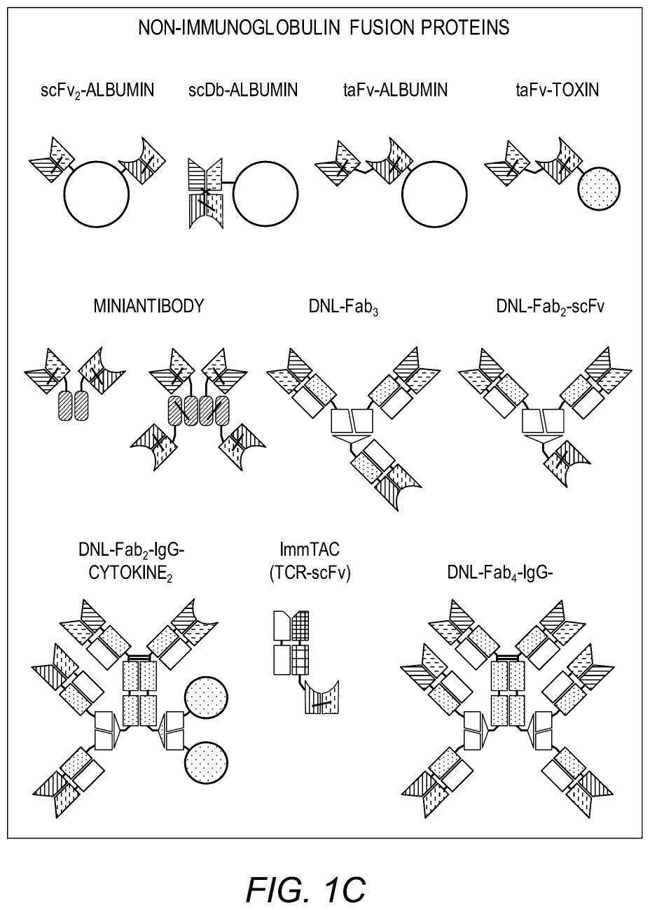

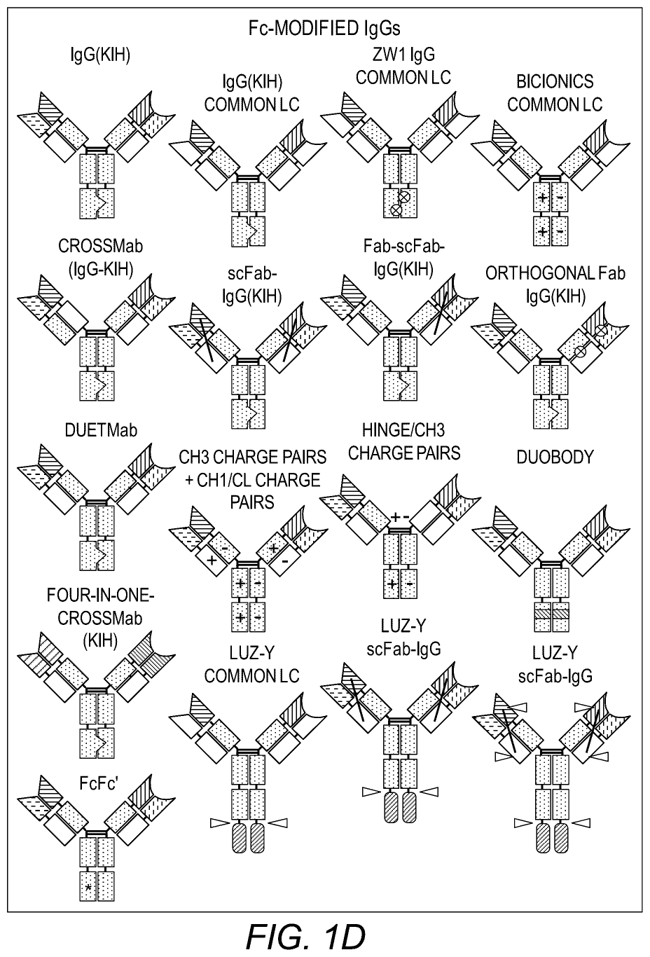

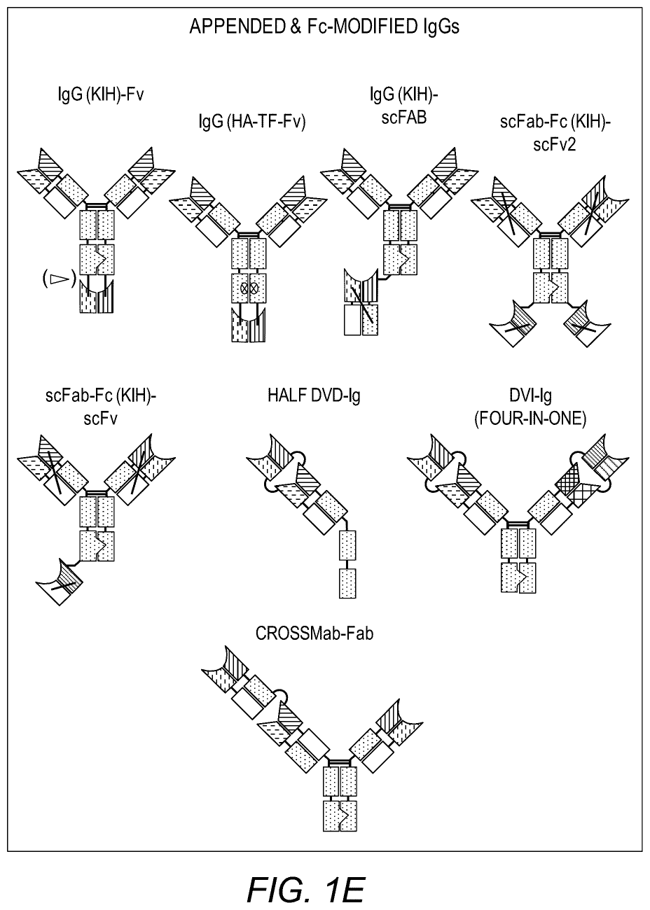

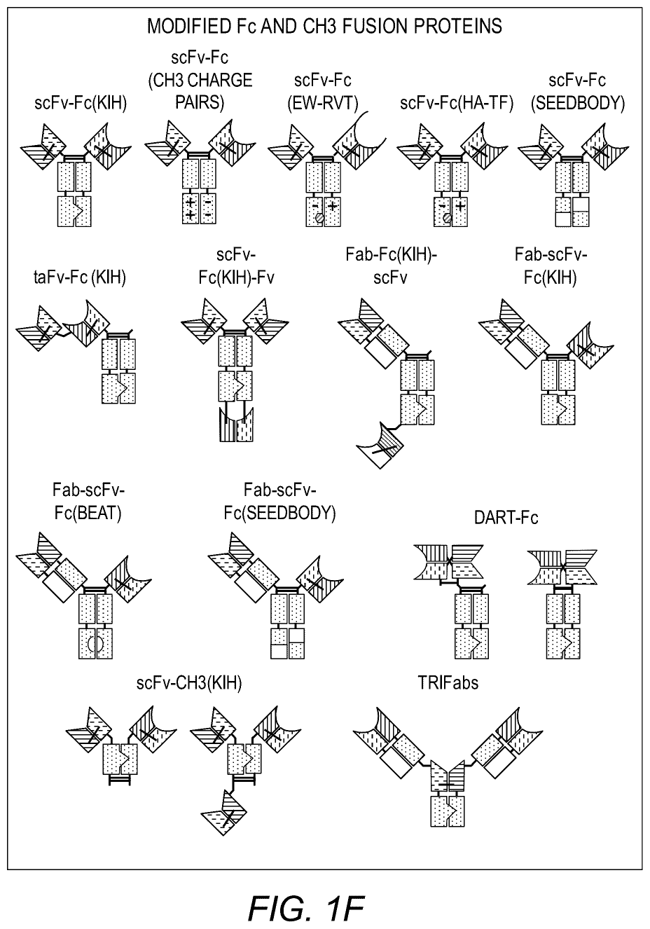

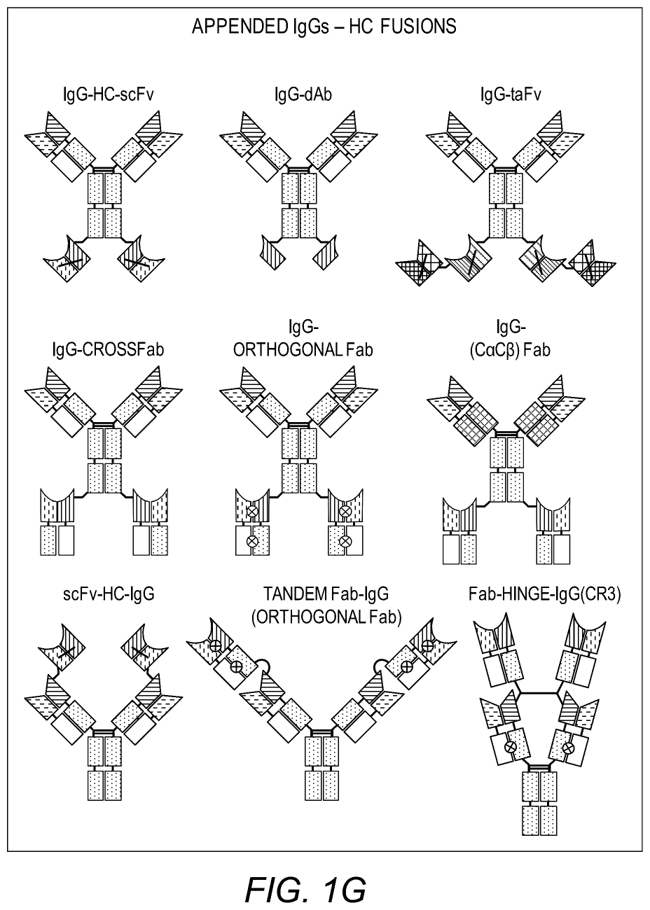

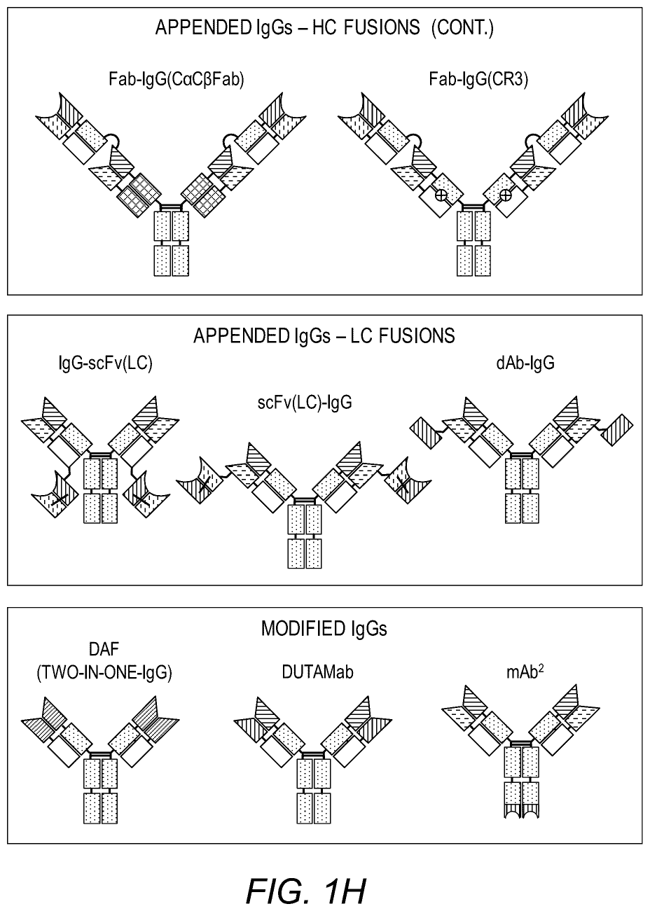

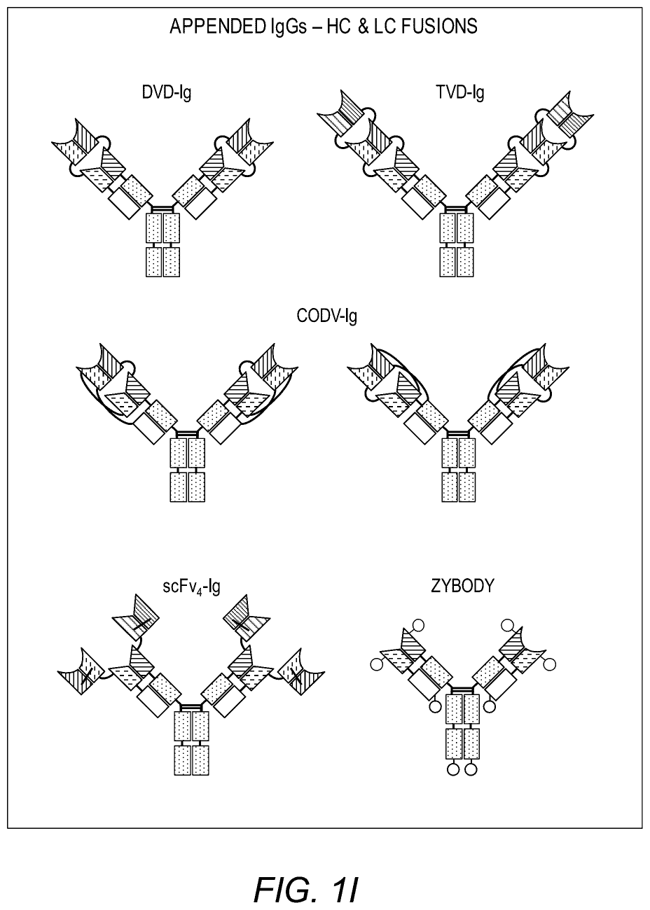

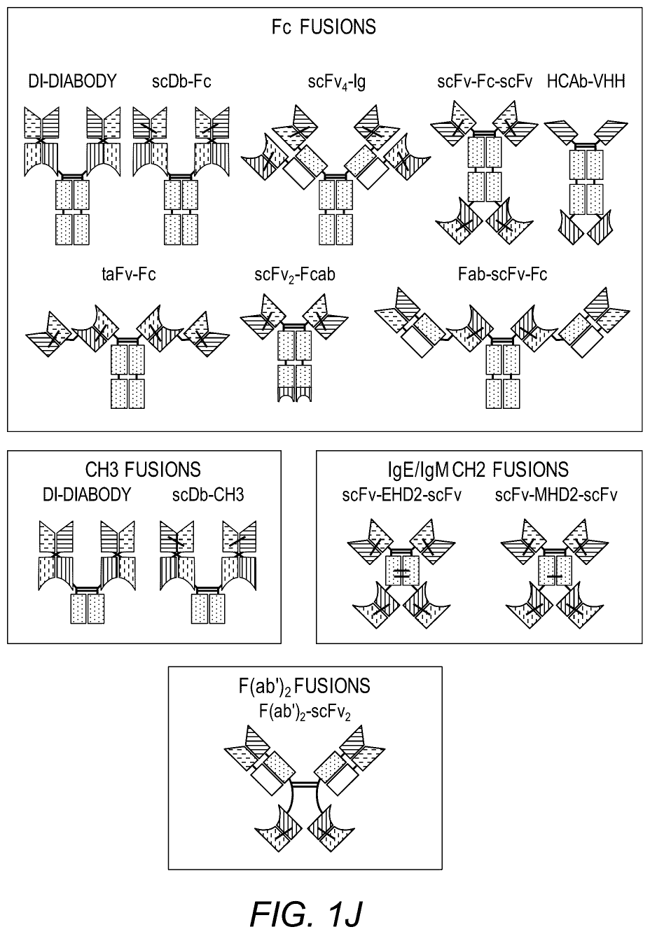

The synthetic molecules can have any configuration and orientation and any multiples of the components of the synthetic molecules. Nonlimiting examples of configurations in which the synthetic molecule of this invention can be present are provided in FIGS. 1A-1J (see Brinkmann and Kontermann “The making of bispecific antibodies” MABS9(2):182-212 (2017).

Nonlimiting examples of an activating Fc receptor or Fc receptor-like molecule include FcγRI, FcγRIIa, FcγRIIc, FcγRIIIA, FcγRIV, FcεRI, and leukocyte immunoglobulin-like receptor A (LILRA).

In some embodiments, Fab 1 is specific for FcγRI or FcγRIIa.

Nonlimiting examples of an inhibitory Fc receptor or Fc receptor-like molecule include FcγRIIb and leukocyte immunoglobulin receptor B (LILRB).

In some embodiments, Fab2 is specific for FcγRIIb.

In some embodiments, Fab3 is specific for FcγRIIb.

In some embodiments, Fab4 is specific for FcγRI or FcγRIIa.

In additional embodiments of this invention, the synthetic molecule described above can further comprise at least one immunoglobulin constant region that comprises at least one heavy chain (C_{H}) and/or at least one light chain (C_{L}).

In some embodiments, the synthetic molecule can comprise at least one Fab3 linked at the carboxy terminus and/or the amino terminus to at least one Fab1 and/or Fab2 at the carboxy terminus and/or the amino terminus of Fab1 and/or Fab2, and/or to at least one C_{H} and/or C_{L} at the carboxy terminus and/or the amino terminus of C_{H} and/or C_{L}, in any combination and in any number of multiples of Fab1, Fab2, Fab3, Fab4, C_{H} and C_{L}.

In some embodiments, the synthetic molecule can comprise at least one Fab4 linked at the carboxy terminus and/or the amino terminus to at least one Fab1 and/or Fab2 and/or Fab3 at the carboxy terminus and/or the amino terminus of Fab1 and/or Fab2 and/or Fab3, and/or to at least one C_{H} and/or C_{L} at the carboxy terminus and/or the amino terminus of C_{H} and/or C_{L}, in any combination and in any number of multiples of Fab1, Fab2, Fab3, Fab4, C_{H} and C_{L}.

In some embodiments, the synthetic molecule can comprise at least one Fab1 and/or Fab2 linked to one another at the carboxy terminus and/or the amino terminus of Fab1 and/or Fab2, in any combination and in any number of multiples of Fab1 and Fab2.

In some embodiments, the synthetic molecule of this invention can comprise at least one immunoglobulin Fc region.

In some embodiments, the synthetic molecule of this invention can comprise at least one Fab1, at least one Fab2 and at least one Fab3, wherein at least one Fab1 and/or at least one Fab2 is linked at the carboxy terminus and/or the amino terminus to at least one Fc region at the carboxy terminus and/or the amino terminus of Fc and/or at least one Fab3 is linked at the carboxy terminus and/or amino terminus to at least one Fc region at the carboxy terminus and/or amino terminus of Fc and/or to the carboxy terminus and/or the amino terminus of Fab1 and/or Fab2, in any combination and in any number of multiples of Fab1, Fab2, Fab3, and Fc.

In some embodiments, the synthetic molecule can comprise at least one Fab1, at least one Fab2, at least one Fab3 and at least one Fab 4, wherein at least one Fab1 and/or at least one Fab2 is linked at the carboxy terminus and/or amino terminus to at least one Fc region at the carboxy terminus and/or amino terminus of Fc and/or at least one Fab3 is linked at the carboxy terminus and/or amino terminus to at least one Fc region at the carboxy terminus and/or amino terminus of Fc and/or to at least one Fab1 and/or Fab2 at the carboxy terminus and/or amino terminus of Fab1 and/or Fab2, and/or at least one Fab4 is linked at the carboxy terminus and/or amino terminus to at least one Fc region at the carboxy terminus and/or amino terminus of Fc and/or at the carboxy terminus and/or the amino terminus of at least one Fab1 and/or Fab2 at the carboxy terminus and/or amino terminus of Fab1 and/or Fab2, and/or at least one Fab4 is linked at the carboxy terminus and/or the amino terminus to at least one Fab3 at the carboxy terminus and/or the amino terminus of Fab3, in any combination and in any number of multiples of Fab1, Fab2, Fab3, Fab4 and Fc.

It is to be understood that the synthetic molecule of this invention can comprise any combination of Fab1, Fab2, Fab3, Fab4, C_{H}, C_{L} and/or Fc, linked to one another in any combination and in any multiples of Fab1, Fab2, Fab3, Fab4, C_{H}, C_{L} and/or Fc. The Fab1, Fab2, Fab3, Fab4, C_{H}, C_{L} and/or Fc moieties can be linked to one another via a linker molecule. Nonlimiting examples of a linker molecule of this invention include a peptide (e.g., comprising 3-12 amino acids), a glycine-serine (G₄S)ₙ linker that may or may not contain additional amino acids such as Thr, Ala and/or Lys and Glu, a glycine linker of repeating glycine resides (6-8), a disulfide linker, a helical linker, a flexible linker (e.g., from Trichoderma Reesicelobiohydrolase I), a linker comprising the amino acid sequence KESGSVSSEQLAQFRSLD (SEQ ID NO:1), a linker comprising the amino acid sequence EGKSSGSGSESKST (SEQ ID NO:2), a linker comprising the amino acid sequence GSAGSAAGSGEF (SEQ ID NO:3), an alpha helix linker such as (EAAAK)ₙ (SEQ ID NO:4), a rigid linker such as A(EAAAK)ₙA (SEQ ID NO:5) (n=2-5) or (XP)ₙ, with X designating any amino acid, preferably Ala, Lys, or Glu, a hinge sequence (e.g., from shark), a short alanine linker, a hydrophilic linker, a chelating agent capable of forming a linkage, a sequence from human muscle aldolase, and any combination thereof. Additional examples of linkers that can be used in the present invention can be found at www.ibi.vu.nl/programs/linkerdbwww/.

Any of the synthetic molecules of this invention can further comprise a nonimmunoglobulin molecule, in any combination and in any multiples of any Fab1, Fab2, Fab3, Fab4, CH, CL and/or Fc that may be present. Nonlimiting examples of a nonimmunoglobulin molecule include a drug, a toxin, a therapeutic agent, a molecule to extend plasma half-life (i.e., human serum albumin or transferrin), cell penetrating peptides, ligands for targeting a functional protein (i.e., cytokine, chemokine, growth factors), or another receptor such a tumor recognizing receptor (i.e., T cell receptor).

In some embodiments, the nonimmunoglobulin molecule can be an engineered protein scaffold, which is made up of natural protein structures engineered to create a binding site for a specific target. Nonlimiting examples include affibodies (protein A domains), Kunitz domains, monobodies or adnectins, anticalins/lipocalins, DARPins/ankyrin repeat domains, avimers, knottin peptides, microbodies, peptide aptamer, affilin, tetranectin, CTLA-4, tendamistat, FN3, designed AR or TPR proteins, Zn fingers, pVIII, avian pancreatic polypeptide, GCN4, WW domain, SH3 domains, PDZ domains, TEM-1 beta-lactamase, GFP, thioredoxin, Staphylococcal nuclease, PHD-finger, CI-2, BPTI, APPI, HPSTI, ecotin, LACI-D1, LDTI, MTI-II, scorpion toxins, insect defensin A peptide, EETI-II, Min-23, CBD, PBP, gamma-crystallin, ubiquitin, transferrin, C-type lectin-like domain and any combination thereof, as well as any other engineered protein scaffold now known or later developed.

In some embodiments, the present invention provides a composition comprising a first synthetic molecule comprising at least one Fab1 specific for FcγRI and at least one Fab2 specific for FcγRIIb and a second synthetic molecule comprising at least one Fab1 specific for FcγRIIa and at least one Fab2 specific for FcγRIIb.

In some embodiments, the first synthetic molecule of said composition can comprise at least one Fab3 specific for FcγRIIb and/or the second synthetic molecule comprises at least one Fab3 specific for FcγRIIb.

In some embodiments of said composition, the first synthetic molecule can further comprise at least one Fab4 specific for FcγRI or FcγRIIa and/or the second synthetic molecule further comprises at least one Fab4 specific for FcγRI or FcγRIIa.

In some embodiments of said composition the first synthetic molecule and/or the second synthetic molecule can further comprise at least one immunoglobulin constant region that comprises at least one heavy chain (C_{H}) and/or at least one light chain (C_{L}) and/or the first synthetic molecule and/or the second synthetic molecule can further comprise at least one immunoglobulin Fc region.

Thus, it is to be understood that in said composition, the first synthetic molecule can comprise at least one Fab1 and at least one Fab2 and can further comprise at least one Fab3 and/or at least one Fab4 and/or at least one C_{H} and/or at least one C_{L} and/or at least one Fc, in any combination and in any number of multiplicities of Fab1, Fab2, Fab3, Fab4, C_{H}, C_{L} and Fc, when present in the synthetic molecule.

Further understood is that in said composition the second synthetic molecule can comprise at least one Fab1 and at least one Fab2 and can further comprise at least one Fab3 and/or at least one Fab4 and/or at least one C_{H} and/or at least one C_{L} and/or at least one Fc, in any combination and in any number of multiplicities of Fab1, Fab2, Fab3, Fab4, C_{H}, C_{L} and Fc, when present in the synthetic molecule.

Thus, in some embodiments of said composition, the first synthetic molecule and/or the second synthetic molecule can comprise at least one immunoglobulin constant region that comprises at least one heavy chain (C_{H}) and/or at least one light chain (C_{L}), and/or wherein the first synthetic molecule and/or the second synthetic molecule comprises at least one immunoglobulin Fc region.

In some embodiments of said composition, the first synthetic molecule and the second synthetic molecule can be linked (e.g., by one or more linker molecules of this invention) at any one or more of any Fab1, Fab2, Fab3, Fab4, C_{H}, C_{L} and/or Fc that may be present in either the first and/or second synthetic molecule, in any combination and in any multiplicity of first synthetic molecules and second synthetic molecules.

The phrase “one of more” as used herein is to be interpreted to mean 1, 2, 3, 4, 5, 6, 7, 8, 9, 10, 11, 12, 13, 14, 15, 16, 17, 18, 19, 20, 21, 22, 23, 24, 25, 50, 75, 100, etc.

In one embodiment, the present invention provides a synthetic molecule comprising at least one (e.g., 1, 2, 3, 4, 5, 6, 7, 8, 9, 10, etc.) of a first immunoglobulin fragment antigen binding region (Fab1) and at least one (e.g., 1, 2, 3, 4, 5, 6, 7, 8, 9, 10, etc.) of a second fragment antigen binding region (Fab2), wherein Fab1 comprises a first heavy chain comprising a variable region (first V_{H}), and a first light chain comprising a variable region (first V_{L}), wherein the first V_{H} and the first V_{L} form a first antigen binding site specific for FcγRI or FcγRIIa, and wherein Fab2 comprises a second heavy chain comprising a variable region (second V_{H}), and a second light chain comprising a variable region (second V_{L}), and the second V_{H} and the second V_{L} form a second antigen binding site specific for FcγRIIb.

In some embodiments, the synthetic molecule described above can further comprise at least one (e.g., 1, 2, 3, 4, 5, 6, 7, 8, 9, 10, etc.) of a third fragment antigen binding region (Fab3), wherein Fab3 comprises a third heavy chain comprising a variable region (third V_{H}) and a third light chain comprising a variable region (third V_{L}), wherein the third V_{H} and the third V_{L} form a third antigen binding site specific for FcγRIIb.

In some embodiments, the synthetic molecule of this invention comprising Fab1, Fab2 and/or Fab3 can further comprise at least one (e.g., 1, 2, 3, 4, 5, 6, 7, 8, 9, 10, etc.) of a fourth fragment antigen binding region (Fab4), wherein Fab4 comprises a fourth heavy chain comprising a variable region (fourth V_{H}) and a fourth light chain comprising a variable region (fourth V_{L}), wherein the fourth V_{H} and the fourth V_{L} form a fourth antigen binding site specific for FcγRI or FcγRIIa.

In some embodiments, the synthetic molecule of this invention can comprise, consist essentially of or consist of Fab1, Fab2, Fab3 and/or Fab4 in any combination and in any orientation and in any number of multiples of Fab1, Fab2, Fab3 and/or Fab4 (e.g., 1, 2, 3, 4, 5, 6, 7, 8, 9, 10, etc. of Fab1; 1, 2, 3, 4, 5, 6, 7, 8, 9, 10, etc. of Fab2; 1, 2, 3, 4, 5, 6, 7, 8, 9, 10, etc. of Fab3; and/or 1, 2, 3, 4, 5, 6, 7, 8, 9, 10, etc. of Fab4). Nonlimiting examples of embodiments of this invention include a synthetic molecule comprising Fab1, a synthetic molecule comprising Fab2, a synthetic molecule comprising Fab3, a synthetic molecule comprising Fab4, a synthetic molecule comprising Fab1 and Fab2, a synthetic molecule comprising Fab1 and Fab3, a synthetic molecule comprising Fab1 and Fab4, a synthetic molecule comprising Fab2 and Fab3, a synthetic molecule comprising Fab2 and Fab4, a synthetic molecule comprising Fab3 and Fab4, a synthetic molecule comprising Fab1, Fab2 and Fab3, a synthetic molecule comprising Fab1, Fab2 and Fab4, a synthetic molecule comprising Fab1, Fab3, and Fab4, a synthetic molecule comprising Fab2,Fab3, Fab4, and a synthetic molecule comprising Fab1, Fab2, Fab3 and Fab4, wherein Fab1, Fab2, Fab3 and Fab4, when present in a given embodiment, can be present singly or in any number of multiples and in any combination of multiples.

In further embodiments, any of the synthetic molecules of this invention comprising any of at least one of Fab1, Fab2 Fab3 and/or Fab4 can further comprise at least one immunoglobulin constant region that comprises at least one (e.g., 1, 2, 3, 4, 5, 6, 7, 8, 9, 10, etc.) heavy chain (C_{H}) and/or at least one (e.g., 1, 2, 3, 4, 5, 6, 7, 8, 9, 10, etc.) light chain (C_{L}).

In some embodiments, the present invention includes a synthetic molecule of this invention, wherein at least one Fab3 is linked at the carboxy terminus and/or the amino terminus to at least one Fab1 and/or Fab2 at the carboxy terminus and/or the amino terminus of Fab1 and/or Fab2 and/or to at least one C_{H} and/or C_{L} at the carboxy terminus and/or the amino terminus of C_{H} and/or C_{L}, in any combination.

In some embodiments, the present invention includes a synthetic molecule of this invention, wherein at least one Fab3 is linked at the carboxy terminus and/or the amino terminus to at least one Fab1 and/or Fab2 at the carboxy terminus and/or the amino terminus of Fab1 and/or Fab2 and/or to at least one C_{H} and/or C_{L} at the carboxy terminus and/or the amino terminus of C_{H} and/or C_{L}, in any combination.

In some embodiments, the present invention includes a synthetic molecule of this invention, wherein at least one Fab4 is linked at the carboxy terminus and/or the amino terminus to at least one Fab1 and/or Fab2 and/or Fab3 at the carboxy terminus and/or the amino terminus of Fab1 and/or Fab2 and/or Fab3 and/or to at least one C_{H} and/or C_{L} at the carboxy terminus and/or the amino terminus of C_{H} and/or C_{L}, in any combination.

In some embodiments, the present invention includes a synthetic molecule of this invention, wherein at least one (e.g., 1, 2, 3, 4, 5, 6, 7, 8, 9, 10, etc.) Fab1 and/or at least one (e.g., 1, 2, 3, 4, 5, 6, 7, 8, 9, 10, etc.) Fab2 are linked to one another at the carboxy terminus and/or the amino terminus of Fab1 and/or Fab2, in any combination.

In further embodiments, any of the synthetic molecules of this invention can comprise at least one (e.g., 1, 2, 3, 4, 5, 6, 7, 8, 9, 10, etc.) immunoglobulin Fc region.

In some embodiments, the present invention includes a synthetic molecule of this invention comprising at least one Fab1, Fab2 and Fab3, wherein at least one Fab1 and/or Fab2 is linked at the carboxy terminus and/or the amino terminus to at least one Fc region at the carboxy terminus and/or the amino terminus of Fc and/or at least one Fab3 is linked at the carboxy terminus and/or amino terminus to at least one Fc region at the carboxy terminus and/or amino terminus of Fc and/or to the carboxy terminus and/or the amino terminus of Fab1 and/or Fab2.

In some embodiments, the present invention includes a synthetic molecule comprising at least one Fab1, Fab2, Fab3 and Fab 4, wherein at least one Fab1 and/or Fab2 is linked at the carboxy terminus and/or amino terminus to at least one Fc region at the carboxy terminus and/or amino terminus of Fc and/or at least one Fab3 is linked at the carboxy terminus and/or amino terminus to at least one Fc region at the carboxy terminus and/or amino terminus of Fc and/or to at least one Fab1 and/or Fab2 at the carboxy terminus and/or amino terminus of Fab1 and/or Fab2, and/or at least one Fab4 is linked at the carboxy terminus and/or amino terminus to at least one Fc region at the carboxy terminus and/or amino terminus of Fc and/or at the carboxy terminus and/or the amino terminus of at least one Fab1 and/or Fab2 at the carboxy terminus and/or amino terminus of Fab1 and/or Fab2, and/or at least one Fab4 is linked at the carboxy terminus and/or the amino terminus to at least one Fab3 at the carboxy terminus and/or the amino terminus of Fab3.

It is understood that in the embodiments of this invention that any Fab1, Fab2, Fab3, Fab4, C_{H}, C_{L} and/or Fc, when present in a synthetic molecule of this invention can be linked to one another in any combination and in any configuration and in any number of multiples of Fab1, Fab2, Fab3, Fab4, C_{H}, C_{L} and/or Fc.

It is further understood that in the embodiments of this invention that any Fab1, Fab2, Fab3, Fab4, C_{H}, C_{L} and/or Fc can be linked to one another in any combination and in any configuration and in any number of multiples of Fab1, Fab2, Fab3, Fab4, C_{H}, C_{L} and/or Fc via a linker molecule of this invention.

In further embodiments of this invention, the synthetic molecule of this invention can comprise a nonimmunoglobulin molecule linked in any configuration, in any orientation and in any number of multiples to any Fab1, Fab2, Fab3, Fab4, C_{H}, C_{L} and/or Fc present in the synthetic molecule.

The present invention provides a synthetic molecule that can comprise, consist essentially of and/or consist of at least one Fab1 and at least one Fab2 and can further include at least one Fab3 and/or at least one Fab4. Said synthetic molecule can further comprise at least one C_{H} and/or at least one C_{L} and/or at least one Fc and/or at least one nonimmunoglobulin molecule, wherein Fab1, Fab2, Fab3, Fab4, C_{H}, C_{L}, Fc and any nonimmunoglobulin molecule can be linked (e.g., via a linker molecule) to one another in any combination and in any multiples of Fab1, Fab2, Fab3, Fab4, C_{H}, C_{L}, Fc and nonimmunoglobulin molecule of this invention.

Further provided herein is an isolated nucleic acid molecule that encodes the synthetic molecule of this invention. A nucleic acid molecule can be present in a vector, plasmid and/or any nucleic acid construct.

Also provided herein is an isolated cell or non-human organism that has been transformed and/or transfected with a nucleic acid molecule of this invention.

Additionally provided herein is a composition comprising the synthetic molecule of this invention in any combination and/or multiplicities thereof and/or a nucleic acid molecule of this invention. Said composition can comprise a pharmaceutically acceptable carrier, diluent and/or adjuvant.

The present invention also provides methods of using the synthetic molecules, nucleic acid molecules, cells and/or compositions of this invention.

Thus, in one embodiment, the present invention provides a method for treating an autoimmune disorder in a subject, comprising administering to the subject an effective amount of the synthetic molecule of this invention and/or the composition of this invention and/or the nucleic acid molecule of this invention and/or the cell of this invention.

Nonlimiting examples of an autoimmune disorder, immune related and/or inflammatory disease or disorder of this invention include systemic lupus erythematosus (SLE), sepsis; colitis; malignancies; arthritis, including, but not limited to, rheumatoid arthritis, osteoarthritis, juvenile chronic arthritis, and spondyloarthropathies; systemic sclerosis; idiopathic inflammatory myopathies; Sjögren's syndrome; systemic vasculitis; sarcoidosis; autoimmune hemolytic anemia; autoimmune thrombocytopenia; thyroiditis; diabetes; immune-mediated renal disease; demyelinating diseases of the central and peripheral nervous systems such as multiple sclerosis, idiopathic demyelinating polyneuropathy or Guillain-Barre syndrome, and chronic inflammatory demyelinating polyneuropathy; Alzheimer's disease; myocarditis; kidney disease; obesity; cardiovascular disease; hepatobiliary diseases such as infectious, autoimmune chronic active hepatitis, primary biliary cirrhosis, hepatitis, granulomatous hepatitis, and sclerosing cholangitis; inflammatory bowel disease; gluten-sensitive enteropathy; Whipple's disease; autoimmune or immune-mediated skin diseases including bullous skin diseases, erythema multiforme and contact dermatitis or hypersensitivity; psoriasis; allergic diseases such as asthma, allergic rhinitis, atopic dermatitis, food hypersensitivity and urticaria; immunologic diseases of the lung such as asthma, allergies, COPD (chronic obstructive pulmonary disease), eosinophilic pneumonias, idiopathic pulmonary fibrosis and hypersensitivity pneumonitis; transplantation associated diseases including graft rejection and graft-versus-host-disease; inflammation of the eye including but not limited to retinitis and uveitis; and any combination thereof.

In one embodiment, the present invention provides a method of treating systemic lupus erythematosus (SLE), comprising administering to the subject an effective amount of the synthetic molecule of this invention and/or the composition of this invention and/or the nucleic acid molecule of this invention and/or the host cell of this invention.

In one embodiment, the present invention provides a method of treating diabetes in a subject, comprising administering to the subject an effective amount of the synthetic molecule of this invention and/or the composition of this invention and/or the nucleic acid molecule of this invention and/or the host cell of this invention.

In one embodiment, the present invention provides a method of treating arthritis in a subject, comprising administering to the subject an effective amount of the synthetic molecule of this invention and/or the composition of this invention and/or the nucleic acid molecule of this invention and/or the host cell of this invention.

Any of the methods described herein can further comprise administering an immunostimulatory agent to the subject.

The present invention also provides a synthetic molecule, nucleic acid molecule, cell and/or composition of the present invention for use in therapy; e.g., for use in the treatment of an autoimmune disorder, SLE, diabetes and/or arthritis.

The present invention also provides a pharmaceutical composition comprising a synthetic molecule, nucleic acid molecule, cell and/or composition of the present invention and one or more pharmaceutically acceptable carriers, diluents or excipients.

Another embodiment of the present invention comprises use of a synthetic molecule, nucleic acid molecule, cell and/or composition of the present invention a synthetic molecule, nucleic acid molecule, cell and/or composition of the present invention of the present invention in the manufacture of a medicament for the treatment of a disorder; e.g., for the treatment of an autoimmune disorder, SLE, diabetes and/or arthritis.

### Definitions

Activating Fcγ receptors bind immunoglobulin Fc regions and promote activation signals via their immunoreceptor tyrosine-based activation motif (ITAM) located in the cytoplasmic tail” Activating Fcγ receptors include FcγRI, FcγRIII, FcγRIV in mice, and FcγRI, FcγRIIa, FcγRIIc, FcγRIIIA, FcγRIV in humans

Inhibitory or repressor Fcγ receptors bind immunoglobulin Fc regions and promote inhibitory signals via their immunoreceptor tyrosine-based inhibitory motif (ITIM) located in the cytoplasmic tail.

FcγRI is an activating Fc gamma receptor that belongs to the immunoglobulin superfamily and is involved in phagocytosis of opsonized microbes or apoptotic debris. Also known as CD64, FcγRI binds to IgG more strongly than other FcγRs.

FcγRIIa is known as CD32a and is an activating receptor found in humans but not mice that binds Fc regions of immunoglobulins of the IgG isotype.

FcγRIIb is known as CD32b and is an inhibitory receptor that binds immunoglobulin Fc regions of the IgG isotype.

Leukocyte immunoglobulin-like receptors (LILRs) are the human orthologs of PIRA (activating) and PIRB (repressive or inhibitory) in mice. LILRs function like FcγRs in terms of activating and repressing (inhibiting) cells.

Immunoglobulin fragment antigen binding region (Fab) is a region on an antibody that binds to an antigen or target molecule. It is composed of one constant and one variable domain of each of the heavy and the light chain

Immunoglobulin is a Y-shaped protein produced by activated B cells and plasma cells that is used by the immune system to neutralize pathogens such as pathogenic bacteria and viruses and can be produced in autoimmune disease and targets endogenous proteins or nucleic acids that are considered “self.”

Immunoglobulin Fc region (fragment crystallizable region) is the tail region of an antibody that interacts with cell surface Fc receptors and proteins of the complement system. The Fc region confers effector function to antibodies.

Immunoglobulin heavy chain is a polypeptide subunit of an immunoglobulin that pairs via disulfide linkages to an identical protein. It contains variable and constant regions that carry out unique functions.

Immunoglobulin light chain is a polypeptide subunit of an antibody. There are two types of light chains, kappa and lambda. Each antibody has two light chains that are identical.

Immunoglobulin constant region is the same for all immunoglobulins of the same class but differs between classes. The constant regions consists of CH1, CH2, CH3 for the heavy chain and CL for the light chain.

Immunoglobulin variable domain (V_{H}) is important for binding antigens or targets. Typically, the Vh domains pair with variable domains of the light chain and the resulting three dimensional structure confers a unique conformation that recognizes the target.

Linker molecule is a peptide sequence used to fuse, connect, link or join two or more protein domains together.

A “subject” of this invention includes any subject that is susceptible to the various diseases and/or disorders described herein. Nonlimiting examples of subjects of this invention include mammals, such as humans, nonhuman primates, domesticated mammals (e.g., dogs, cats, rabbits, guinea pigs, rats), livestock and agricultural mammals (e.g., horses, bovine, pigs, goats). In other embodiments, a subject may additionally be an animal such as a bird or reptile. Thus, in some embodiments, a subject can be any domestic, commercially or clinically valuable animal. In particular embodiments, the compositions and methods of this invention have utility in veterinary applications. Subjects may be male or female and may be any age including neonate, infant, juvenile, adolescent, adult, and geriatric subjects. In particular embodiments, the subject is a human. A human subject of this invention can be of any age, gender, race or ethnic group (e.g., Caucasian (white), Asian, African, black, African American, African European, Hispanic, Mideastern, etc.).

A “subject in need thereof” is a subject known to have, or suspected of having, diagnosed with, or at risk of having disease or disorder described herein. A subject of this invention can also include a subject not previously known or suspected to have a disease or disorder as described herein or in need of treatment for disease or disorder as described herein. For example, a subject of this invention can be administered the synthetic molecules, nucleic acid molecules, cells and/or compositions of this invention even if it is not known or suspected that the subject has a disease or disorder as described herein (e.g., prophylactically). A subject of this invention is also a subject known or believed to be at risk of developing a disease or disorder as described herein.

As used herein, the term “antibody” includes intact immunoglobulin molecules as well as active fragments thereof, such as Fab, F(ab')2, and Fc, which are capable of binding the epitopic determinant of an antigen (i.e., antigenic determinant). Antibodies that bind the polypeptides of this invention are prepared using intact polypeptides and/or fragments containing small peptides of interest as the immunizing antigen. The polypeptide or fragment used to immunize an animal can be derived from enzymatic cleavage, recombinant expression, isolation from biological materials, synthesis, etc., and can be conjugated to a carrier protein, if desired. Commonly used carriers that are chemically coupled to peptides and proteins for the production of antibody include, but are not limited to, bovine serum albumin, thyroglobulin and keyhole limpet hemocyanin. The coupled peptide or protein is then used to immunize a host animal (e.g., a mouse, rat, goat, sheep, human or rabbit). The polypeptide or peptide antigens can also be administered with an immunostimulatory agent, as described herein and as otherwise known in the art.

The terms “antibody” and “antibodies” as used herein refer to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, and/or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody (scFv) or bispecific antibody.

Techniques for the production of chimeric antibodies or humanized antibodies by splicing mouse antibody genes to human antibody genes to obtain a molecule with appropriate antigen specificity and biological activity can be used (Morrison et al. 1984. Proc. Natl. Acad. Sci. 81:6851-6855; Neuberger et al. 1984. Nature 312:604-608; Takeda et al. 1985. Nature 314:452-454). Alternatively, techniques described for the production of single chain antibodies can be adapted, using methods known in the art, to produce single chain antibodies specific for the polypeptides and/or fragments and/or epitopes of this invention. Antibodies with related specificity, but of distinct idiotypic composition, can be generated by chain shuffling from random combinatorial immunoglobulin libraries (Burton 1991. Proc. Natl. Acad. Sci. 88:11120-3).

Active antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

Monoclonal antibodies can be produced in a hybridoma cell line according to the technique of Kohler and Milstein (Nature 265:495-97 (1975)). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacterial cell such as E. coli by recombinant techniques known to those skilled in the art. See, e.g., W. Huse, (1989) Science 246:1275-81.

Antibodies can also be obtained by phage display techniques known in the art or by immunizing a heterologous host with a cell containing an epitope or immunogen of interest.

As used herein, the term “polypeptide” encompasses both peptides and proteins, unless indicated otherwise.

The terms “polypeptide,” “protein,” and “peptide” refer to a chain of covalently linked amino acids. In general, the term “peptide” can refer to shorter chains of amino acids (e.g., 2-50 amino acids); however, all three terms overlap with respect to the length of the amino acid chain. Polypeptides, proteins, and peptides may comprise naturally occurring amino acids, non-naturally occurring amino acids, or a combination of both. The polypeptides, proteins, and peptides may be isolated from sources (e.g., cells or tissues) in which they naturally occur, produced recombinantly in cells in vivo or in vitro or in a test tube in vitro, and/or synthesized chemically. Such techniques are known to those skilled in the art. See, e.g., Sambrook et al., Molecular Cloning: A Laboratory Manual 2nd Ed. (Cold Spring Harbor, NY, 1989); Ausubel et al. Current Protocols in Molecular Biology (Green Publishing Associates, Inc. and John Wiley & Sons, Inc., New York).

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical, or substantially identical, to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention according to the methods described herein, which are routine methods for testing activities of polypeptides, and/or according to any art-known and routine methods for identifying such activities. Such production and testing to identify biologically active fragments of the polypeptides described herein would be well within the scope of one of ordinary skill in the art and would be routine.

The term “therapeutically effective amount” or “effective amount,” as used herein, refers to that amount of a composition of this invention that imparts a modulating effect, which, for example, can be a beneficial effect, to a subject afflicted with a disorder, disease or cosmetic condition, including improvement in the disease or disorder of the subject (e.g., in one or more symptoms), delay or reduction in the progression of the disease or disorder, prevention or delay of the onset of the disease or disorder, and/or change in clinical parameters of the disease or disorder, as would be well known in the art. The effective amount will vary with the age, general condition of the subject, the severity of the disease, disorder or condition being treated, the particular agent or composition administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science and Practice of Pharmacy (20th ed. 2000)). For example, a therapeutically effective amount or effective amount can refer to the amount of a composition, compound, or agent that improves a disease or disorder in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Although individual needs may vary, the determination of optimal ranges for effective amounts of a composition of this invention is within the skill of the art. Human doses can also readily be extrapolated from animal studies (Katocs et al., Chapter 27 In: Remington's Pharmaceutical Sciences, 18.sup.th Ed., Gennaro, ed., Mack Publishing Co., Easton, Pa., 1990). Generally, the dosage required to provide an effective amount of a composition of this invention, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3 In: Goodman & Gilman's The Pharmacological Basis of Therapeutics, 9.sup.th Ed., Hardman et al., eds., McGraw-Hill, New York, N.Y., 1996).

Thus, the amount of the composition or particle of this invention needed to deliver a pharmaceutically effective dosage will vary based on such factors including but not limited to, the polymer solubility, the therapeutic loading capacity and efficiency, the toxicity levels of the polymer, the amount and type of bioactive material needed to effect the desired response, the subject's species, age, weight, and condition, the disease and its severity, the mode of administration, and the like. One skilled in the art would be able to determine the pharmaceutically effective dosage. In general, the amount of composition of this invention that could be administered by the delivery systems of the invention is from about 1 pg/day (or from about 0.5 mg/day) to more than about 100 g/day quantities, including any values within this range that are not specifically spelled out here.

The pharmaceutical compositions according to the present invention may be administered as a single dose or in multiple doses. The pharmaceutical compositions of the present invention may be administered either as individual therapeutic agents or in combination with other therapeutic agents, which when combined may be administered sequentially or simultaneously.

By the terms “treat,” “treating” or “treatment of” (or grammatically equivalent terms) it is meant that the severity of the subject's disease or disorder is reduced or at least partially improved or ameliorated and/or that some alleviation, mitigation or decrease in at least one clinical symptom is achieved and/or there is a delay in the progression of the disease or disorder, as would be well known in the art. Thus, in some embodiments, the terms “treat,” “treating” or “treatment of” refer only to therapeutic regimens. In other embodiments, the terms “treat,” “treating” or “treatment of” (or grammatically equivalent terms) refer to both prophylactic and therapeutic regimens.

The terms “prevent,” “preventing” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is delayed and/or is less than what would occur in the absence of the method of the present invention.

An “effective amount,” as used herein, refers to an amount that imparts a desired effect, which is optionally a therapeutic or prophylactic effect.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

Pharmaceutical compositions comprising the composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with, inter alia, a pharmaceutically acceptable carrier.

By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

Exemplary modes of administration of the compositions of this invention can include oral, rectal, intranodal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intraperitoneal, intradermal, intrapleural, intracerebral, intracranial, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). The most suitable route in any given case will depend on the nature and severity of the condition being treated and on the nature of the particular protein, peptide, fragment, nucleic acid and/or vector that is being used.

The compositions of the present invention may be administered to a subject in need of treatment prior to, during or after onset of the disease or disorder. Thus, the compositions of the present invention can be used to treat ongoing immune-related and/or inflammatory diseases or disorders or to prevent diseases or delay the development of immune-related and/or inflammatory diseases or disorders.

In some embodiments, an effective dose or effective amount can comprise one or more (e.g., two or three or four or more) doses of the composition of this invention at any time interval (e.g., hourly, daily, weekly, monthly, yearly, as needed) so as to achieve and/or maintain the desired therapeutic benefit.

In some embodiments, the term “bispecific antibody” refers to a molecule comprising an immunoglobulin G antibody (IgG) conjugated to multiple Fab specific for FcγRI or FcγRIIa and FcγRIIb or two single chain variable fragments (scFv). As referred to herein, a bispecific antibody of the present invention can comprise one scFv or Fab covalently linked to the carboxy-terminus of a HC of the IgG by a linker and the other scFv or Fab covalently linked to the carboxy-terminus of the other HC of the IgG by a linker. The IgG and scFvs or Fabs of a bispecific antibody of the present invention specifically bind different antigens. For example, in some embodiments, FcγRI and FcγRIIb can be the different antigens targeted by the bispecific antibody and in some embodiments, FcγRIIa and FcγRIIb can be the different antigens targeted by the bispecific antibody.

As used herein, the term “immunoglobulin G antibody” (IgG), refers to an immunoglobulin molecule comprised of four polypeptide chains; two heavy chains (HC) and two light chains (LC) interconnected by disulfide bonds. The amino-terminal portion of each of the four polypeptide chains includes a variable region of about 100-110 or more amino acids primarily responsible for antigen recognition. The carboxy-terminal portion of each of the four polypeptide chains defines a constant region primarily responsible for effector function. The carboxy-terminal portion of each heavy chain is covalently attached to one of the single chain variable fragments (scFv) or Fabs via a linker.

The light chains (LC) of the IgG are classified as kappa or lambda and characterized by a particular constant region as known in the art. The heavy chains (HC) of the IgG according to the present invention are classified as gamma, which defines the isotype (e.g., as an IgG). The isotype may be further divided into subclasses (e.g., IgG₁, IgG₂, IgG₃, and IgG₄). Each HC type is characterized by a particular constant region known in the art. Each HC is comprised of an N-terminal heavy chain variable region (“HCVR”) and a heavy chain constant region (CH). The CH for IgG is comprised of three domains (CH1, CH2, and CH3). Each light chain of the IgG is comprised of a light chain variable region (LCVR) and a light chain constant region (CL). The HCVR and LCVR regions can be further subdivided into regions of hypervariability, termed complementarity determining regions (CDRs), interspersed with regions that are more conserved, termed framework regions (FR). Each HCVR and LCVR of the IgGs is composed of three CDRs and four FRs, arranged from amino-terminus to carboxy-terminus in the following order: FR1-1, CDR1, FR1-2, CDR2, FR1-3, CDR3, FR1-4. Herein the 3 CDRs of the HC are referred to as “HCDR1, HCDR2 and HCDR3” and the 3 CDRs of the LC are referred to as “LCDR1, LCDR2 and LCDR3.” The CDRs contain most of the residues which form specific interactions with the antigen. The functional ability of an antibody to bind a particular antigen is largely influenced by the six CDRs.

The term “Fab” refers to polypeptide chains comprising a heavy chain variable region (HCVR) and a light chain variable region (LCVR).

The term “single chain variable fragment” (scFv) refers to polypeptide chains comprising a heavy chain variable region (HCVR) and a light chain variable region (LCVR) connected via a polypeptide linker. Additionally, the HCVR of each scFv can be: a) covalently linked, at its N-terminus, to the C-terminus of one HC of the IgG via a first polypeptide linker; and b) covalently linked, at its C-terminus, to the N-terminus of the LCVR of the same scFv via a second polypeptide linker. Further, each scFv can include a disulfide bond formed between a cysteine residue of HCVR and a cysteine residue of LCVR of the same polypeptide chain.

HCVR and LCVR of the Fabs can be further subdivided into regions of hypervariability, termed complementarity determining regions (CDRs), interspersed with regions that are more conserved, termed framework regions (FR). Each HCVR and LCVR of the IgGs is composed of three CDRs and four FRs, arranged from amino-terminus to carboxy-terminus in the following order: FR2-1, CDR4, FR2-2, CDR5, FR2-3, CDR6, FR2-4. The 3 CDRs of HCVR of the Fab are referred to as “HCDR4, HCDR5 and HCDR6” and the 3 CDRs of LCVR2 of the scFv are referred to as “LCDR4, LCDR5 and LCDR6.” The CDRs contain most of the residues which form specific interactions with the targets. The functional ability of a Fab to bind a particular target is largely influenced by the six CDRs.

The variable regions of each light/heavy chain pair of an IgG and Fab, respectively, form an antigen-binding site of the bispecific antibody. The IgG has two antigen binding sites which are the same (but are different than the antigen binding sites of appended Fabs) and each Fab has an antigen binding site (which is the same as the antigen binding site of the another Fab).

As used herein, the “antigen-binding portion” or “antigen-binding site” or “antigen-binding region” or “antigen-binding fragment” refers interchangeably to that portion of an IgG or Fab molecule, within the variable region, which contains the amino acid residues that interact with the target and confer to the bispecific antibody specificity and affinity for a target. This antibody portion includes the framework amino acid residues necessary to maintain the proper conformation of the target-binding residues. Preferably, the framework regions of the bispecific antibodies of the invention are of human origin or substantially of human origin.

A “parent antibody” or “parental antibody,” as used interchangeably herein, is an antibody encoded by an amino acid sequence which is used in the preparation of one of the synthetic molecules of this invention, for example through amino acid substitutions and structural alteration. The parent antibody may be a murine, chimeric, humanized or human antibody. Nonlimiting examples of a parent antibody of this invention include an antibody that specifically reacts with human FcγRI, an antibody that specifically reacts with human FcγRIIa, an antibody that specifically reacts with FcγRIIc an antibody that specifically reacts with FcγRIIIA, an antibody that specifically reacts with FcγRIV, an antibody that specifically reacts with FcεRI, an antibody that specifically reacts with human FcγRIIb, an antibody that specifically reacts with LIRA, and/or an antibody that specifically reacts with LIRB.

The terms “Kabat numbering” or “Kabat labeling” are used interchangeably herein. These terms, which are recognized in the art, refer to a system of numbering amino acid residues which are more variable (i.e., hypervariable) than other amino acid residues in the heavy and light chains variable regions of an antibody (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)).

The terms “North numbering” or “North labeling” are used interchangeably herein. These terms, which are recognized in the art, refer to a system of numbering amino acid residues which are more variable (i.e., hypervariable) than other amino acid residues in the heavy and light chains variable regions of an antibody and is based, at least in part, on affinity propagation clustering with a large number of crystal structures, as described in (North et al. “A New Clustering of Antibody CDR Loop Conformations” Journal of Molecular Biology 406:228-256 (2011).

The terms “patient,” “subject,” and “individual,” used interchangeably herein, refer to an animal, which can be a mammal and in some embodiments, can be a human. In certain embodiments, the subject, preferably a human, is further characterized with a disease or disorder or condition (e.g., an autoimmune disorder) that would benefit from treatment according to the methods described herein.

In another embodiment the subject, preferably a human, is further characterized as being at risk of developing a disorder, disease or condition that would benefit from treatment according to the methods of this invention.

In further embodiments, the present invention provides a synthetic molecule comprising an anti-FcγRI heavy chain variable region (UNC1_VH) comprising the amino acid sequence

(SEQ ID NO: 6)
QFQLVQSGPELKKPGETVKISCKASGYTFTTYGMSWVKQAPGKGLKWMG
WINTYSGMPTYGDDFKGRFAFSLETSASTAYLQINNLKNEDTATYFCAR
NWDDWYFDVWGTGTTVTVSS.

Also provided herein is a synthetic molecule comprising an anti-FcγRI light chain variable region (UNC1_VK) comprising the amino acid sequence

(SEQ ID NO: 7)
DIQMTQSPASLAASVGETITITCQASENIYFSLAWYQQKQGKSPQLLIY
NANTLKHGVPSRFSGSGSGTQYSMKINNLQPEDTATYFCKQPYGFPRTF
GGGTKLEIK.

Further provided herein is a synthetic molecule comprising an anti-FcγRIIb heavy chain variable region (UNC2_VH) comprising the amino acid sequence

(SEQ ID NO: 8)
EVQLQQSGGGLVQPGRSLKLSCAASGFTFSDYYMAWVRQAPTTGLEWVA
SISYDGGDTHYRDSVKGRFTISRDNAKSSLYLQMDSLRSEDTATYYCAT
ETTGIPTGVMDAWGQGVSVTVSS.

Additionally provided herein is a synthetic molecule comprising an anti-FcγRIIb light chain variable region (UNC2_VK)

(SEQ ID NO: 9)
DVQMTQSPYNLAASPGESVSINCKASESISKYLAWYLQKPGKANKLLMY
DGSTLQSGIPSRFSGSGSGTDFTLTIRSLEPEDFGLYYCQQHYEYPATF
GSGTKLEIK.

These amino acid sequences can be linked to one another in any configuration to assemble into a fragment antigen binding region (Fab) that crosslinks FcγRI and FcγRIIb.

Also provided herein are nucleic acid molecules that encode the amino acid sequences described herein. Said nucleic acid molecules of this invention can be present in a vector, plasmid and/or nucleic acid construct.

Expression vectors capable of directing expression of genes or coding sequences to which they are operably linked are well known in the art. Expression vectors can encode a signal peptide that facilitates secretion of the polypeptide(s) from a host cell. The signal peptide can be an immunoglobulin signal peptide or a heterologous signal peptide. A first polypeptide chain and a second polypeptide chain may be expressed independently from different promoters to which they are operably linked in one vector or, alternatively, the first and second polypeptide chains may be expressed independently from different promoters to which they are operably linked in two vectors—one expressing the first polypeptide chain and one expressing the second polypeptide chain.

A host cell includes cells stably or transiently transfected, transformed, transduced or infected with one or more expression vectors expressing a first polypeptide chain, a second polypeptide chain or both a first and a second polypeptide chain of the invention. Creation and isolation of host cell lines producing a synthetic molecule of the invention can be accomplished using standard techniques known in the art. Mammalian cells are preferred host cells for expression of said synthetic molecules. Nonlimiting examples of mammalian cells that can be used include HEK 293, NS0, DG-44, and CHO. In some embodiments, the synthetic molecules are secreted into the medium in which the host cells are cultured, from which the synthetic molecules can be recovered or purified.

The medium into which a synthetic molecule has been secreted may be purified by conventional techniques. For example, the medium may be applied to and eluted from a Protein A or G column using conventional methods. Soluble aggregate and multimers may be effectively removed by common techniques, including size exclusion, hydrophobic interaction, ion exchange, or hydroxyapatite chromatography. The product may be immediately frozen, for example at −70° C., or may be lyophilized.

In some instances, a method of producing a synthetic molecule of the present invention may result in the formation of diabodies. Diabodies are bivalent formations of scFv in which HCVR2 and LCVR2 regions are expressed on a single polypeptide chain, but instead of the variable domains pairing with complementary domains of the same polypeptide chain, the variable domains pair with complementary domains of the other polypeptide chain or a different molecule. For example, if the synthetic molecule comprises two first polypeptides (for example, 1A and 1B, where each of 1A and 1B comprise a HC, a scFv, L1 and L2), and two second polypeptides (for convenience, 2A and 2B, where each of 2A and 2B comprise a LC), HCVR2 of 1A pairs with complementary domains of LCVR2 of 1B instead of pairing with LCVR2 of 1A. As described herein, it may be beneficial to purify out diabodies from the synthetic molecules described above. Diabody content can be greater than 10% after cellular expression and can be reduced to less than 3% after purification.

### Therapeutic Uses

As used herein, “treatment” and/or “treating” are intended to refer to all processes wherein there may be a slowing, interrupting, arresting, controlling, or stopping of the progression of the disorders described herein, but does not necessarily indicate a total elimination of all disorder symptoms. Treatment includes administration of a synthetic molecule and/or composition of the present invention for treatment of a disease or condition in a mammal, particularly in a human, that would benefit from crosslinking of FcγRI and FcγRIIB and/or crosslinking of FcγRIIa and FcγRIIB, and includes: (a) inhibiting further progression of the disease, i.e., arresting its development; and/or (b) ameliorating the disease, i.e., causing regression of the disease or disorder or alleviating symptoms or complications thereof.

### Pharmaceutical Composition

A synthetic molecule of the invention can be incorporated into a pharmaceutical composition suitable for administration to a patient. A synthetic molecule of the invention may be administered to a patient alone or with a pharmaceutically acceptable carrier and/or diluent in single or multiple doses. Such pharmaceutical compositions are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions can be designed in accordance with conventional techniques disclosed in, e.g., Remington, The Science and Practice of Pharmacy, latest edition, Loyd V,, Ed., Pharmaceutical Press, which provides a compendium of formulation techniques as are generally known to practitioners. Suitable carriers for pharmaceutical compositions include any material which, when combined with a synthetic molecule of the invention, retains the molecule's activity and is non-reactive with the patient's immune system. A pharmaceutical composition of the present invention comprises one or more synthetic molecules of this invention and one or more pharmaceutically acceptable carriers, diluents or excipients.

A pharmaceutical composition comprising a synthetic molecule of the present invention can be administered to a patient at risk for or exhibiting diseases or disorders as described herein using standard administration techniques.

A pharmaceutical composition of the invention contains an “effective” or “therapeutically effective” amount, as used interchangeably herein, of a synthetic molecule of the invention. An effective amount refers to an amount necessary (at dosages and for periods of time and for the means of administration) to achieve the desired therapeutic result. An effective amount of the synthetic molecule may vary according to factors such as the disease state, age, sex, and weight of the individual, and the ability of the synthetic molecule to elicit a desired response in the individual. An effective amount is also one in which any toxic or detrimental effect of the synthetic molecule, are outweighed by the therapeutically beneficial effects.

### EXAMPLES

The present subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1

### Overview

The etiology of systemic lupus erythematosus (SLE) remains unknown despite a vast amount of research that identifies many receptors, genes, and signaling defects in dendritic cells (DCs), macrophages (Mφs), neutrophils, B and T cells. The lack of a clear mechanistic picture of SLE largely reflects a lack of understanding of the inter-relationships and order of events that initiate and exacerbate this autoimmune disease. Such gaps in our knowledge severely limit rationale design of therapies to treat SLE.

We recently identified a defect in lysosome function that promotes the accumulation of nuclear antigens (Smith; Sm) on the surface of multiple hematopoietic cells from several murine models of lupus. The defect is also evident in human SLE. The defect in lysosome function is biologically relevant in disease because it lies upstream of several hallmarks of SLE including cytokine secretion, autoantibody (autoAb) production, and end-organ disease. In this provisional patent application, we propose a therapy that restores lysosome function in myeloid cells, a cell type we show is dysregulated prior to the dysregulation of lymphoid cells. We summarize data identifying the lysosome defect and its consequences in SLE, then provide the rationale for our target molecules and the supporting evidence that the therapeutic reverses the defects in lysosome function and downstream events. The value of this technology is that the lysosome dysfunction appears to be an upstream “branch-point” that could simultaneously attenuate multiple downstream pathologies, antiquating several current SLE therapies.

### Background-Evidence for a Lysosome Defect in Lupus

Lysosomal maturation is a two-step process wherein phagocytosis induces assembly of the NADPH oxidase on the phagosomal membrane promoting the transfer of electrons to molecular oxygen. This produces superoxide and other reactive oxygen species (ROS). Termination of the oxidative burst and assembly of the V-ATPase acidifies the lysosome, activating hydrolytic enzymes that degrade internalized cargo. Thus, the lysosome acts much like the “gut” of the macrophage and plays a key role in disposal of waste internalized through phagocytosis. The defect we identified in SLE prevents lysosome maturation because the oxidative burst is not attenuated, and acidification of the lysosome is markedly reduced. Relevant to SLE is the inefficient disposal of dying cells (apoptotic cells), because dying cells progress to secondary necrotic cell debris and expose normally priveleged nuclear antigens (DNA, histones, nucleosomes, and Smith antigen (Sm)) to the immune system. This induces autoAb production, which in turn leads to the formation of IgG-immune complexes (IgG-ICs). When IgG-ICs are internalized by FcγRI they promote inflammatory responses. We find that defective lysosome acidification promotes the recycling of FcγRI/IgG-ICs within 48-72 hours of internalization. The recycled IgG-ICs are returned to the plasma membrane where they sustain FcγRI signaling, induce cytokine secretion, migration of cells to the kidney, B cell activation and they promote high levels of autoAb. These events contribute to end-organ disease.

The lysosome maturation defect and its consequences are evident in multiple models of spontaneous lupus, diabetes (NOD mice), and arthritis (K/BxN mice). It is also evident in approximately 67-75% of SLE patients experiencing active SLE. The defect is evident in multiple hematopoietic cell types including ex vivo Mφs, DCs, and B cells, and bone marrow-derived Mφs because of the cross-reactivity of FcγRI for C-Reactive Protein (CRP), an opsonin abundant in fetal bovine serum that coats apoptotic debris.

### Model

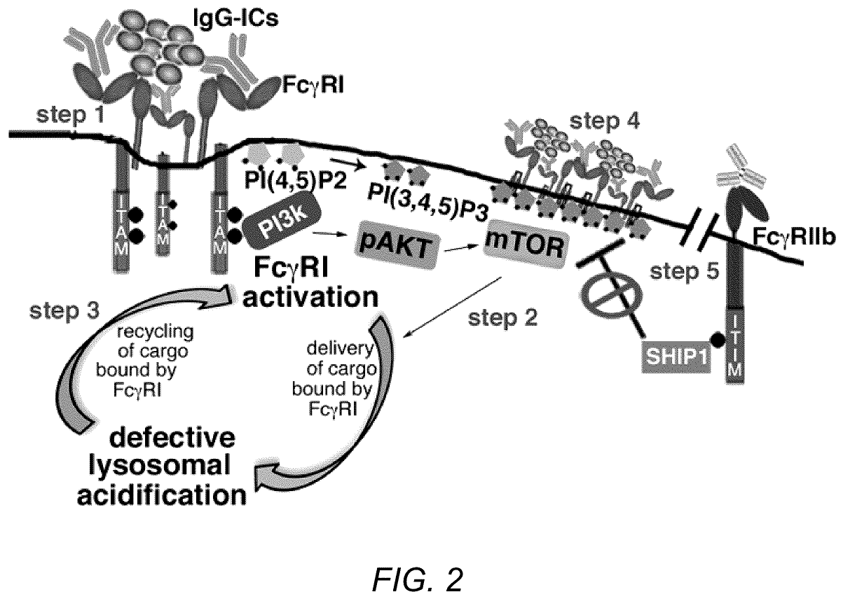

Early in the auto-immune response, a breakdown of B cell tolerance leads to extrafollicular autoAb. AutoAbs to nuclear antigens opsonize apoptotic debris forming IgG-ICs that crosslink FcγRI on myeloid cells, including Mφs (FIG. 2 step 1). Although Mφs from lupus-prone mice internalize IgG-ICs, they are not efficiently degraded due to defects in lysosomal maturation (decreased acidification, increased ROS; FIG. 2 step 2). Consequently, IgG-ICs/FcγRI recycle to the cell membrane (FIG. 2 step 3) where they accumulate (FIG. 2 step 4). Further, internalized IgG-ICs remain inside the cell 48-72 hours before they are recycled. This has several consequences. First, the prolonged time within the cell allows apoptotic debris to progress to secondary necrotic cell-derived material (SNEC), thus exposing autoantigens in an inflammatory context. Second, it prolongs exposure of endosomal TLRs to ligands. Third, it induces permeabilization of the phagolysosomal membrane. This allows IgG and nuclear antigens within the phagolysosome to leak into the cytosol. Undegraded dsDNA bind to AIM2 leading to pyroptosis. Leaked IgG activates TRIM21, which heightens IRF7 facilitating IFNαproduction when endosomal TLRs are concurrently activated by nucleic acids. Further, recycled receptors that accumulate on the surface of Mφs sustain signal transduction creating a feedforward loop wherein diminished lysosomal acidification and recycling of FcγR1-bound IgG-ICs result from, and lead to, chronic FcγRs signaling (FIG. 2, steps 2 and 3). Thus, chronic FcγRI signaling is not a primary defect; rather it is induced by the inability of the lysosome to degrade IgG-ICs. The feedforward loop is attenuated by either lowering the uptake of IgG-ICs (FcγRI^{-/-}/MRL/lpr), or decreasing ligand. This was revealed using the AID^{-/-}/MRL/lpr model which is unable to induce IgG class switch, and therefore does not make endogenous IgG ligand. Neither of these gene deletions (FcγRI or AID) change the underlying defect; rather, they reduce the disease provoking consequences. Thus, we have identified how immune complexes formed by apoptotic debris/SNEC and binding to FcγRI promote chronic immune activation in lupus. The data suggest that attenuating chronic FcγRI activation by disrupting the feedforward loop could be an effective lupus therapy because it is upstream of multiple pathologies.

### Other Evidence Implicating FcγRI in Disease

### FcγRs on Hematopoietic Cells Are Important in Lupus

Deposits of IgG-ICs have long been a focus in lupus nephritis. Two pieces of evidence suggest that renal deposits of immune complexes (ICs) are secondary to activating FcγRs on hematopoietic cells. First, bone marrow chimeras showed that expression of activating FcγRs (FcγRI, FcγRIII, FcγRIV) on hematopoietic cells, rather than kidney mesangial cells, was required for lupus nephritis. Second, under treatment conditions that diminished proteinuria and morbidity (BAFF neutralization), deposits of IgG and complement persist in the kidneys. Thus, renal deposits of IgG-ICs are evident in the absence of renal disease. Collectively, these findings suggested that FcγRs on hematopoietic cells (and not kidney mesangial cells) may initiate immune dysregulation leading to lupus nephritis, and that deposits of IgG-ICs in the kidney are not sufficient to induce renal disease. This supports the idea that immune dysregulation induced by IgG-ICs binding FcγRs on hematopoietic cells promotes renal disease, and changes the focus in SLE from how ICs contribute to end-organ disease to the upstream events leading to the ICs. What specific FcγRs were involved and how they promoted disease remained unclear.

### Conflicting Data About the Role of FcγRs in SLE

Elevated levels of IgG-ICs were first correlated with SLE in 1949. Because IgG-ICs were later found to bind FcγRs, their involvement in disease was assumed. Despite the strong correlation between IgG-ICs and disease, FcγRs have fallen from vogue as central players in SLE, although the evidence refuting their role is weak. The Fc receptor common gamma chain (FcRγ) is a receptor subunit encoded by FCER1G that is contained within FcγRI, FcγRIII, and FcγRIV, and important in FcγR-mediated signal transduction. Contradictory studies show that ablation FcRγ in MRL/lpr mice has no effect on disease progression, while loss of FcRγ in NZB/W F1 reduces disease. To further complicate these contradictory findings, it was later shown that loss of FcRγ did not completely ablate the expression of FcγRI, and that the remaining FcγRI was partially functional. Thus, the studies of activating FcγRs were at best inconclusive. Despite this, no one pursued a “clean” Fc common gamma chain knockout, or assessed the effects of individual FcγR deficiency on a lupus-prone background until our study of FcγRI^{-/-}/MRL/lpr.

### Loss of FcγRI in Lupus-prone MRL/lpr Mice is Sufficient to Restore Immune Regulation

We backcrossed B6/Fcγb RI^{-/-}mice (non-autoimmune-prone background) onto the MRL/lpr background (lupus-prone) generating FcγRI^{-/-}/MRL/lpr mice. We found that loss of FcγRI reduced the levels of nuclear antigen on the surface of hematopoietic cells from MRL/lpr mice consistent with a role for FcγRI in the recycling of undegraded SNEC/apoptotic debris. We also found decreased activation of signaling effectors downstream of FcγRI. Further implicating FcγRI in lupus, we found that autoAb, BAFF levels, and migration of hematopoietic cells to kidney were all markedly reduced, and the FcγRI^{-/-}/MRL/lpr mice did not develop lupus nephritis. Thus, loss of FcγRI is sufficient to attenuate disease indicating it is a key receptor in promoting lupus-related pathology. This finding was corroborated by assessing whether loss of IgG (in the presence of FcγRI) was also sufficient to attenuate disease. Using a passive transfer model wherein pathogenic anti-nucleosome is injected into AID^{-/-}/MRL/lpr mice (IgG-deficient), we found that IgG-ICs are necessary for B cell expansion, BAFF secretion, autoAb production, cell migration to kidney, and kidney disease. Thus, FcγRI and IgG are central players in disease, and defects in lysosome function may impact immune homeostasis because of the multitude of inflammatory consequences driven from chronic FcγR signal transduction.

### Lysosome pH Measurement

Absolute pH vs. Relative pH

The acidotropic dye LysoSensor™ Yellow/Blue can be used to quantify the pH or lysosomes using ratiometric imaging. We found that lysosomes from B6 Mφs (non-autoimmune) achieved an absolute pH of 4. In contrast, MRL/lpr Mφs fail to sustain a pH below 5.5. Absolute pH determination is ascertained relative to pH standards. To adapt this method to complex splenic cell populations and to analyze more cells than can be achieved by microscopy, we adapted this method to ratiometric flow cytometry. For this adaptation, we loaded cells with an acidotropic dye and concurrently analyzed cells by microscopy (with pH standards) and flow cytometry. We found that when B6 Mφs achieved a pH of 4, the “relative pH” obtained by ratiometric flow cytometry ranged from 0.6-0.7. When MRL/lpr Mφs achieved a pH of ˜5.5, this corresponded to a “relative pH” ranging from 0.85-0.95. Throughout this application, we use ratiometric flow cytometry to measure relative lysosomal pH of splenic cell populations from mice, and peripheral blood cells from SLE patients.

### Identification of FcγRI and FcγRIIb as Targets for Lupus Therapeutic

### Chronic PI3k Activity Impairs Lysosome Acidification in MRL/lpr Mφs

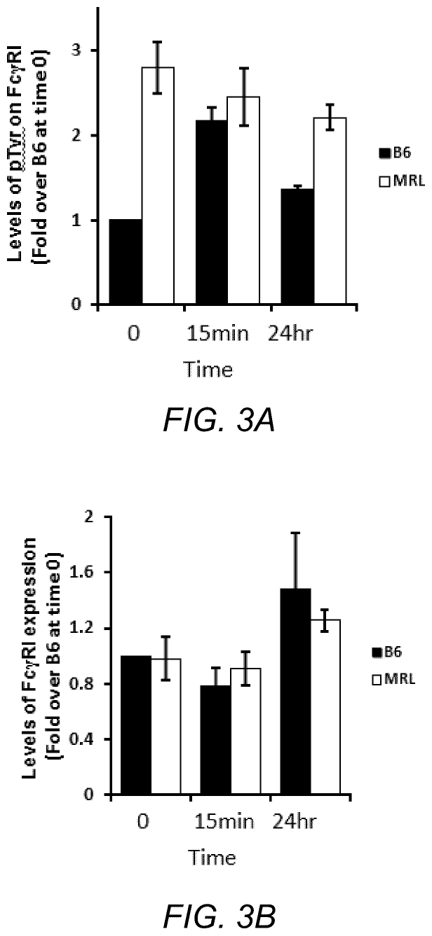

Chronic FcγRI signal transduction diminishes lysosome function and promotes recycling of IgG-ICs in MRL/lpr Mφs. Signaling through FcγRI occurs when the cytoplasmic ITAM motif is dually phosphorylated. To identify whether FcγRI was constitutively phosphorylated, we stimulated B6 and MRL/lpr Mφs over time, then immunoprecipitated FcγRI. We found that the basal levels of phosphotyrosine (pTyr) were elevated 3-fold in MRL/lpr Mφs compared to Mφs from B6 mice (FIG. 3A). Upon stimulation, the FcγRI pTyr levels increased ˜3-fold in B6 Mφs while the levels in MRL/lpr Mφs were unchanged. This was not due to varying amounts of FcγRI because the levels of total FcγRI protein were comparable (FIG. 3B).

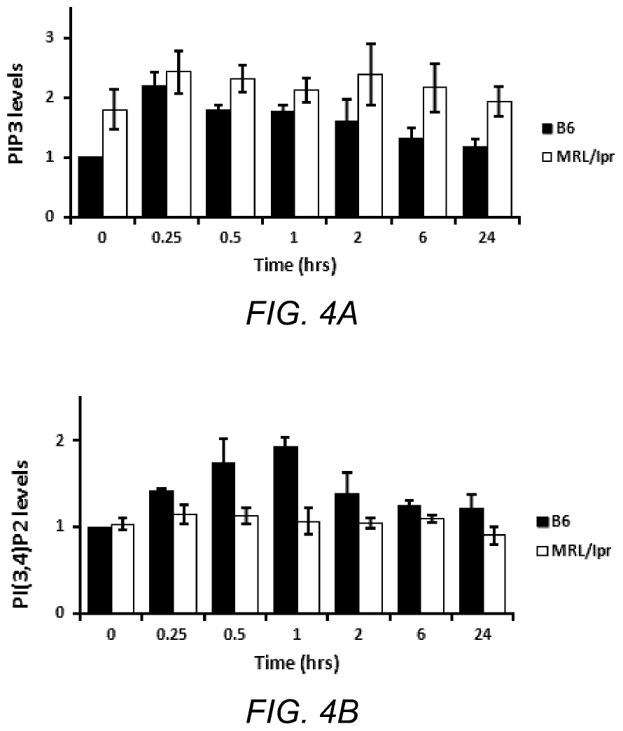

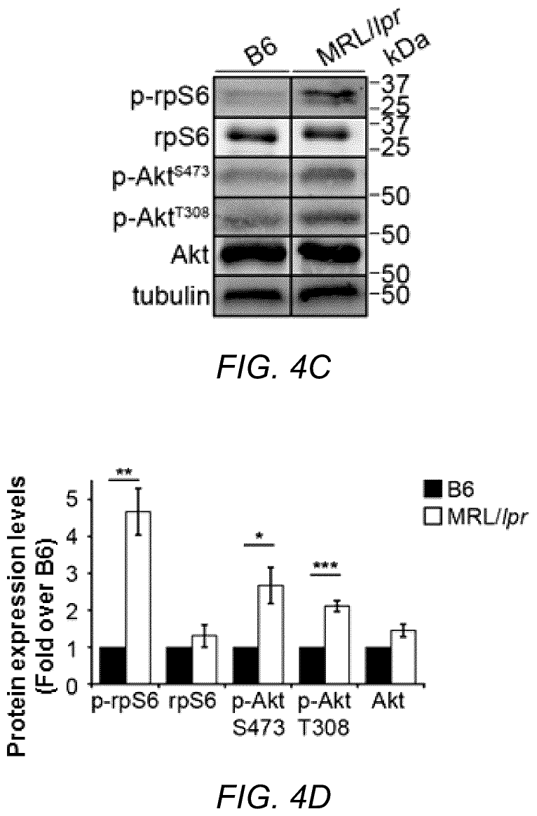

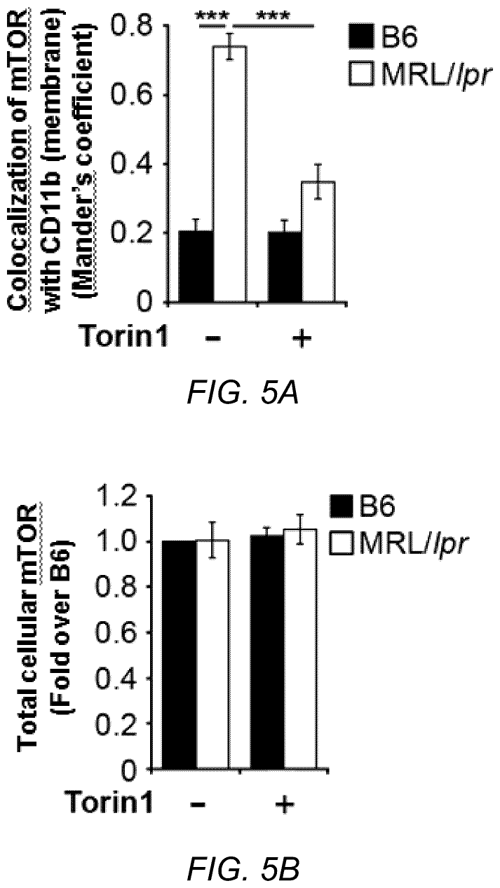

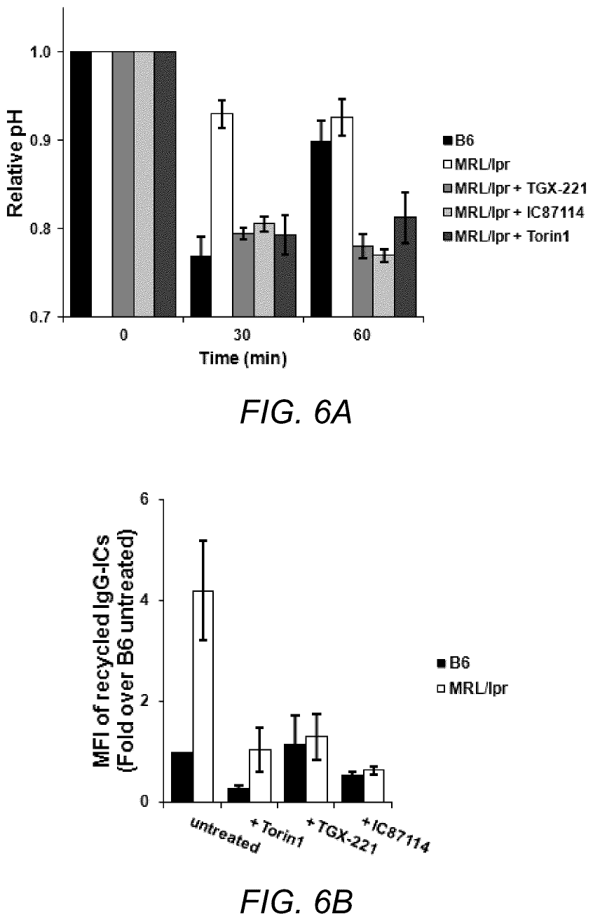

To understand why FcγRI signaling was not terminated in MRL/lpr Mφs, we studied the receptor proximal signaling events coupled to FcγRI signaling, namely the PI3k/Akt/mTOR pathway. PI(3, 4, 5)P₃(PIP₃) produced by PI3 kinase (PI3k) is critical in activating Akt and mTORC2. Consistent with increased PI3k activity, MRL/lpr Mφs have increased levels of PIP3 (FIG. 4A). SHIP is a 5′ inositol phosphatase that dephosphorylated PIP3 to produce PI(3, 4)P2. We find that while stimulated B6 Mφs show ˜2.4-fold increases in the levels of PI(3, 4)P2 the levels in MRL/lpr Mφs are only increased 1.3-fold (FIG. 4B). This indicates that the dephosphorylation of PIP3 is reduced in MRL/lpr Mφs. Concurrently, MRL/lpr Mφs have significantly elevated levels of phosphorylated Akt (pAkt) and ribosomal protein S6 (rpS6) (FIG. 4C, 4D) as would be expected when PI3k remains active and PIP3 levels remain high. Lastly, we find that mTOR is mislocalized in MRL/lpr Mφs, appearing at the plasma membrane rather than in the cytosol (FIG. 5A, 5B). To determine whether increased FcγRI signal transduction through the PI3k/Akt/mTOR pathway impaired lysosome maturation, we inhibited PI3k β(TGX-221), PI3kδ (IC87114) and mTORC1/C2 (Torin1) and quantified lysosome acidification following phagocytosis of IgG-ICs. We found that inhibiting p110β (TGX-221), p110δ (IC87114) or Torin1 restored lysosome acidification to levels comparable to B6, and the vesicles remained acidified for the duration of the treatment (FIG. 6A). Consistent with PI3k activity promoting the recycling of IgG-ICs, treating MRL/lpr Mφs with p110β and p110δ prevented the recycling of IgG-ICs (FIG. 6B). Further, treatment with Torin1 restored mTOR to a cytosolic location (FIG. 5A, 5B). These data indicate that chronic PI3k activity underlies heightened Akt and mTOR activity in MRL/lpr Mφs, and is upstream of lysosome acidification and the degradation of IgG-ICs. The defect that allows FcγRI to remain chronically active was unknown; however, it could be a potential target in restoring lysosome function and facilitating the clearance of IgG-ICs from the cell membrane.

### Diminished De-phosphorylation of PIP3 Implicates Defects in SHIP1

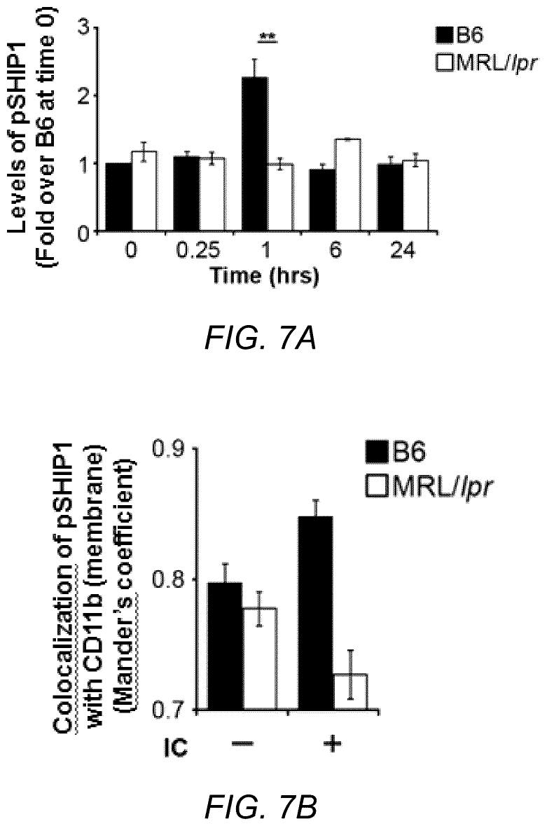

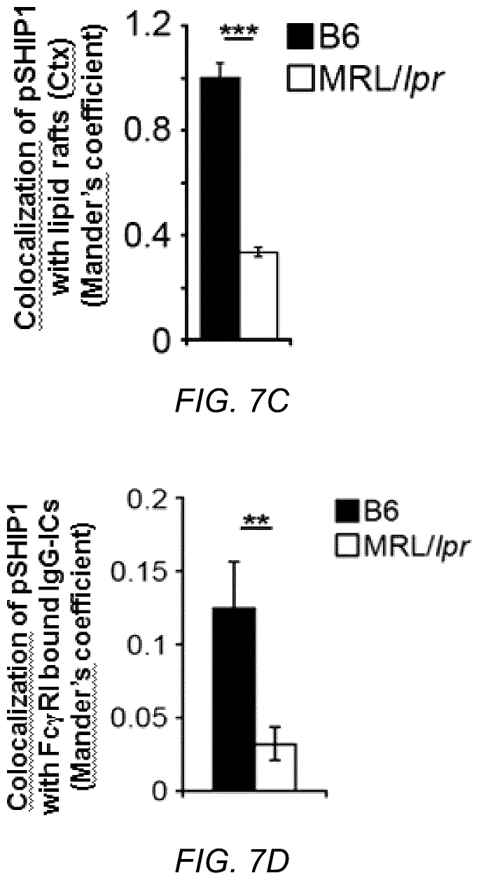

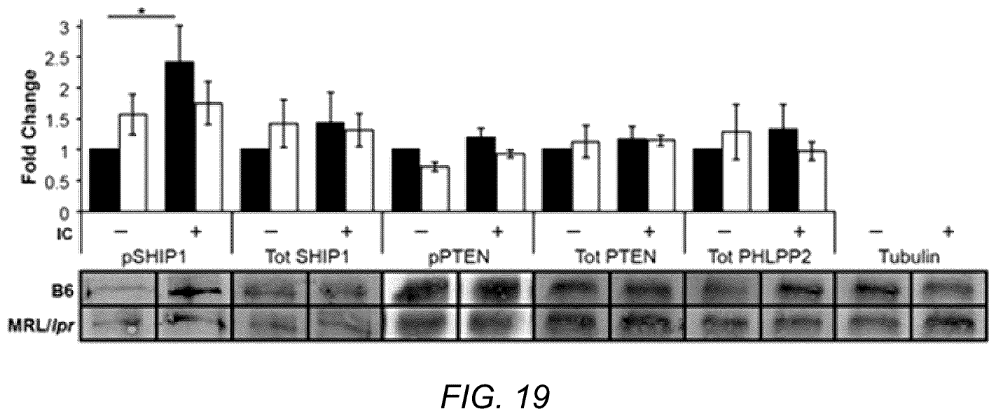

Multiple phosphatases are critical in regulating FcγRI signaling including: SHP-1 (de-phosphorylates the activating ITAM motif), SHIP1 and PTEN, (decreases PIP3 levels), and PHLPP2 (de-phosphorylates phospho-Akt^{S473}). Therefore, we assessed the activation of these signaling effectors in B6 and MRL/lpr Mφs prior to and following stimulation (1 hour) with IgG-ICs. We found that in stimulated B6 Mφs, the levels of pSHIP1 were increased 2.9-fold (FIG. 7A). In contrast, pSHIP1 levels were unchanged in stimulated MRL/lpr Mφs. Levels of phosphorylated protein or total SHP-1, PTEN, and PHLPP2 in MRL/lpr Mφs were not significantly different compared to B6 (FIG. 19). SHIP-1 dampens FcγRI activation (FIG. 2; step 5) through the de-phosphorylation of the 5′ phosphate of PI(3, 4, 5)P3 producing PI(3,4)P2. In B6 Mφs, the levels of PI(3, 4)P2 increase 2.4- and 2.1-fold at 0.5 and 1 hour following phagocytosis of IgG-ICs consistent with pSHIP1 activity (FIG. 4A). In contrast, the levels of PI(3, 4)P2 in MRL/lpr Mφs were only 1.3- and 1.2-fold increased (FIG. 4B). This is consistent with the idea that pSHIP1 is not de-phosphorylating PIP3 and suggests that MRL/lpr mice harbor a SHIP1 defect.

The phosphorylation and activity of SHIP1 depends on its association with membrane proteins including FcγRIIb, Dok1, and Lyn. Therefore, the impaired phosphorylation of SHIP1 in MRL/lpr Mφs could reflect an inability of SHIP1 to be recruited to the plasma membrane. Prior to stimulation, B6 and MRL/lpr Mφs have similar levels of pSHIP1 localized to the membrane (FIG. 7B). Following a 1 hr stimulation with IgG-ICs, B6 Mφs show a 1.6-fold increase in pSHIP1 localization to the membrane, in contrast to a 1.3-fold decrease in MRL/lpr Mφs. This suggests that when MRL/lpr Mφs are stimulated with IgG-ICs, SHIP-1 is not properly localized at the plasma membrane.

FcγRI constitutively resides within lipid rafts (25), while FcγRIIb requires receptor crosslinking to localize within lipid rafts. Since the activation of SHIP1 requires recruitment to FcγRIIb, the mislocalization of SHIP1 in MRL/lpr Mφs could reflect an inability of FcγRIIb to gain access to lipid rafts. The inability of MRL/lpr Mφs to recruit FcγRIIb into the lipid rafts coincides with 3-fold less pSHIP1 in cholera toxin (CTx) positive regions on the membrane (lipid rafts) when compared to B6 Mφs (FIG. 7C). To corroborate the findings that pSHIP1 fails to localize with FcγRI, we used TIRF microscopy to analyze colocalization of pSHIP1 with IgG-IC bound FcγRI. We found that pSHIP1 colocalized with FcγRI in B6 bone marrow derived macrophages (BMMφs); however, FcγRI and pSHIP1 showed a 3.9-fold decrease in colocalization in MRL/lpr BMMφs (FIG. 7D). This confirms that pSHIP1 is defective in localizing with FcγRI. Collectively these findings indicate that the phosphorylation and localization of pSHIP1 is impaired in MRL/lpr Mφs, and that pSHIP1 fails to localize in proximity to FcγRI within the lipid rafts. This in turn prevents the regulation of FcγRI signal transduction allowing PIP3 levels to remain high, pAkt to remain active, and increased mTOR activity to reduce lysosome function. Thus, the “off switch” that attenuates FcγR1 signal transduction is disabled in lupus-prone Mφs. This creates a feedforward loop wherein FcγRI loaded with IgG-ICs continue to arrive and chronically transduce signals at the plasma membrane. Our therapeutic proposes to attenuate the feedforward loop by crosslinking FcγRI with FcγRIIb to activate SHIP1 and promote co-localization with active FcγRI.

### Crosslinking FcγRI With FcγRIIb is Sufficient to Restore Lysosome Function

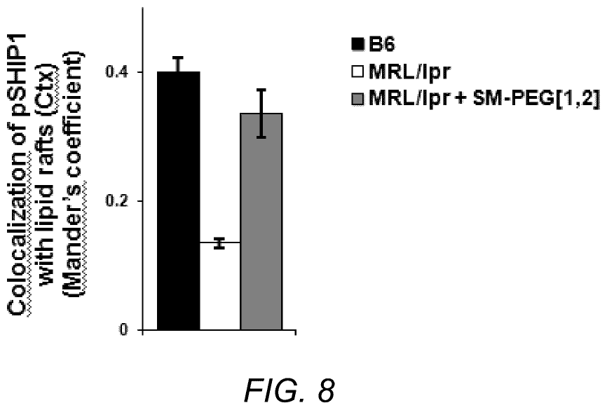

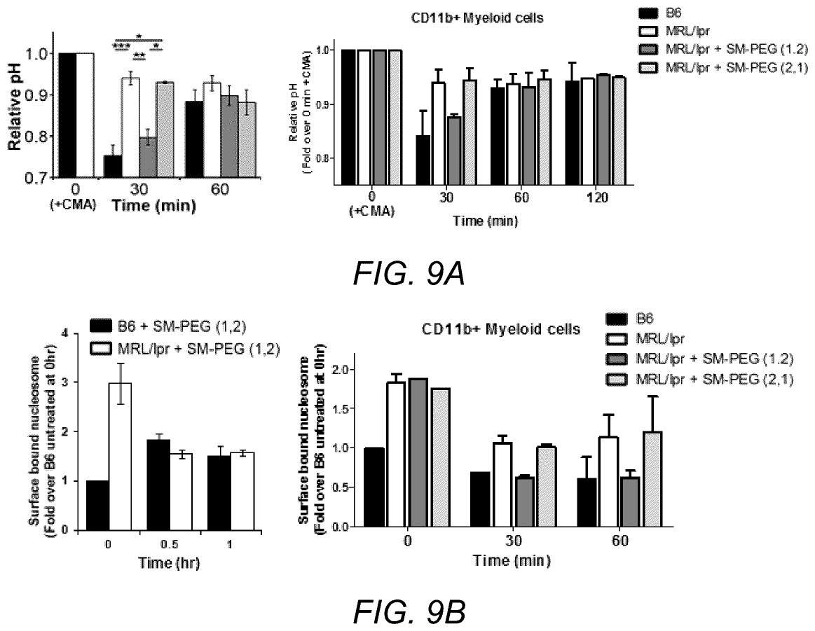

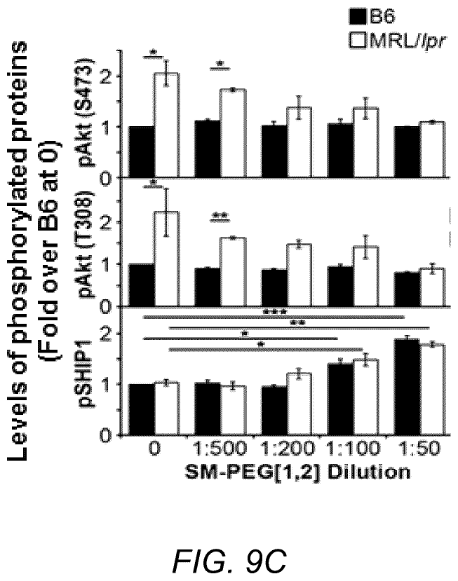

Identifying that the chronic activation of the PI3k/Akt/mTOR pathway is integral to lysosome dysfunction, and SHIP1 is impaired and mis-localized, raises the possibility that forcing FcγRIIb, the inhibitory receptor that recruits SHIP1 to dephosphorylate PIP3, into the proximity of FcγRI may attenuate the feedforward signaling loop, restore lysosome function, and clear the plasma membrane of recycled IgG-ICs. To assess this, we conjugated antibodies specific for murine FcγRI and FcγRIIb to a heterobifunctional PEGylated SMCC to induce receptor crosslinking. This molecule, SM-PEG[1 ,2], had a single anti-FcγRI surrounded by 3-4 anti-FcγRIIb. We also made a negative control PEG construct, SM-PEG[2, 1], where multiple anti-FcγRI antibodies co-ligated a single FcγRIIb. We found that treatment with SM-PEG[1, 2] restored the subcellular location of pSHIP1 to lipid rafts (CTx positive regions on the membrane) on MRL/lpr Mφs (FIG. 8). In addition, treatment with SM-PEG[1,2] reduced pAkt (S473/T308), and restores pSHIP1 to levels comparable to B6 (FIG. 9C). Thus, the crosslinking of FcγRI with FcγRIIb restored PI3k/Akt/mTOR signaling in MRL/lpr Mφs to levels comparable to B6.

Since chronic PI3k signaling is upstream of impaired lysosomal maturation (FIG. 6A), aggregating FcγRIIb with FcγRI should restore lysosomal acidification. Treating MRL/lpr Mφs with SM-PEG[1, 2] re-establishes lysosomal acidification in MRL/lpr Mφs to levels comparable to B6 (FIG. 9A). In contrast, treatment with SM-PEG[2, 1] has no effect. Coinciding with restored acidification, MRL/lpr BMMφs and ex vivo Mφs treated with SM-PEG[1, 2] showed a 2 and 2.5-fold decrease in accumulated IgG-ICs on the surface of the cell after 1 hour of treatment (FIG. 9B). Thus, crosslinking FcγRIIb with FcγRI is an effective strategy to restore lysosomal maturation and acidification, clear the plasma membrane of recycled IgG-ICs, to diminish PI3k/pAkt activation and restore pSHIP levels. These are key events disrupting immune homeostasis in SLE.

### Recycled IgG-ICs Appear on Lymphoid Cells, in Other Murine Models of SLE, and in Active SLE Patients

### Nuclear Antigens Accumulate on Other Hematopoietic Cells in Lupus-prone Mice

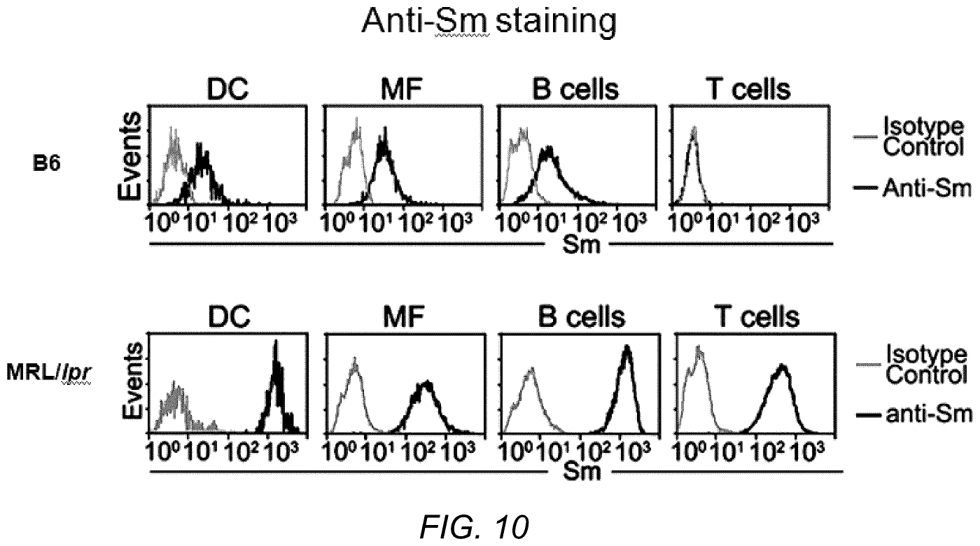

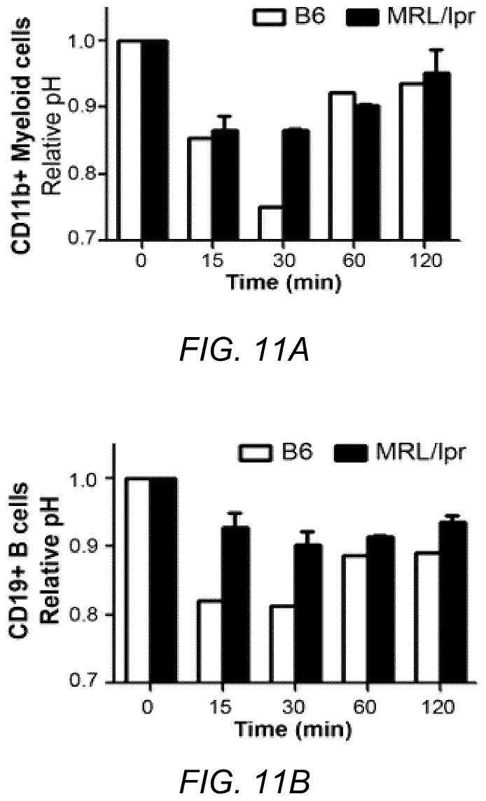

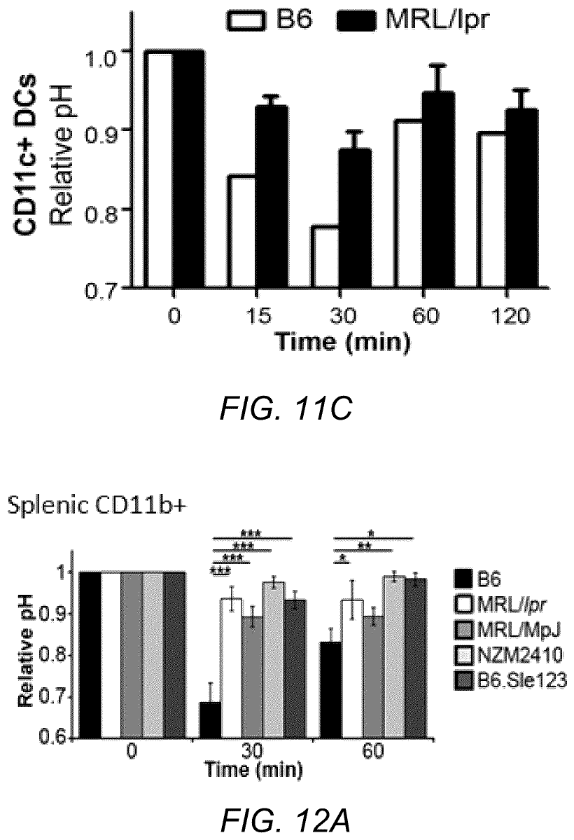

Our studies have focused on mechanisms underlying lysosome defects in macrophages because MRL/lpr mice lacking FcγRI (FcγRI^{-/-}/MRL/lpr) fail to develop disease. Since expression of FcγRI is limited to myeloid cells, yet lymphoid cells showed markedly reduced activation or cytokines secretion, or autoAb production, we concluded that defects in myeloid cell activation precede those of lymphoid cells. However, our data show that nuclear self-antigens such as DNA, Smith (Sm), and histones, accumulate on DCs, Mφs, and B cells, and these cells exhibit defects in lysosomal acidification (FIG. 10, FIGS. 11A-11C). This suggests that defects in lysosome function may occur across multiple cell lineages. Since B and T cells do not express surface FcγRI, the receptors displaying nuclear antigens on these cells must be unique relative to myeloid cell, raising the possibility that their unique properties may contribute to B and T cell defects in SLE.

### Defects in Lysosome Maturation are Evident in Genetically Unrelated Models of Murine Lupus

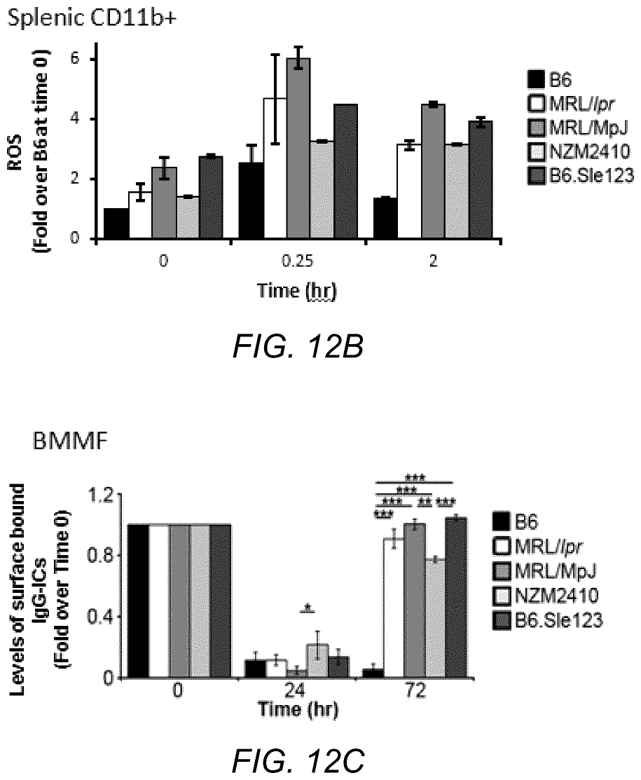

Our data characterizing the molecular and cellular basis for the lysosomal maturation were done using the MRL/lpr model. To assess whether the lysosome defect occurred in other models of SLE, we assessed [symbol] vivo CD11b+ splenocytes from the genetically unrelated NZM2410 and the Sle123 congenic mice. We found they also showed impaired lysosome acidification (FIG. 12A), prolonged and heightened ROS (FIG. 12B), and they recycled high levels of IgG-ICs (FIG. 12C) much like splenic Mφs from MRL/lpr mice. This shows that the lysosome defect is not unique to the MRL/lpr model. Since MRL/lpr and NZM2410 mice are genetically unrelated, our findings suggest that the lysosome defect may arise by different mechanisms/genetics, but confer a similar disease phenotype.

### The Consequences of Lysosome Defects May Be Relevant in Other Autoimmune Diseases

Lysosome defects may underlie other autoimmune diseases. We find that nuclear antigens and IgG accumulate on Mφs from mice prone to diabetes (NOD). Further, high levels of IgG are found on the surface of Mφs from mice prone to arthritis (K/BxN; however, the IgG found on Mφs from K/BxN mice is not bound by nuclear antigens. Whether diabetogenic antigens such as insulin, or arthritogenic antigens such as collagen or citrillinated proteins, form IgG-ICs that promote lysosome dysregulation is of interest.

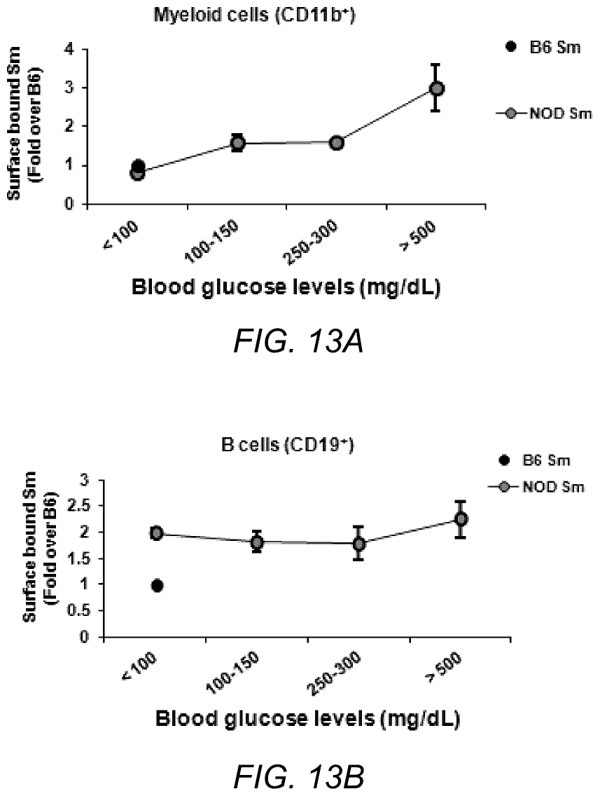

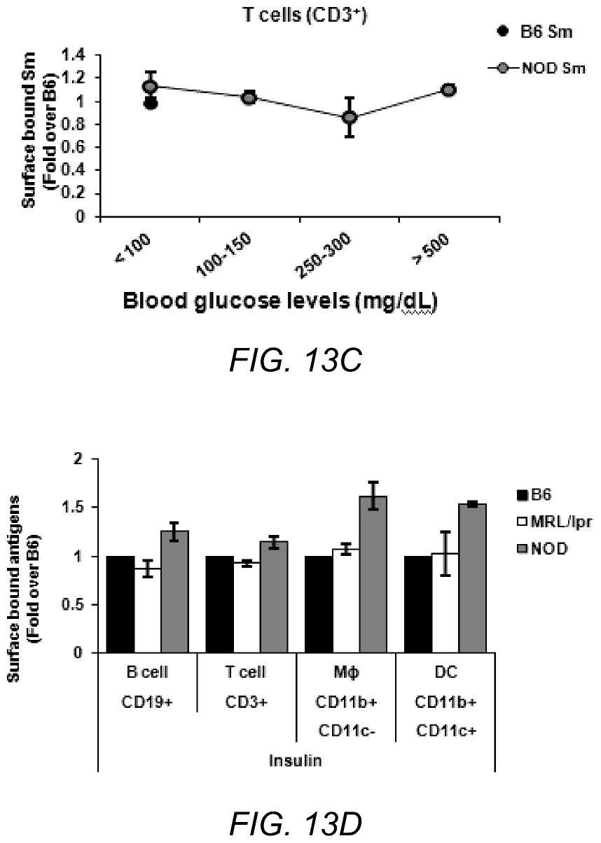

To further characterize NOD mice, we examined peripheral blood mononuclear cells (PBMCs) from mice with varying glucose levels (i.e., different stages of disease). In pre-diabetic mice (blood glucose<100 mg/dL), we found that nuclear antigens (Sm) on CD11b+myeloid were not present on the cell surface. However, in diabetic mice (blood glucose>500 mg/dL), accumulation of Sm on the surface of CD11b+myeloid cells was 3-fold higher than B6 controls (blood glucose<100 mg/dL) (FIG. 13A). On B cells from pre-diabetic (blood glucose <100 mg/dL) mice, surface bound Sm levels were increased 2-fold compared to B6 cells. Once the mice were fully diabetic (blood glucose>500 mg/dL) the accumulation of Sm on B cells increased to 2.5-fold greater than B6 (FIG. 13B). T cells showed no significant increase in nuclear antigen levels at all glucose levels compared to B6 mice (FIG. 13C).

To assess whether diabetogenic antigens accumulate on the surface of splenic B cells, T cells, Mφs, and DCs in pre-diabetic mice (blood glucose<100 mg/dL), we examined the levels of surface insulin. We found that insulin levels were modestly increased on the myeloid cells (1.5-1.6-fold) and B cells (1.3-fold), but not on T cells (1.1-fold) (FIG. 13D). Fully diabetic (blood glucose>500 mg/dL) NOD mice did not show accumulation of insulin (data not shown). The findings that nuclear antigens, as well as diabetogenic antigens, accumulate on hematopoietic cells during the course of murine diabetes raise the possibility that other autoimmune diseases harbor underlying defects in lysosome function. The heightened cell death in many autoimmune diseases could propagate inflammation by forming IgG-ICs with either nuclear antigens, or with disease-specific antigens.

### Defects in Lysosome Maturation Are Evident in SLE Patients Experiencing Active Disease

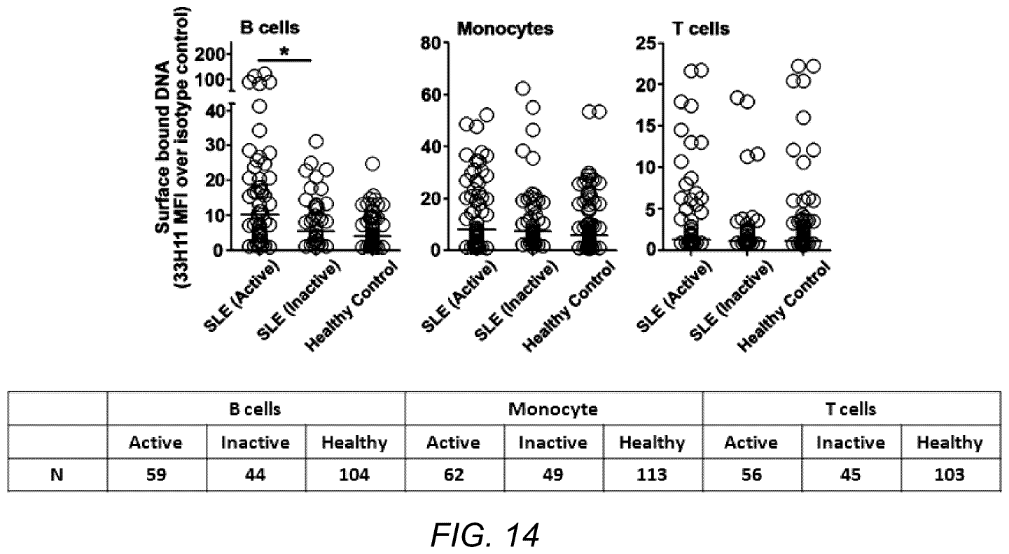

Our studies identify lysosome defects, heightened ROS, and the recycling and accumulation of IgG-ICs on multiple hematopoietic cell in genetically unrelated murine models of lupus. This raises the question of whether this murine defect is evident in human SLE. If so, does it associate with active or inactive disease. To assess relevance in human disease, we initiated a cross-sectional study of active (SLEDAI≥6), inactive (SLEDAI<6) SLE patients, and healthy controls. We found levels of DNA (Sm, and nucleosome) were elevated at least 2-fold on B cells of approximately 75% of SLE patients experiencing active SLE (FIG. 14). The levels of DNA and IgG on monocytes and T cells were not statistically different. We were perplexed why the levels of nuclear antigen were not elevated on monocytes since in mice, splenic Mφs showed levels of surface nuclear antigen that were 84-fold compared to B6. One possibility was simply that murine and human lupus were mechanistically unique. A second possibility was that human peripheral blood monocytes and murine tissue Mφs were different. To sort this out, we isolated peripheral blood monocytes and splenic Mφs from mice and assessed whether blood and tissue cells from the same animal showed differing results. Indeed, blood monocytes from B6 and MRL/lpr mice failed to show any differences in Sm or nucleosome, while tissue Mφs from the MRL/lpr mice showed higher levels of Sm, nucleosome, and DNA compared to Mφs from B6 mice. This suggests that either monocytes encounter less nuclear antigens in blood; or alternatively, monocytes that have encountered antigen rapidly migrate to the tissue.

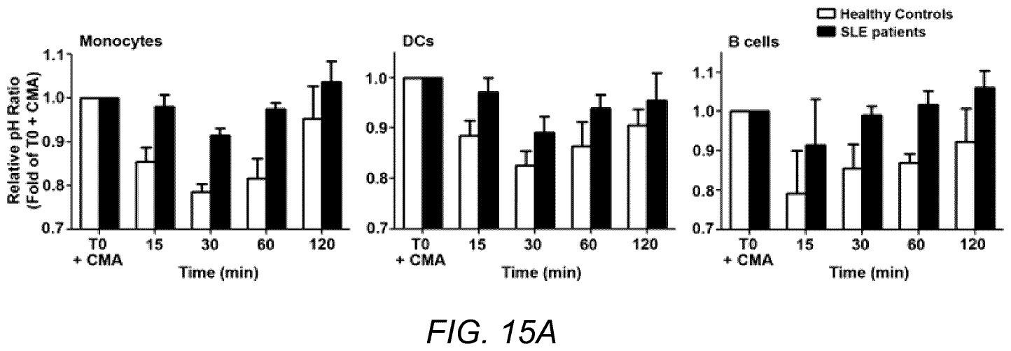

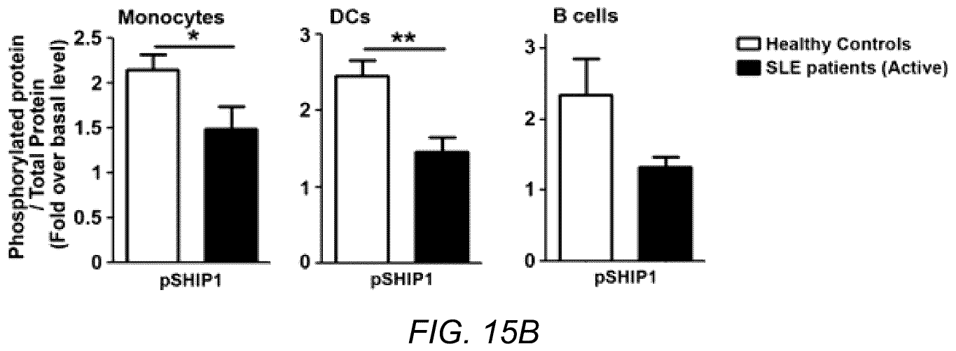

To assess whether lysosome acidification was defective in human peripheral blood monocytes (Mo), dendritic cells (DCs), and B cells, we monitored stimulated peripheral blood cells with IgG-ICs and then measured lysosome acidification over time using ratiometric flow cytometry. Monocytes from healthy controls (HC) acidified within 30 minutes of stimulation showing a 22% reduction in relative pH. In contrast, relative lysosome pH in monocytes from SLE patients was only reduced 9% (FIG. 15A). Thus, although blood monocytes in many SLE patients did not show increased antigen levels relative to HC, their lysosomes harbor an underlying defect, that when challenged by IgG-ICs, reduces lysosome function. Lysosome function in DCs and B cells was also reduced, but to a lesser extent than monocytes (FIG. 15A). It has been previously reported that the ability of murine DCs and B cells to acidify is reduced compared to Mφs, possibly as a mechanism that optimizes antigen processing for presentation of peptides to T cells.

To assess whether effectors in the PI3k pathway were elevated in blood monocytes, DCs, and B cells, we measured the levels of phospho-SHIP1 (pSHIP1) in peripheral blood monocytes from active and inactive SLE patients (FIG. 15B). We found that in the presence of stimulation HC Mo, DCs and B cells increased SHIP1 phosphorylation 2-2.5 fold compared to unstimulated cells. In contrast, SLE patients increased phosphorylation of SHIP1 by only 1.2-1.5 fold. This indicates that like murine SLE, human disease also exhibits defects in SHIP1 phosphorylation.

### Summary

To summarize, we identified a defect in lysosome maturation defined by diminished acidification and heightened ROS, recycling of IgG-ICs bound to FcγRs, and accumulation of nuclear antigens and IgG on the cell surface. The defect is evident in MRL/lpr, NZM2410, NOD, K/BxN, and B6/Sle123. It is also evident in approximately 75% of SLE patients experiencing active disease. Defects are evident in multiple hematopoietic cell types, including ex vivo Mφs, DCs, and B cells. It is also evident in BMMφs likely because of the cross-reactivity of FcγRI for C-Reactive Protein (CRP). Diminished lysosome function is not a consequence of Fas, or simply due to an increased burden of apoptotic cells, because Mφs from MRL/MpJ show diminished lysosome function, but those from B6/lpr or B6/MerTK^{kd} mice do not. Our data support a therapeutic designed to correct lysosomal acidification by attenuating FcγRI signaling via coligation with FcγRIIb. This data is supported by our findings that pSHIP1 (associated with FcγRIIb) is not present in lipid rafts where FcγRI resides, a finding corroborated by TIRF microscopy showing that pSHIP1 is not localized near FcγRI. We also show that cross-linking FcγRI with FcγRIIb is sufficient to clear the plasma membrane of recycled receptors, attenuate FcγRI/PI3k activation, restore SHIP1 phosphorylation and localization to lipid raft, and restore lysosome acidification.

### Design of a Murine Therapy

### Rationale

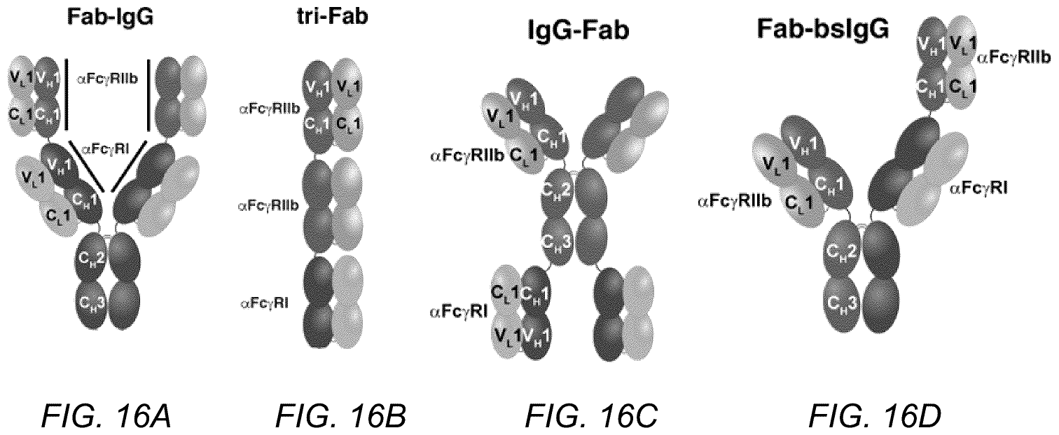

Our data identify that FcγRI is chronically active in murine lupus, in part due to the inactivity of SHIP1. Although SHIP1 is associated with several ITIM-containing receptors, we found that crosslinking FcγRIIb with FcγRI is effective in dampening FcγRI activation, restoring lysosome function, and decreasing the recycling and accumulation of surface FcγRs bound by IgG-ICs. Thus, our therapeutic will crosslink these two targets using bispecific antibodies of either mono- or multi-valency. We have designed several antibody-based molecules to achieve crosslinking of FcγRI with FcγRIIb. Many unusual antibody structures can be engineered. Four structures to achieve co-ligation are shown in FIGS. 16A-16C. In addition to bi-specificity, our design will use mutations that enable two Fab moieties to be assembled simultaneously with 95% correct heavy (H) chain/light (L) chain pairing simply by co-expression. This platform maintains the C_{H}1 and C_{L} IgG architecture thereby minimizing aggregation and increasing protein stability and solubility, and it allows for multi-valency. One design is a monovalent, bispecific molecule where one FcγRI crosslinks one FcγRIIb. However, if multivalency is necessary to achieve adequate repression of FcγRI activation, we designed multivalent bispecific antibodies capable of crosslinking multiple FcγRIIb receptors to FcγRI. Unique conformations include, but are not limited to, molecules containing two anti-FcγRIIb ratioed to two anti-FcγRI, three anti-FcγRIIb to one anti-FcγRI, or two anti-FcγRII to one anti-FcγRI. The molecules arrange the Fab regions uniquely on an Fc-containing IgG backbone. However, they can also be arranged as tandem Fabs without a complete Fc region (for example, see tri-Fab in FIG. 16B). Our basic goal is bispecificity. If an increased number of FcγRIIb/SHIP1 molecules are needed to repress FcγRI, we will increase the valency by engineering more complex bsAbs where we can recruit multiple FcγRIIb molecules/FcγRI molecule. Further, many antibodies that target FcγRI and FcγRIIb will work in crosslinking these targets.

### Multivalent Bispecific Antibodies Functions Comparably to SM-PEG[1, 2 ]

Although the SM-PEG[1, 2] molecule allowed us to create high valency, the PEG-based molecule is incompatible with a sustained treatment regiment need to treat lupus. As such, we generated four multivalent, bispecific antibody constructs whose protein products crosslink FcγRI with FcγRIIb to assess whether multivalency in a bi-specific antibody format functioned comparably to the SM-PEG[1, 2] (FIGS. 16A-16C). Two constructs had a 2:1 valency. One was a trivalent Fab (tri-Fab) lacking an Fc region (FIG. 16B). The other construct had an appended Fab on one heavy chain of a bispecific IgG (Fab-bsIgG) (FIG. 16D). Two constructs had a 2:2 valency. Both were tetravalent and contained an intact IgG. One had Fab domains appended to the N-terminus of each heavy chain (Fab-IgG; FIG. 16A). The other construct (IgG-Fab; FIG. 16C) had Fab domains appended to the C-terminus of each heavy chain. All constructs encode the same antibodies as SM-PEG[1, 2].

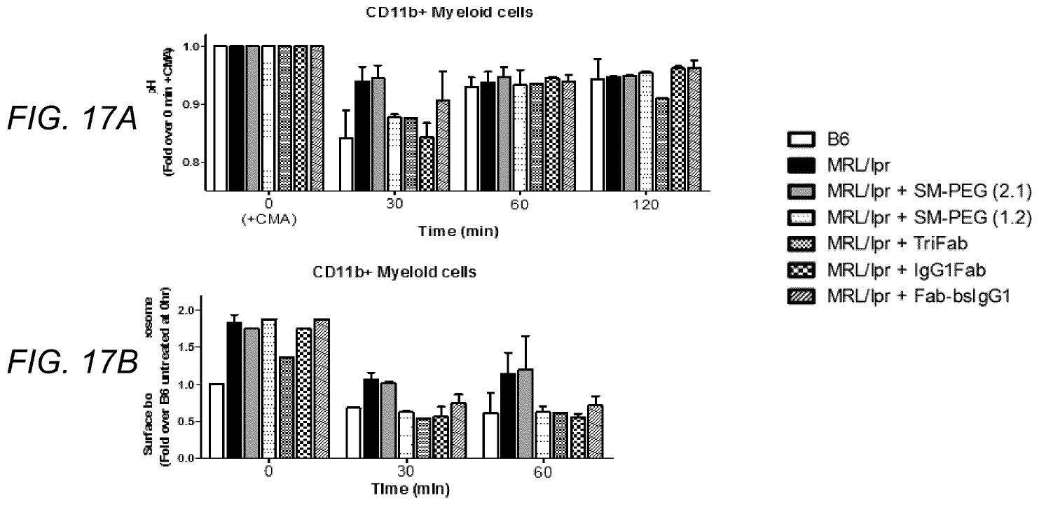

Three of the four constructs (Tri-Fab, IgG-Fab, and Fab-bsIgG), expressed well and these bound FcγRI and FcγRIIb comparable to hybridoma antibodies (data not shown). We tested their crosslinking capacity relative to SM-PEG[1, 2] and control PEG (SM-PEG[2, 1]). We found that Tri-Fab and IgG-Fab restored lysosome acidification comparably to SM-PEG[1, 2] while Fab-bsIgG was slightly less efficient (FIG. 17A). We also found that all 3 constructs cleared IgG-ICs from the plasma membrane as well as SM-PEG[1, 2] (FIG. 17B).

### Design of a Human Therapy

As extensively discussed above, our goal in treating mice is to crosslink activating FcγRI with inhibitory FcγRIIb. However, in humans, two activating FcγRs have been identified, FcγRI and FcγRIIa. Like mice, humans have one inhibitory FcγR, FcγRIIb. Thus, our design for a human therapeutic is to make two bispecific antibody constructs. The first will crosslink human FcγRI with human FcγRIIb. The second construct, we will crosslink human FcγRIIa with human FcγRIIb. These constructs could be used separately or in combination depending on which activating FcγR is implicating in promoting inflammation in human SLE. In summary, our human targets are: FcγRI, FcγRIIa, and FcγRIIb.

In terms of the bispecific design for our human therapeutic, our basic goal is bispecificity. If an increased number of FcγRIIb/SHIP1 molecules are needed to repress FcγRI and/or FcγRIIa, we will increase the valency by engineering more complex bsAbs where we can recruit multiple FcγRIIb molecules/FcγRI and/or FcγRIIa molecule. Further, many antibodies that target FcγRI, FcγRIIa, and FcγRIIb will work in crosslinking these targets. Thus, the antibodies in this therapeutic must bind human FcγRI, FcγRIIa, and FcγRIIb.

### Methods

### Animals

C57BL/6 (B6) and MRL/MpJ-Tnfrs6^{lpr}/J (MRL/lpr; JAX stock number000485) colonies were maintained in an accredited animal facility at the University of North Carolina. NZM2410 mice were from Medical University of South Carolina and B6.Sle123 mice were from University of Florida. MRL/MpJ mice were purchased from JAX (stock number 000486).

Human samples

Patients with SLEDAI scores≥6, as defined by the Systemic Lupus Erythematosus Disease Activity Index (SLEDAI), were selected for enrollment after informed consent in accordance with the University of North Carolina or Duke University institutional internal review board. Peripheral blood samples were collected with sodium heparin (BD Biosciences) during routine clinic visits.

### Cells

BMMφ Cultures were prepared from single-cell suspensions of bone marrow were prepared from the tibias and femurs of mice. Mononuclear cells were isolated by using Lympholyte Separation Medium (CEDARLANE Laboratories), plated in a 60-mm Petri dish with 6 mL of MF differentiation media [D10 media with 10% (vol/vol) L-cell supernatant], and cultured overnight (37° C., 5% CO). Nonadherent cells were plated into nontissue culture-treated 100-mm Petri dishes (0.75-1 mL cells per Petri dish) with 7 mL of fresh MF differentiation media. To promote MF differentiation, cells were incubated for 6 d (37° C., 5% CO2) with an additional 5 mL of MF differentiation media being added on day 4. The resulting BMMFs were removed from the dish by washing with ice cold PBS. BMMF cultures were 98% CD11b⁺, I-A^{1o}, and B7.2^{1o}+.

Mouse splenocytes and human PBMCs were prepared into single cell suspension in PBS after RBC lysis (0.165M NH₄Cl, pH 7.2 or ACK buffer), then processed differently for each assay. In some assays, splenic DCs or Mφs were isolated using MicroBeads for CD11c+ (DCs) or CD11b+ (Mφs) following manufacturer's instructions (Miltenyi Biotec).

### Formation of Immune Complexes

IgG-ICs were created as previously described (Monteith et al., 2016). Briefly, thymocytes from B6 mice (6-8 weeks old) were irradiated (600Gys) and incubated in PBS for overnight at 37° C. Next day, supernatant was collected by pelleting irradiated thymocytes and ICs were formed by adding anti-nucleosome IgG2a (6.67 μg/1 ml supernatant). Following the incubation on ice for 30 min, IgG-ICs were pelleted by centrifugation at 40,000 rpm for 45 min. Pelleted IgG-ICs were resuspended in R10 media (10% FBS in RPMI).

### Cell Stimulation and Treatments

Cells (0.25×10⁶) were rested in R10 media for 1 hr at 37° C. prior to the addition of any reagents. IgG-ICs were added, incubated for given duration, and removed at each time points. For some assays, cells were treated with TGX-221 (p110β), IC87114 (p110δ), or Torin1 (mTorC1/C2) 2 hours prior to the addition of IgG-ICs and the reagents were remained in the cell suspension throughout the duration of the experiments. For testing therapeutic antibodies and SM-PEG(1, 2), IgG-ICs were added to rested cells simultaneously with SM-PEG(1, 2) (5 μg/ml), Tri-fab (40 μg/ml), IgG-Fab (40 μg/ml), Fab-bsIgG (40 μg/ml), or control SM-PEG(2, 1) (5 μg/ml), and incubated for indicated time points.

### Flow Cytometry

To analyze surface bound antigens, cells were blocked in 2.4G2 for 30 min at 4° C. in FACs media (PBS with 2% FBS, 0.02% NaN₃), washed, stained with biotinylated or fluorochrome conjugated anti-Sm (2.12.3), anti-nucleosome (PL2-3), anti-DNA (33H11), anti-insulin, and/or anti-IgG for 30 min at 4° C., washed, stained for cell makers (CD11b for Mφs, CD14 for monocytes, CD11c for DCs, CD19 for B cells, CD3 for T cells) and/or streptavidin-Alexa Fluor® 647 in FACS media for 30 min at 4° C. and washed. Cells were fixed in 2% paraformaldehyde in FACS media and incubated for 15 minutes at 4° C. Cells were resuspended in FACS media and the MFI of the surface bound antigens and IgG were determined by flow cytometry and normalized to an isotype control staining. For measuring ROS production, dihydrorhodamine 123 was added 20 min prior to fixation. Cells were resuspended in FACs media and the MFI of the indicated proteins were determined by flow cytometry and normalized to an isotype control. For intracellular staining, cells were stained for cells makers as above and fixed in 4% paraformaldehyde in FACS media. Fixed cells were stained with antibodies to AKT, pAKT^{ser}, pAKT^{thr}, S6, pS6, SHIP1, pSHIP1 in perm buffer (FACS media with 0.05% Saponin) for 30 min on ice, washed in perm buffer, then stained with goat-anti-rabbit IgG-Alexa Fluor® 647 in permeabilization buffer for 30 min on ice. Washed cells were resuspended in FACS media and the MFI of the indicated proteins were determined by flow cytometry and normalized to an isotype control. The expression levels of intracellular kinases were calculated as follows: (MFI of phosphorylated signaling molecule/MFI of isotype control)/(MFI of total signaling molecule/MFI of isotype control). Samples were acquired on an 18-color LSR II Flow cytometer and FACSDiva 8.0.1 software (Becton Dickinson) or Cyan flow cytometer and Summit software (Beckman Coulter).

### Ratiometric Flow Cytometry

Cells were incubated (37° C., 5% CO₂) for 2 hours prior to the addition of 40 μL of IgG-ICs in R10 media (as above). As an internal control wherein lysosomal acidification was inhibited, Concanamycin A (20 ng/mL) was introduced to one sample from each cell type 2 hours prior to addition of IgG-ICs and left on the cells throughout the experiment. Cells were stimulated with IgG-ICs for 30 min, 60 min, and 120 min at 37° C. At each time points, LysoSensor™ (2 mg/mL) was added and incubated for 15 min, then, analyzed by flow cytometry. The dye was excited with a UV laser (355 nm). Relative pH was quantified by ratioing the MFIs from the emission channels (450/20 nm, 585/42 nm) (normalized to concanamycin A control). All flow cytometry was conducted using an 18-color Becton Dickinson LSR II Flow cytometer, and data were acquired using Becton Dickinson FACSDiva 8.0.1 software.

### Recycling of IgG-IC Flow Cytometry

BMMφs were incubated (37° C., 5% CO₂) with 40 μL Alexa Fluor® 488-labeled IgG-ICs in R10 media (as above). To quantify surface bound ICs at 0 hours, phagocytic uptake was impaired by culturing with IgG-ICs on ice for 2 hours. This was sufficient to allow the ICs to bind to the surface of the cell but not be phagocytosed. For all other time points, cells were incubated (37° C., 5% CO₂) for 2 hours, then media was replaced to remove all unbound ICs. At indicated time points, cells were blocked in 2.4G2 for 30 min on ice in FACS media, washed, and split into 2 samples. One sample was incubated with an anti-Alexa Fluor® 488 antibody (quenches Alexa Fluor® 488 fluorescence), while the other sample was left in FACS media for 30 min on ice. Both samples were washed and fixed with 2% paraformaldehyde and incubated at 4° C. for 15 min. Surface bound IgG-ICs were calculated by subtracting the MFI of the quenched sample (intracellular IgG-ICs) from the MFI of the unquenched sample (total IgG-ICs). Values were normalized to B6 0 hr.

### Fluorescence Microscopy

BMMφs were cultured with IgG-ICs in R10 media at 37° C. For measuring lipid rafts fluorescently labeled CTx was added to the culture 30 min prior to fixation. For the nuclear stain, hoechst was added 15 min prior to fixation. Mφs were fixed with 2% paraformaldehyde, then incubated at 4° C. for 15 min. Cells were blocked in 2.4G2 for 30 min at 4° C. in FACs media (PBS with 2% FBS, 0.02% NaN₃), washed, and stained with primary and corresponding secondary antibodies in permeabilization buffer (PBS with 0.05% Saponin and 0.5% BSA) for 30 min at 4° C. Mφs were imaged in FACs media or resuspended in FluorSave™ and imaged. All confocal microscopy was conducted using a Zeiss 710 confocal microscope with a 63×1.4 NA (oil) PLAN APO lens and Zeiss Zen software or Olympus 4-line TIRF microscope with a 63×1.4 NA (oil) PLAN APO lens and MetaMorph® software. Data were analyzed using ImageJ. Colocalization was measured by calculating the Mander's coefficient (colocalized pixels/total fluorescent pixels) in all the cells imaged.

### Immunoprecipitation

Lysates for immunoprecipitation were prepared by the addition of lysis buffer containing 1% CHAPS, 150 mM NaCl, 10 mM tris(hydroxymethyl)aminomethane (Tris™) (pH 7.5), 2 mM sodium orthovanadate, 1 mM PMSF, 0.4 mM EDTA, 10 mM NaF, and 1 μg/ml each of aprotinin, leupeptin, and α1-antitrypsin to cell pellets. Lysates were held on ice for 10 min followed by the removal of particulate material by centrifugation at 12,000×g for 10 min at 4° C.

Antibodies used in the immunoprecipitations were conjugated to cyanogen bromide-activated Sepharose™ 4B per the manufacturer's instruction (Amersham Pharmacia Biotech). Approximately 2 μg of precipitating antibodies was incubated with 1.5×10⁶ cell equivalents of cleared lysate for 2 hour at 4° C. Immunoprecipitates were washed twice with lysis buffer, and then resuspended in reducing SDS-PAGE sample buffer.

### Western Blot

Whole cells lysates or immunoprecipitates were fractionated by 10% SDS-PAGE. Separated proteins were transferred to Immobilon®-FL membranes, blocked in Li-cor Blocking Buffer, and then incubated with the various immunoblotting Abs followed by the appropriate fluorophore-conjugated secondary Abs. Immunoreactive proteins were detected using a LI-COR Odyssey® infrared imaging system with Odyssey® 3.0 software.

### Example 2

We recently reported that high levels of IgG-ICs accumulate on the surface of human and murine hematopoietic cells in SLE. On murine myeloid cells this is a consequence of diminished lysosomal acidification that prevents degradation of FcγR-bound IgG-ICs, promoting their recycling and accumulation on the cell surface. This leads to chronic signaling of FcγR that heightens BAFF and autoantibody levels, leads to autoreactive B cell expansion, and permeabilizes the phagosomal membrane allowing IgG and nuclear antigens to leak into the cytosol subsequently inducing IFNα production. Consistent with the idea that FcγRI is integral to the early events in SLE, loss of FcγRI in MRL/lpr mice reduces 95% of autoantibody and 90% of BAFF. Further, these mice do not develop renal disease.

Our preliminary data show that diminished lysosomal acidification is induced by events upstream of mTOR, linking chronic PI3k activation to diminished degradation of IgG-ICs. We also show that SHIP1 is integral in this defect, and that forcibly crosslinking FcγRI with FcγRIIb (recruits SHIP1) on murine macrophages restores lysosomal acidification, and diminishes activation of PI3k signaling. Our central hypothesis states that in active SLE patients, FcγRIIb fails to associate with FcγRI; thus, preventing SHIP1 activity and sustaining PIP3 levels. This promotes active disease by sustaining PI3k/mTOR signaling and diminishing lysosomal acidification creating a feed forward loop wherein diminished lysosomal acidification and recycling of FcγR1-bound IgG-ICs results from, and also leads to, chronic FcγRs signal transduction. Forcibly crosslinking FcγRI and/or FcγRIIa with FcγRIIb on monocytes should restore their ability to degrade incoming cargo by localizing SHIP1 near activating FcγRs, reducing PI3k-induced PIP3, and thereby regulating mTOR activity. In cross sectional and longitudinal studies, we will define whether defective lysosomal acidification correlates with active SLE, and involves defective lysosomal acidification and activation of the PI3k/mTOR pathway. We will also assess whether crosslinking human FcγR1 and/or FcγRIIa with FcγRIIb on peripheral blood monocytes diminishes PI3k signaling and restores lysosomal acidification.

### Specific Aims

We identified a cellular defect that lies upstream of several cellular and molecular events associated with systemic lupus erythematosus (SLE). It stems from the inability of lysosomes to degrade phagocytosed cell debris bound by autoAb (henceforth referred to as IgG-ICs). Diminished lysosomal degradation induces IgG-ICs that were phagocytosed by FcγRI and/or FcγRIV to recycle back to the plasma membrane. This leads to their accumulation, allowing ligand-bound FcγRI to chronically transduce signals. Chronic FcγRI signaling induces BAFF secretion, migration of cells to the kidney, and is upstream of B cell activation and autoAb. Further, the prolonged intracellular residency of IgG-ICs in the phagocytic system promotes TLR7/9 activation, and permeabilizes the phagolysosomal membrane. This allows nucleic acid and IgG to be released into the cytosol and activate innate sensors leading to heightened IFNα and pyroptotic cell death. This further exacerbates the apoptotic burden.

Our preliminary data identify that defective lysosomal maturation in MRL/lpr macrophages (MFs) is part of a vicious cycle wherein chronic FcγRI signaling and PI3k/mTOR activation diminish lysosomal maturation forcing undegraded FcγRI/IgG-ICs to recycle and accumulate on the cell surface allowing for continued activation of FcγRI. We show that forcibly crosslinking FcγRI with FcγRIIb using a multivalent, bifunctional antibody-based molecule restores lysosomal acidification, and diminishes the activation of the PI3k pathway. We hypothesize that during active disease, defective lysosomal acidification promotes the accumulation surface-bound IgG-ICs in human SLE, sustaining FcγR signaling, activating mTOR, and diminishing lysosome acidification (Aim 1b). Forcibly crosslinking FcγRI/FcγRIIa with FcγRIIb on human monocytes or FcγRIIa/FcγRIIb on B cells should restore their ability to degrade incoming cargo because it will localize SHIP1 near FcγRI or FcγRIIa reducing PI3k-induced PIP3 and thereby regulating mTOR activation and restoring lysosomal function.

### (a) Significance

Background: The paradigm in SLE has been that autoreactive B cells, having overcome tolerance, form germinal centers (GCs) where they produce isotype switched, IgG autoantibodies (autoAbs). The binding of IgG autoAbs to apoptotic debris forms immune complexes (henceforth referred to as IgG-ICs) that subsequently deposit in the kidneys of lupus patients, promoting lupus nephritis. In this model, B cells become the central effector due to their ability to make affinity matured IgG following GC responses; however, this model is more of an assumption rather than a proof of concept. Later studies showed that B cell responses to antigen can occur outside the GC, and these extrafollicular responses induce class switched IgG antibody. Further, loss of B cell anergy promotes extrafollicular B cell responses and short-lived plasma cells. In addition, the early formation of IgG-ICs and the cross-linking of FcγRs are required to induce robust GCs and memory responses. This supports the concept that early extrafollicular antibody responses play important roles in initiating long-term autoimmunity, but certainly does not rule out that a self-propagating amplification loop involving germinal center-induced IgG might sustain inflammation and contribute to disease. Nonetheless, rather than being the byproduct of disease, IgG-ICs could participate in the early inciting events of SLE, before germinal center involvement.

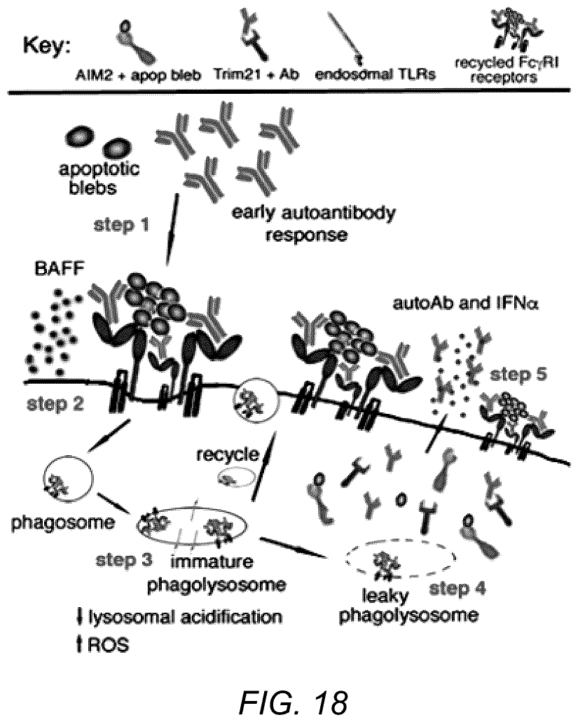

Chronic FcγR1 signaling induces defective lysosomal maturation, and promotes lupus-associated pathology: We identified a previously unappreciated defect in lysosome maturation in lupus-prone mice that affects SLE disease pathology. The defect is not unique to murine disease because flaring SLE patients show evidence of the defect. A model of our murine findings is summarized in FIG. 18. We find high levels of nuclear antigen (Sm, DNA, nucleosomes, histones) accumulate on the surface of murine dendritic cells (DCs), macrophages (MF), B and T cells (Step 1). In MRL/lpr mice, the accumulation of nuclear antigens requires the MRL/MpJ genetic background, and is not due to heightened levels of extracellular apoptotic debris, as B6/lpr and B6/MerTK mice do not accumulate surface nuclear antigen. This suggests that the accumulation of nuclear antigens on the cell surface may be related to the genetic background of the MRL line. Further, accumulation of surface nuclear antigens is apparent in NZM2410 mice, suggesting that the defect is not unique to MRL/lpr mice. Characterization of the nuclear antigens on myeloid cells shows they are immune complexes (ICs) binding FcγRI and FcγRIV. The surface accumulation is not due to diminished internalization, as internalization rates and intracellular trafficking patterns of gold-tagged IgG-ICs do not differ between B6 and MRL/lpr MFs. However, MFs from MRL/lpr mice show diminished lysosomal acidification, sustained ROS production, and within 72 hours of internalization, the IgG-ICs recycle back to the cell membrane (Step 2). This indicates that MRL/lpr MFs harbor a defect in lysosomal maturation that diminishes acidification and the ability to degrade IgG-ICs. As a consequence, FcγR-bound ICs recycle and accumulate on the cell surface. Diminished acidification is sufficient to induce recycling of IgG-ICs as B6 MFs treated with concanamycin A (inhibitor of lysosomal acidification by disrupting the vacuolar H+−ATPase) promotes the recycling of IgG-ICs. Thus, the accumulation of nuclear antigens on myeloid cells is not the consequence of failed clearance or aberrant intracellular trafficking; instead, it represents un-degraded IgG-ICs constitutively bound to FcγRs that have recycled to the cell membrane because of impaired lysosomal maturation and diminished acidification.

The failure to degrade IgG-ICs contributes to the pathology of SLE in MRL/lpr mice. First, diminished lysosomal degradation promotes the permeabilization of the phagolysosomal membrane (Step 3). This allows nucleic acid to escape into the cytosol activating AIM2, thereby promoting the formation of inflammasomes and the activation of caspase 1. This is consistent with ongoing pyroptosis that provides a renewed source of nuclear antigen for continued formation of IgG-ICs. Second, IgG that leaks into the cytosol binds TRIM21 inducing NFκB nuclear translocation and heightening the levels of cytoplasmic IRF7, an essential substrate for TLR7- and TLR9-induced IFNα secretion. Furthermore, the prolonged intracellular residency of nucleic acids in the phagolysosomal compartment activates TLR7/9, promoting the nuclear translocation of IRF7 and inducing IFNα secretion and additional autoantibody (Step 4). A third consequence of the accumulation of FcγR-bound IgG-ICs is heightened BAFF secretion. Heightened BAFF is evident in human SLE, and a therapeutic target in treating disease. In MRL/lpr mice, heightened BAFF is dependent on FcγRI and IgG because FcγRI^{-/-}/MRL/lpr mice do not exhibit high BAFF levels. Further, MRL/lpr mice that lack IgG (AID^{-/-}/MRL/lpr) do not exhibit high BAFF levels or disease, unless IgG autoantibody (anti-nucleosome IgG) is passively transferred.

Our goal is to (1) translate our murine findings to human disease, and (2) test a potential therapeutic designed based on molecular events identified in mouse and supported by human data. These studies are significant because:
(1) The idea that a cell biology defect underlies SLE is unexpected. Defining whether the murine findings are relevant in human disease is an important strategy to justify additional murine studies. Based on our pilot study, and the preliminary human findings below, the proposed studies could identify a lysosomal maturation defect in human disease.
(2) Our preliminary data indicate that defective lysosome maturation is induced by chronic mTOR activation, raising the possibility it underlies/contributes to active disease. If the cross sectional and longitudinal studies show that accumulation of nuclear antigens strongly correlates with active disease, and that it occurs coincident with heightened signaling through the PI3k pathway, SHIP1 mislocalization, and diminished lysosomal acidification, this would be significant because it provides the translational evidence necessary to justify additional murine studies aimed at identifying the events upstream of mTOR that disrupt PI3k signaling.
(3) New therapies in SLE are needed; especially rationale therapies based on defined molecular events that could target multiple pathways. The proposed studies will assess a therapeutic strategy wherein crosslinking FcγRI and FcγRIIa with FcγRIIb on human cells attenuates the vicious cycle of FcγR signaling, chronic innate receptor activation, diminished lysosomal degradation, and recycling of IgG-IC-bound FcγRs; events known to promote heightened cell death, BAFF, IFNα, autoreactive B cell expansion and autoantibody production, and lupus nephritis. If the human studies show that targeting the FcγR feed forward loop is efficacious, as found in mice, it directs future studies toward testing whether a murine FcγR crosslinking biologic in MRL/lpr mice can alter the course of disease.

### (b) Innovation

### Conceptual Innovation (New Ideas to Understand Disease)

SLE is a multigenic disease influenced by environmental factors. The standard of care includes anti-rheumatic drugs and immunosuppressive therapy. Recent target-specific therapies mostly support patients who are non-responsive to standard of care. The complexity of SLE makes it difficult to establish a single gene, molecular event, or cell type that “causes” lupus. Perhaps this explains why inactivating or depleting a single hematopoietic cell type, or neutralizing single cytokines has only modest effects in clinical trials. Therefore, therapies aimed at targeting defects upstream of multiple SLE pathologies may prove to be more efficacious in the clinic. Our model identifies that chronic IgG-ICs/FcγRI interactions and defective phagolysosomal maturation are upstream of BAFF, IFNα, pyroptosis/enhanced cell death, autoantibody production, and lupus nephritis indicating that targeting this defect could be a good therapeutic in SLE. This study is conceptually innovative because it proposes to define whether diminished lysosomal maturation identified in murine lupus is relevant in human disease.

### Technical Innovation (New Technology to Solve Complex Problems)

This application is technically innovative because we propose to crosslink FcγRI or FcγRIIa with FcγRIIb. For our initial in vitro study we will use a PEG molecule conjugated with the anti-FcγR antibodies. If forcibly crosslinking FcγRI or FcγRIIa with FcγRIIb on human monocytes restores lysosomal acidification and diminishes activation of the PI3k/mTOR pathway (see FIGS. 7C, 7D 9A, 9C), we will re-design IgG1 to create a multivalent, bispecific molecule with engineered interfaces that promote accurate heavy/light chain pairing. The technology allows us to assemble the variable domains of both heavy and light chains (V_{H}, V_{L}), encoding specificity for two FcγRI molecules. The multivalent design will aggregate FcγRI or FcγRIIa with FcγRIIb.

### (c) Approach

### (c1) Preliminary Data

What underlies the inability of MRL/lpr MFs to degrade IgG-ICs? Phosphoinositide 3-kinases (PI3k) play an important role in a variety of cellular functions, but only the p110β and p110δ subunits of PI3k have been demonstrated to play a role in FcγR-mediated phagocytosis. Upon receptor ligation, PIP3 from PI3k activation acts as a docking site at the membrane to recruit and activate Akt and mTORC2, leading to the downstream activation of mTORC1. To define whether the activation of the PI3k/Akt/mTOR pathway was linked to defective lysosomal maturation, we inhibited the activity of PI3k p110β (TGX-221), p110γ (IC87114), and mTORC1/C2 (Torin1). We found that MRL/lpr bone marrow macrophages

(BMMFs) pre-treated with TGX-221, IC87114, and Torin1 restored lysosomal acidification (FIG. 6A), while drugs that inhibit p110α and mTORC1 alone did not have an effect (data not shown). This indicates that heightened PI3k/Akt/mTOR activity is upstream of lysosomal maturation. To define whether signal transduction from FcγRI was dysregulated, we measured the levels of phosphorylated signaling effectors in the PI3k pathway. We found that the levels of phosphorylated S6 (pS6) or Akt (S473, T308) (pAkt) were elevated in unstimulated MRL/lpr when compared to B6 MFs (FIG. 4C, 4D). The level of kinase activation in MRL/lpr MFs were not further elevated upon stimulation with IgG-ICs. The levels of pPTEN and pPHLPP2 were comparable to B6, regardless of whether they were stimulated (FIG. 19). Strikingly, the levels of SHIP1 phosphorylation were increased in unstimulated MRL/lpr MFs, and did not further increase following IgG-IC stimulation. This was in contrast to the 2.3-fold increase in B6 MFs following stimulation with IgG-ICs (FIG. 19). The inability of IgG-ICs to induce pSHIP1 in MRL/lpr MF suggests it may show reduced activity, and therefore may be ineffective in regulating PI3k-induced PIP3 levels. Although regulators of the PI3k pathway were not identified in autoimmune GWAS, a number of reports identify defects in regulating PI3k in human SLE, and effectors in the PI3k pathway are important in B cell anergy.

Membrane localization is a crucial event in SHIP1 phosphatase activity. Using confocal microscopy, we found that pSHIP1 was present at the plasma membrane, and within the cytosol of unstimulated B6 and MRL/lpr BMMFs (data not shown). Stimulation of B6 BMMFs with IgG-ICs translocated pSHIP1 exclusively to the membrane and into lipid rafts (FIGS. 7B and 7C). In contrast, the translocation of pSHIP1 to the membrane lipid rafts in stimulated MRL/lpr BMMFs was 3-fold lower. Thus, pSHIP1 may be defective in localizing with FcγRI in the lipid rafts, which could impair its ability to regulate PIP3.

To define whether pSHIP1 localizes with FcγR1 we undertook total internal reflection fluorescence (TIRF) microscopy. This method allows fluorophore tagged proteins to be visualized within 200 nm of the glass-water interface, providing the resolution to assess the dynamics of receptors on, and recruitment of signaling effectors to, the plasma membrane. We found that B6 MFs stimulated with IgG-ICs efficiently colocalized pSHIP1 with FcγRI (FIG. 7D). In contrast, MRL/lpr MFs stimulated with IgG-ICs failed to colocalize pSHIP1 with FcγRI. Thus, pSHIP1 is not effectively recruited to FcγRI, consistent with heightened mTOR activity.

Since ITAM-containing FcγRs are regulated by activated SHIP1 through membrane colocalization via FcγRIIb, we generated a molecule that forcibly co-ligates FcγRI with FcγRIIb. We chose FcγRI since our previous studies showed that FcγRI^{-/-}/MRL/lpr mice show markedly reduced SLE phenotypes (BAFF, B cell expansion, autoantibody), and do not develop disease. The rationale for crosslinking FcγRs was that forcibly inducing the association of FcγRI with FcγRIIb would bring pSHIP1 to lipid rafts containing activated FcγRI, reduce overall PI3k/Akt/mTOR activation, and consequently restore lysosomal acidification. The molecule used a heterobifunctional PEGylated SMCC crosslinker that linked antibodies specific for FcγRI (X54-5/7.1) and FcγRIIb (2.4G2) via primary amines (NH2), or sulfhydryl (SH) groups. Anti-FcγRI was separated from anti-FcγRIIB by spacer arms containing 7 ethylene glycol units. Two molecules were created, one had a single anti-FcγRI surrounded by multiple anti-FcγRIIb (called SM-PEG[1, 2]), while the control molecule had a single anti-FcγRIIb surrounded by multiple anti-FcγRI (called SM-PEG[2, 1]). We found that treatment with SM-PEG[1, 2] restored acidification in MRL/lpr MFs while the control, SM-PEG[2, 1], had no effect of acidification (FIG. 9A left panel). Further, treatment with SM-PEG[1, 2] restored pAkt (S473/T308) and pSHIP1 to levels comparable to B6 (FIG. 9C). These results suggest that forcibly aggregating FcγRIIb with FcγRI may be an effective strategy to restore lysosomal maturation and prevent downstream SLE phenotypes.

Our studies further advance the data summarized in FIG. 18 by showing that FcγRs are chronically signaling, thereby promoting defective lysosomal maturation (FIG. 18, step 1) through activation of the PI3k pathway. This reveals a feedback loop wherein diminished lysosomal acidification and recycling of FcγR1-bound IgG-ICs (FIG. 18, step 2) results from, and also leads to, chronic FcγRs signal transduction (FIG. 18, step 3). Participation of FcγRI in a vicious cycle supports our findings that MRL/lpr mice deficient in FcγRI (FcγRI^{-/-}/MRL/lpr) do not develop disease. Our data implicate FcγRIIb and pSHIP1 in enabling this defect through their diminished localization with FcγRI (FIG. 7D and FIG. 18; step 4) and diminished pSHIP1 phosphorylation (FIG. 19). This application proposes to translate our murine data to assess whether lysosome maturation is defective in human SLE perhaps contributing to heightened cell death, BAFF, IFNα, autoreactive B cell expansion, autoantibody production, and lupus nephritis as identified in mice. Importantly, since the molecular events underlying this defect are becoming clear, lysosome defects may be targetable in a therapeutic that could alleviate multiple lupus-related pathologies (Aim 3).

We hypothesize that in MRL/lpr MFs, FcγRIIb fails to associate with FcγRI; thus, preventing SHIP1 activity and sustaining PIP3 levels. We propose this promotes active disease in human SLE patients by sustaining PI3k/mTOR signaling, and diminishing lysosomal acidification. This creates a vicious loop wherein diminished lysosomal acidification and recycling of FcγR1-bound IgG-ICs results from, and also leads to, chronic FcγRs signal transduction through sustained PI3k/mTOR. We expect that forcibly crosslinking FcγRI and/or FcγRIIa with FcγRIIb on monocytes or B cells will restore their ability to degrade incoming cargo by localizing SHIP1 near FcγRI or FcγRIIa reducing PI3k-induced PIP3 and thereby regulating mTOR activity.

### (c2) Experimental Strategy

### Aim 1

In a cross-sectional study, define whether human hematopoietic cells accumulate surface IgG-IC during flare, and whether accumulation of surface IgG-ICs on human cells coincides with the signaling events promoting diminished lysosome acidification.

### Rationale and Hypothesis

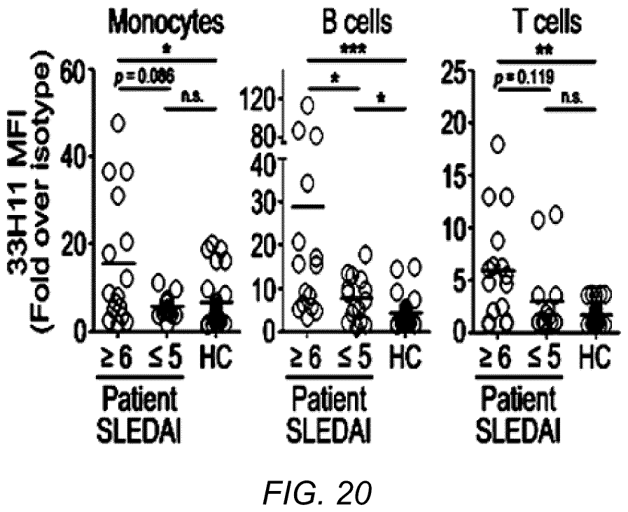

Diminished lysosomal acidification in MRL/lpr MFs is dependent on high activation of mTOR and/or the sustained mis-localization of active mTOR at the cell membrane. Pharmacologically inhibiting mTOR in MRL/lpr MFs corrects the membrane mis-localization, allows lysosome acidification, and degradation of IgG-ICs. Thus, diminished lysosomal acidification is downstream of mTOR and a consequence of FcγRI activation that leads to the recycling of FcγR-bound IgG-ICs and the accumulation nuclear antigens on murine hematopoietic cells. In this aim, we propose a cross sectional study to identify whether accumulation of IgG and nuclear antigens is evident in SLE, whether it is unique to active SLE disease (as compared to another autoantibody positive autoimmune disease, seropositive RA), and whether patients exhibiting heightened levels of surface nuclear antigen show concurrent activation of the PI3k/mTOR pathway and diminished lysosomal function. Our preliminary data show that 67-75% of SLE patients experiencing active disease (SLEDAI≥6) display a greater than 2-fold increase in the levels of surface nuclear antigens (FIG. 20) on peripheral blood mononuclear cells (PBMCs). We hypothesize that the inability to efficiently degrade IgG-ICs is not unique to mice but will also be evident in human disease. As a consequence, IgG and nuclear antigens will accumulate on the cell surface and promote chronic FcγR signaling and perpetuate defects in lysosomal acidification. Our goal is to correlate kinase activity with the accumulation of nuclear antigens on DCs, MFs, B and T cells. Thus, the activation state of effectors within the PI3k/mTOR pathway will be monitored each time the patient is assessed for levels of surface ICs.

### Aim 1a

Quantify the level of IgG and nuclear antigens (IgG-IC) on peripheral blood monocytes, B and T cells from active SLE (SLEDAI≥6), inactive SLE (SLEDAI≤5) and RA patients.

### Experimental Design

We will also use flow cytometry to determine whether IgG and DNA accumulate on the cell surface by quantifying surface IgG, DNA (33H11) and nucleosomes (PL2-3) on peripheral blood monocytes (CD14+), B-cells (CD19+) and T-cells (CD3+) from active SLE patients, healthy controls, and RA control patients as shown in FIG. 20. The specificity of 33H11 and PL2-3 were validated using an autoantigen array containing 95 autoantigens, including nuclear and non-nuclear antigens (UTSW Microarray Core).

### Aim 1b

Define whether patients who accumulate nuclear antigen and IgG exhibit activated PI3k/mTOR, diminished lysosomal acidification, and whether exogenously loaded IgG-ICs recycle.

### Experimental Design

(i.) We will assess whether the accumulated ICs coincide with elevated PI3k/mTOR signaling, we will use flow cytometry and intracellular staining to quantifying the levels of phosphorylated S6, Syk, and Akt (pS6, pAkt ^{S473}/pAkt ^{T308}) in B-cells (CD19), monocytes (CD14,) and T-cells (CD3). This method is established. We will also assess the location of mTOR (FIG. 5A, 5B) and pSHIP1 (FIG. 7B) in monocytes from active compared to inactive SLE and HC using confocal microscopy. Our murine data show these signaling effectors are mis-localized in MRL/lpr but not B6 MFs.

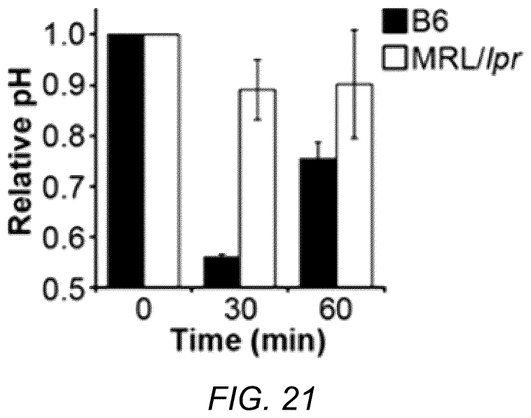

(ii.) We will assess whether human monocyte from the 3 groups show diminished lysosomal acidification. We have established that we can use peripheral blood to assess acidification of monocytes (FIG. 21) and B cells (data not shown). We find that monocytes from B6 (FIG. 21) and healthy donor peripheral blood (FIG. 15A) acidify within at ˜30 minutes, while monocytes from MRL/lpr peripheral blood fail to acidify, results that parallel splenic macrophages. To measure vesicular pH by flow, we will stimulate peripheral blood mononuclear cells (PBMCs) with pre-formed IgG-ICs. These are prepared by binding anti-nucleosome (PL2-3) to apoptotic blebs (18 hr irradiated human thymoma). Using ratiometric flow we will quantify the pH of the vesicles surrounding the ICs (FIG. 9A left panel, and FIG. 21). For positive and negative controls, we will use HC PBMCs treated with concanamycin A. Untreated HC PBMCs degrade IgG-ICs, while those treated with concanamycin A have impaired lysososomal acidification (due to pharmacological inhibition of the lysosomal V-ATPase). In addition, we will coculture a subset of cells with Torin 1 (250 ng/ml) to assess whether mTOR plays a role in the acidification of human MFs.

(iii.) To assess whether IgG-ICs recycle in patients from the 3 groups, we will use Mo-MFs derived as described above. Again, the Mo-MFs will be loaded with pre-formed IgG-ICs except the anti-nucleosome will be labeled with Alexa Fluor® 488. At varying time points (0, 48, 72, 96 hrs), samples will be fixed, then split into two, and one quenched with anti-Alexa Fluor® 488. Samples will be analyzed by flow cytometry. The levels of external ICs can be calculated by the difference between the unquenched sample and the anti-Alexa Fluor® 488-quenched sample. Our preliminary data indicate that HC monocytes from peripheral blood acidify the lysosomal compartment upon stimulation with IgG-ICs at a timepoint comparable to splenic MFs indicating that measuring relative pH can be applied to human PBMCs (FIG. 15A).

### Aim 2

In a longitudinal study, define whether the accumulation of IgG-ICs on human hematopoietic cells waxes and wanes with SLE relapse and remission, and whether this correlates with PI3k/mTOR activation, mislocalization of pSHIP1, diminished lysosomal acidification, and serum IFNα and BAFF levels.

### Rationale and Hypothesis

Our findings that IgG and nuclear self-antigens accumulate in SLE patients experiencing active disease raise the possibility these events might wax and wane as disease flares and remits. This is important because it might indicate that accumulation of nuclear antigens contributes to active disease in a subset of SLE. A longitudinal study where disease remission serves as an internal control for each patient will allow us to assess whether disease activity correlates with the burden of surface IgG-ICs. We hypothesize that as SLE relapses and remits, the levels of surface IgG-ICs rise and fall, coincident with activation of PI3k/mTOR and diminished lysosomal acidification. We also predict that BAFF and IFNα levels correlate with surface accumulation of nuclear antigens.

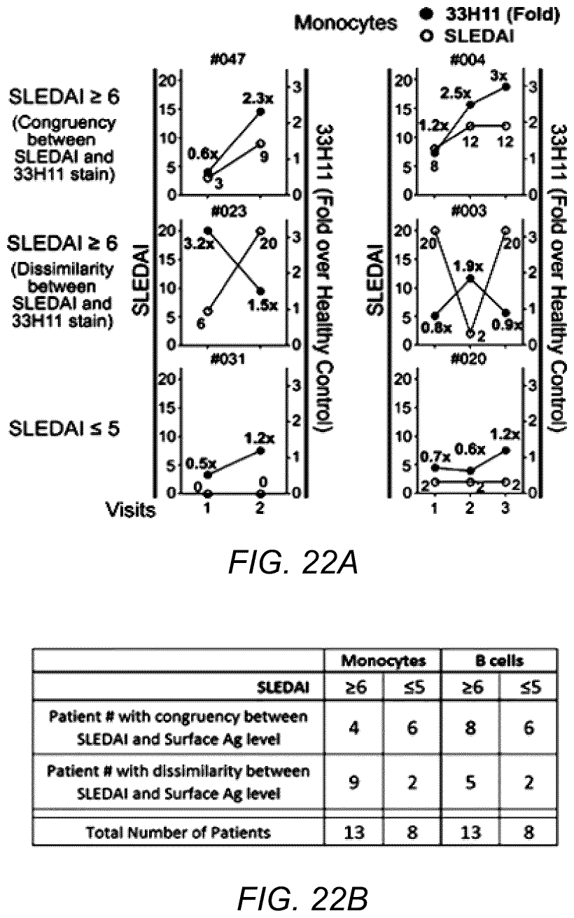

To date, we have analyzed B cells, T cells and monocytes from peripheral blood of 13 active and 8 inactive patients. In active patients, the levels of accumulated nuclear antigens varied between patients; however, 62% of these patients had increased levels of DNA (33H11) on B cells when SLEDAI scores were high (data summarized in FIGS. 22A-22B). We also found that 31% of active patients showed an increase of DNA on monocytes as SLEDAI scores increased (2 examples of patients in FIG. 22B, row 1). Of the remaining patients, the phenotype frequently showed that SLEDAI scores and surface DNA were dissimilar (2 examples of patients in FIG. 22B, row 2). In 70-75% of inactive patients (SLEDAI≤5), the levels of DNA were less than 2-fold over healthy control and SLEDAI scores were low (2 examples of patients in FIG. 22B, row 3). These data suggest that in some patients, the levels of nuclear antigens on B cells show congruence with active disease; however, on monocytes the levels of accumulated nuclear antigens are significantly lower than B cells, and do not parallel SLEDAI changes. This is much like we observed with mouse peripheral blood; however, the sample size is too small to support definitive conclusions.

### Aim 3

Define whether forcibly crosslinking FcγRI/FcγRIIa with FcγRIIb (huSM-PEG [1 ,2]) on monocytes from SLE patients diminishes FcγRI activation and restores lysosomal acidification.

### Rationale and Hypothesis

In mice, defective lysosomal maturation promotes the accumulation of IgG-ICs on hematopoietic cells and is a defect contributing to SLE. Our preliminary data show that MRL/lpr MFs harbor defects in lysosomal acidification coincident with elevated basal levels of pAkt and pS6, a failure to induce SHIP1 phosphorylation, and the inability of pSHIP1 to colocalize with FcγRI (FIGS. 4C, 4D, 19, and 7D). Inhibiting PI3k, mTOR (FIG. 6A), or crosslinking FcγRI and FcγRIIB with SM-PEG[1, 2] (FIGS. 9A left panel, 9C) correct these defects. These findings suggest that in MRL/lpr MFs, FcγRIIb fails to associate with FcγR1, thus preventing SHIP1 activation leading to sustained PIP3 levels. In murine MFs, forcibly crosslinking FcγRI with FcγRIIb activates and recruits pSHIP1 to FcγRI, restoring proper regulation of the PI3k pathway.

The defect is not unique to mice as SLE patients experiencing active disease also show an accumulation of IgG-ICs on peripheral blood hematopoietic cells (FIG. 20). This suggests that recycling of IgG-ICs, heightened activation of the PI3k pathway, mislocalization of pSHIP1, and lysosomal defects might be relevant in human disease. Our goal in this aim is to generate and test two versions of human (hu)SM-PEG[1, 2] that crosslink human FcγRIIb with FcγRI or FcγRIIa. We hypothesize that human monocytes treated with a (hu)SM-PEG[1, 2] will restore lysosomal acidification, reduce activation of PI3k signaling effectors, properly localize SHIP1 at the plasma membrane, and restore lysosomal acidification. This is important in translating our murine findings to human disease, establishing whether FcγRI or FcγRIIa in human is important, and in assessing available antibodies for efficacy in crosslinking. These are all critical factors in deciding whether generating a human version of FcγRI/RIIb-X is worthwhile.

### Aim 4

Test the in vivo efficacy of a murine multivalent, bifunctional biologic designed to co-ligate FcγRI and FcγRIIb.

### Rationale and Hypothesis

This aim follows from our preliminary data showing that crosslinking FcγRI and FcγRIIb using SM-PEG[1, 2] restores lysosomal acidification and diminishes FcγRI activation in MRL/lpr BMMFs. This suggests a defect in colocalizing FcγRIIb/pSHIP1 with aggregates of recycled FcγRI. We hypothesize that crosslinking FcγRI and FcγRIIb will restore lysosomal maturation, and attenuate the onset, or alter the course, of disease in MRL/lpr mice. To test this, we will generate a multivalent, bispecific IgG that forcibly crosslinks FcγRI with FcγRIIb, and that can be used in vivo. This will allow us to assess the disease relevance of impaired lysosomal maturation without the complications of inducing an anti-PEG response. The murine data will also inform whether such a biologic might be efficacious in treating SLE.

### Aim 4a

Generate the tetravalent, bispecific biologic, FcγRI/RIIb-X.

### Experimental Design

A multivalent, bispecific IgG1 (bsAb) will be produced using mutations at the H/L interface that promote proper pairing. This multivalent bsAb will have two Fabs that bind FcγRI (Fab^{FcgRI}), and two that bind FcγRIIb (Fab^{FcgRIIb}). A small number of mutations have been introduced into the Fab region of IgG that enable two Fab moieties to be assembled simultaneously with 95% correct heavy chain (HC)/light chain (LC) pairing. Assembly of the antibodies occurs simply by co-expression. This platform is advantageous over previous iterations of bsAbs because it maintains the C_{H}1 and C_{L} IgG architecture thereby increasing protein stability. As a consequence, these bsAbs show better thermal stability, they maintain good solubility, and they do not aggregate.

The Fab-IgG construct will append two FcγRIIb Fabs at the N-terminus of each heavy chain variable region of FcγRI with a 6xGGGGS linker (SEQ ID NO:10). To accomplish this, we will clone V_{L} and V_{H} gene segments of anti-FcγRI (X54-5/7.1) and anti-FcγRIIb (2.4G2), the antibodies used in SM-PEG[1,2]. These will contain mutations to enforce correct H+L pairing upon co-expression. In addition, the recombinants will have three copies of influenza virus hemagglutinin (HA; YPYDVPDYA; SEQ ID NO:11) on the heavy chain C-terminus. This will allow us to monitor serum half-life. Although HA is immunogenic, it does not elicit anti-HA antibodies in mice. In addition, we will attach a 6xHis-Tag to the C-terminus for protein purification. We will also generate a control construct that does not bind FcγRI or FcγRIIb. To test expression, we will co-transfect the heavy and light chain encoded vectors into suspension HEK293 cells at a 1:1 ratio, and then purify supernatants by Ni⁺⁺-NTA affinity chromatography and gel filtration. We will assess protein size by SDS-PAGE and Western blot, and ensure the construct maintains specificity to FcγRI and FcγRIIb by staining myeloid cells from B6, MRL/pr, B6/FcγRI^{-/-}and B6/FcγRIIb^{-/-}splenocytes. If issues arise in specificity, we will assess heavy:light chain pairing using mass spectrometry. Typically, a given recombinant is designed, expressed, and the binding confirmed within 2-3 months, and ˜100-150 mg of Fab can be produced from 1L of HEK 293 transfectants. Henceforth, we will refer to the multivalent bispecific biologic as “FcγRI/RIIb-X” and the control construct as “control-X.”

### Aim 4b

Characterize the effects of FcγRI/RIIb-X on FcγRI signaling, SHIP1 localization, and lysosomal acidification in vitro.

### Experimental Design

This subaim will test whether the FcγRI/RIIb-X biologic functions comparably to SM-PEG[1, 2] in vitro. BMMFs from B6 and MRLlpr mice will be stimulated with IgG-ICs±SM-PEG[1, 2], or ±FcγRI/RIIb-X (and their respective controls) and the levels of pAKT (S473/T308), and pSHIP1 measured at 15, 60 min and 2 hrs. To assess lysosomal acidification, BMMFs from B6 and MRL/lpr mice will be loaded with Lysosensor™ Yellow/Blue and simultaneously stimulated with IgG-ICs±SM-PEG[1, 2], or ±FcγRI/RIIb-X. pH will be monitored at t=0, 30, and 60 minutes. As a third assessment, we will perform TIRF microscopy to assess whether FcγRI/RIIb-X and SM-PEG[1, 2] recruits SHIP1 to FcγRI. We find that pSHIP1 is colocalized with FcγRI in B6 BMMF stimulated with IgG-ICs. This will be corroborated by confocal microscopy assessing whether pSHIP1 is membrane localized.

### Aim 4c

Assess whether FcγRI/RIIb-X prevents the onset of disease, or restores immune regulation in seropositive MRL/lpr mice.

### Experimental Design

To establish the half-life and treatment frequency of FcγRI/RIIb-X, we will inject B6 (5 mice per dose) and MRL/lpr (5 mice per dose) with 0, 100, 500 ug of FcγRI/RIIb-X. Mice will be bled weekly and HA ELISAs performed to assess the serum levels of IgG1 for up to 1 month. To establish optimal dosing, cohorts of 4 week old B6 (5 mice per dose) and MRL/lpr mice (5 mice per dose) will be injected with 0, 100, 250, or 500 ug of FcγRI/RIIb-X for 12 weeks at a frequency determined in the pilot study of half-life. We will pre-bleed mice and after the injection protocol begins, bleed monthly to assess anti-nucleosome, pAKT (S473/T308) (from peripheral blood monocytes) and urine protein (URISTIX).

The foregoing is illustrative of the present invention, and is not to be construed as limiting thereof. The invention is defined by the following claims, with equivalents of the claims to be included therein.

That which is claimed is:

1. A method for treating an autoimmune disorder in a subject, comprising administering to the subject an effective amount of a molecule comprising:
at least one of a first immunoglobulin fragment antigen binding region (Fab1) and at least one of a second immunoglobulin fragment antigen binding region (Fab2), wherein Fab1 is specific for FcγRI or FcγRIIa, and wherein Fab2 is specific for FcγRIIb.

2. The method of claim 1, wherein Fab1 is specific for FcγRI.

3. The method of claim 1, wherein the molecule comprises a bispecific antibody.

4. The method of claim 1, wherein the molecule comprises at least two of a second Fab2.

5. The method of claim 4, comprising a first Fab2, a second Fab2, and a first Fab1, wherein the Fab1 is linked to the first Fab2, and wherein the first Fab2 is further linked to the second Fab2.

6. The method of claim 4, further comprising an immunoglobulin Fc region.

7. The method of claim 6, comprising a first Fab1, a first Fab2, and a second Fab2, wherein the first Fab1 is linked to the Fc region, wherein the first Fab2 is linked to the Fc region, and wherein the second Fab2 is linked to the first Fab1.

8. The method of claim 6, comprising a first Fab1, a second Fab1, a first Fab2, and a second Fab2, wherein the first Fab1 and the second Fab1 are linked to the Fc region, and wherein the first Fab2 is linked to the first Fab1 and the second Fab2is linked to the second Fab1.

9. The method of claim 1, wherein the autoimmune disorder is systemic lupus erythematosus (SLE).

10. The method of claim 1, wherein the autoimmune disorder is diabetes.

11. The method of claim 1, wherein the autoimmune disorder is arthritis.
